(19) United States
(12) Patent Application Publication
(10) Pub. No.: US 2012/0251999 A1
(43) Pub. Date: Oct. 4, 2012

(54) VITRIFICATION SYSTEMS AND METHODS

(75) Inventors: Utkan DEMIRCI, Cambridge, MA (US); Emre KAYAALP, Tenafly, NJ (US); Sangjun MOON, Allston, MA (US); Young Seok SONG, Cambridge, MA (US); Xiaohui ZHANG, Westmont, IL (US)

(73) Assignee: THE BRIGHAM AND WOMEN'S HOSPITAL, INC., Boston, MA (US)

(21) Appl. No.: 13/364,143

(22) Filed: Feb. 1, 2012

Related U.S. Application Data
(60) Provisional application No. 61/438,336, filed on Feb. 1, 2011.

(51) Int. Cl. G01N 1/42; C12N 5/078; C12N 5/074; C12N 5/077; C12N 5/0789; C12M 1/04; C12N 5/076; C12N 5/0775; C12N 5/071; C12M 1/00; C12M 1/42; C12N 5/0735; C12N 5/075

(52) U.S. Cl. 435/2; 436/176; 435/374; 435/307.1; 422/501

(57) ABSTRACT

The embodiments of the invention described herein relate to systems and methods for the vitrification of biological samples. Vitrification is achieved by generating nanodroplets of a solution comprising the biological sample with a means that can be automated and adapted to high-throughput applications.

### CROSS REFERENCE TO RELATED APPLICATIONS

This claims the benefit under 35 U.S.C. 119(e) of U.S. Provisional Patent Application Ser. No. 61/438,336 filed Feb. 1, 2011, the contents of each of which are herein incorporated by reference in their entirety.

### GOVERNMENT SUPPORT

This invention was made in part with U.S. Government support from grants RO1A1081534 and R12 A1087107 from the National Institutes of Health. The U.S. Government has certain rights in this invention.

### FIELD OF THE INVENTION

The systems and methods of the invention as described herein relate to the cryopreservation of biological samples.

### BACKGROUND

Long-term preservation of cells and tissues through cryopreservation has broad impacts in multiple fields including tissue engineering, fertility and reproductive medicine, regenerative medicine, stem cells, blood banking, animal strain preservation, clinical sample storage, transplantation medicine and in vitro drug testing. Traditional cryopreservation techniques (e.g. high glycerol/slow freezing; low glycerol/rapid freezing; and slow freezing-rapid thawing) result in significant levels of cellular injury and/or death as a result of cell shrinkage (Zdeoppe, Acta Physiologica Scandinavia 1968 73:341), toxicity due to the increasing concentrations of solutes (Pegg and Diaper. Cyrobiology 1991 28:18-35; Pegg and Diaper. Biophysical Journal 1988 54:471-488) during slow freezing, and ice formation (Mazur et al. Exp Cell Res 1972 71:345-355; Gratwohl. Eur J Haematol 2010 84:95). The process of vitrification, a rapid cooling that results in a glass structure at the molecular level, eliminates ice crystal formation and results in improvement in post-thaw cell viability and function compared to the traditional freezing methods. Vitrification methods have applications including reproductive cells, stem cells, blood and tissue engineered constructs (TECs). Vitrification using minimum sample volume enables increased cooling and warming rates and the utilization of lower CPA concentrations, thus reducing toxicity and osmotic shock to cells/tissues. Minimum volume vitrification systems consist of: carrier-based and carrier-free (droplet-based vitrification) systems. However, existing minimum volume vitrification methods are operationally demanding, suffer from low throughput, and require a high level of technical skill.

### SUMMARY OF THE INVENTION

Described herein are methods and systems relating to vitrification of biological samples. Aspects of the invention described herein are based in part upon the inventors' discovery of how to reproducibly generate cell-encapsulating nanodroplets. Aspects of the invention described herein are based in part upon the inventors' discovery of how to reproducibly generate cell-encapsulating nanodroplets of less than 500 nL. Aspects of the invention described herein are based in part upon the inventors' discovery of how to reproducibly generate cell-encapsulating nanodroplets using systems and methods not requiring manual operation and which are amenable to high throughput applications. The methods and systems described herein can reduce the level of technical skill required to perform the vitrification process, result in consistently-sized nanodroplets, and are amenable to automation and high through-put. Furthermore, aspects of the invention described herein relate to carrier-free vitrification, resulting in desirable higher cooling and warming rates.

In one aspect, the embodiments of the invention described herein relate to a method of vitrifying a biological sample comprising; a) generating nanodroplets of a solution comprising the biological sample; and b) contacting the nanodroplets with a cooling agent.

In some embodiments, the biological sample is selected from the group consisting of: cells; biological fluids; biopsy samples; diagnostic samples; blood; urine; and protein. In some embodiments, the cells are selected from the group consisting of: gametes; sperm; eggs; embryos; zygotes; chondrocytes; red blood cells; blood cells, hepatic cells, fibroblasts, stem cells; cord blood cells; adult stem cells, induced pluripotent stem cells, autologous cells; autologous stem cells; bone marrow cells; hematopoietic cells; embryonic stem cells; and hematopoietic stem cells.

In some embodiments, the nanodroplets have a volume of less than 500 nL. In some embodiments, the nanodroplets have a volume of less than 100 nL. In some embodiments, the nanodroplets have a volume of less than 10 nL.

In some embodiments, the solution comprising the biological sample further comprises at least one cryoprotective agent. In some embodiments, the cryoprotective agent is selected from the group consisting of dimethylsulphoxide (DMSO), 1,2-propanediol (PROH), ethylene glycol (EG), sucrose, trehalose; mannitol; ectoin; methylcellulose; polyethylene glycol (PEG); and naturally occurring cyroprotectants.

In some embodiments, the cryoprotective agent is present at a concentration of less than 6 M. In some embodiments, the cryoprotective agent is present at a concentration of less than 3 M. In some embodiments, the cryoprotective agent is present at a concentration of less than 2 M.

In some embodiments, the biological agent further comprises a hydrogel.

In some embodiments, the nanodroplet is generated by causing the solution comprising the biological sample to flow through a nozzle of a reservoir. In some embodiments, the solution comprising the biological sample is caused to flow through the nozzle of the reservoir via a means selected from the group consisting of: a plunger; a solenoid-controlled plunger; co-flow of a gas; an inkjet, and spraying.

In some embodiments, the means of generating the nanodroplets is an acoustic generator.

In some embodiments, the means of generating nanodroplets is automated.

In some embodiments, the nanodroplets are contacted with a cooling agent by allowing the nanodroplets to fall from the nozzle into or onto a cooling agent. In some embodiments, the nanodroplets are contacted with a cooling agent by allowing the nanodroplets to fall from the outflow opening onto a collection membrane and then contacting the collection membrane with a cooling agent. In some embodiments, the cooling agent is selected from the group consisting of: liquid nitrogen, nitrogen vapor, liquid helium, and helium vapor.

In some embodiments, the method further comprises storing the vitrified biological sample at a temperature lower than −130° C.

In some embodiments, the method further comprises generating nanodroplets of a solution in a high throughput system. In some embodiments, the high throughput system comprises a reservoir with multiple outflow openings. In some embodiments, the high throughput system comprises multiple reservoirs.

In some embodiments, the method further comprises the step of causing the vitrified biological sample to warm rapidly.

In one aspect, the embodiments of the invention as described herein relate to a system for vitrifying a biological sample comprising; a) a reservoir containing the biological sample; b) a means of forming the biological sample into nanodroplets and directing the nanodroplets to flow or fall towards a catchment; and c) a catchment for collecting the nanodroplets.

In some embodiments, the means of forming the biological sample into nanodroplets comprises causing the solution comprising a biological sample to flow through a nozzle. In some embodiments, the means of causing a biological sample to flow through the nozzle connected to the reservoir is selected from a group consisting of: a plunger; a solenoid-controlled plunger; a gas co-flow muzzle; and spraying. In some embodiments, the terminus of the nozzle is less than 200 μm in diameter.

In some embodiments, the means of forming the biological sample into nanodroplets is an acoustic generator.

In some embodiments, the means of forming the biological sample into nanodroplets is automated.

In some embodiments, the catchment comprises a cooling agent. In some embodiments, the cooling agent is selected from the group consisting of: liquid nitrogen, nitrogen vapor, liquid helium, and helium vapor.

In some embodiments, the catchment comprises a collection membrane.

In some embodiments, the nanodroplets have a volume of less than 500 nL. In some embodiments, the nanodroplets have a volume of less than 100 nL. In some embodiments, the nanodroplets have a volume of less than 10 nL.

In some embodiments, the solution comprising the biological sample further comprises at least one cryoprotective agent. In some embodiments, the cryoprotective agent is selected from the group consisting of: dimethylsulphoxide (DMSO), 1,2-propanediol (PROH), ethylene glycol (EG), sucrose, trehalose; mannitol; ectoin; methylcellulose; polyethylene glycol (PEG); and naturally occurring cyroprotectants.

In some embodiments, the biological agent further comprises a hydrogel.

In some embodiments, the cryoprotective agent is present at a concentration of less than 6 M. In some embodiments, the cryoprotective agent is present at a concentration of less than 3 M. In some embodiments, the cryoprotective agent is present at a concentration of less than 2 M.

In some embodiments, the system is a high throughput system. In some embodiments, the high throughput system comprises a reservoir with multiple nozzles. In some embodiments, the high throughput system comprises multiple reservoirs.

### BRIEF DESCRIPTION OF THE DRAWINGS

FIGS. 1A-1B depict the droplet generating system for the blood cryopreservation process. FIG. 1A depicts a schematic description for blood droplet ejection on collection film and images of droplets. Scale bar is 500 μm. FIG. 1B depicts a graph of size distribution of ejected droplets. Average droplet size and standard deviation are shown as a function of droplet collecting distance, sheath gas flow rate, and blood flow rate. Error bars are standard deviations. FIGS. 1C-1E depict images of human RBCs. FIG. 1C depicts RBCs before freeze/thaw, and FIGS. 1D-1E depict RBCs following freezing and thawing steps. The RBC morphology was conserved.

FIGS. 2A-2C depict an overall schematic of the blood cryopreservation setup and process steps. FIG. 2A depicts the CPA loading process for whole blood, FIG. 2B depicts the vitrification process and FIG. 2C depicts a picture of multi-ejector setup, ejector length (Lext) and needle tip (Dneedle) was 3.0 mm outer diameter and 210 μm inner diameter (27 gauge needle). Scale bar is 1 mm.

FIGS. 3A-3D depict droplet size measurements. FIG. 3A depicts images of ejected droplets on collection film. Outline tracking, measuring size, and counting of droplets using Image J software. Droplets were collected on paper with three different distances and sheath flow rates at a fixed blood flow rate, 200 μl/min. Droplet diameters were calculated using the area of each closed outline. FIGS. 3B-3D depict graphs of droplet size distribution shown as a function of droplet collecting distance, gas flow rate, and blood flow rate. FIG. 3B depicts data obtained with a distance of 60 mm; FIG. 3C depicts data obtained with a distance of 75 mm, and FIG. 3D depicts data obtained with a distance of 90 mm.

FIGS. 4A-4D depict percent hemolysis values for ejection and freezing at five different conditions. FIG. 4A depicts Cripps method values and FIG. 4B depicts Harboe method values. P-values were tested at two different distances (60 and 90 mm) and gas flow rates (3.2 and 4.8 l/min), Table 6. Percent hemolysis values of parallel ejection systems were shown for (FIG. 4C) 4 parallel ejectors and (FIG. 4D) 25 parallel ejectors system. Both 4 and 25 ejector systems were operated at 3.2 l/min and 6 mm.

FIGS. 5A-5D depict absorbance values and percent hemolysis for different steps during cryopreservation process. Percent hemolysis was measured for each process step following Eqn. S1 as described in the Percent Hemolysis Analysis section in the manuscript. Each dotted box represents how each process affects hemolysis, i.e. (FIG. 5A) CPA1 loading effect (ABSCPA1), (FIG. 5B) CPA2 loading effect (ABSCPA2), (FIG. 5C) ejection effect (ABSejection), and (FIG. 5D) freezing and thawing (ABSfreeze) effect.

FIGS. 6A-6D depict the ejector-based droplet generation setup and the overview of stepwise experimental procedures for evaluating each step for oocyte survival described in Example 2. FIG. 6A depicts the experimental setup for oocyte encapsulating droplet ejection into the droplet receiving container. FIG. 6B depicts that CPA was loaded to and unloaded from oocytes sequentially. FIG. 6C depicts that oocytes were ejected into the thawing medium (W1) after CPA loading followed by CPA unloading. FIG. 6D depicts that after CPA loading, oocytes encapsulated in CPA droplets were ejected into LN on an Al foil (collection sieve), thawed in thawing medium (W1) followed by CPA unloading. Oocytes retrieved from each procedure (FIGS. 6B-6D) were transferred to the medium and cultured at 37° C. before survival analysis. Al: Aluminum; CPA: Cryoprotectant agent; KSOM: Potassium simplex optimized medium; LN: Liquid nitrogen; W1: Warming solution 1.

FIGS. 7A-7B depict droplet sizes obtained at various nitrogen gas flow rates and the droplet size distribution at 0.8 sLPM. FIG. 7A depicts that average droplet size was observed to decrease as nitrogen gas flow rate increased up to 0.9 SPLM. For nitrogen gas flow rate values higher than 0.9 SLPM, the droplet size mean values were similar. Error bars indicate standard deviations. FIG. 7B depicts that droplet size distribution ejected at 0.8 SLPM displayed a normal distribution with left hand skew. SLPM: Standard liter per min.

FIGS. 8A-8H depict morphological observations of oocytes at each procedure step. Surviving oocytes from each procedure showed no difference compared with the controls in morphology. FIGS. 8A-8B depict fresh oocytes (control); FIGS. 8C-8D depict oocytes recovered after CPA loading and unloading; FIGS. 8E-8F depict oocytes after ejection with CPA; and FIGS. 8G-8H depict oocytes after vitrification and thawing steps. Images were taken 30 min (FIGS. 8A, 8C, 8E, and 8G) and 24 h (FIGS. 8B, 8D, 8F, and 8H) after each procedure. CPA: Cryoprotectant agent.

FIGS. 9A-9F depict embryo development of parthenogenetically activated murine oocytes using 50 mM SrCl₂. Embryo cleavage was evaluated daily. FIG. 9A depicts oocytes before activation on day 0; FIG. 9B depicts two-cell embryos on day 1; FIG. 9C depicts four-cell to six-cell embryos on day 2; FIG. 9D depicts eight-cell to 12-cell embryos on day 3; FIG. 9E depicts morula on day 4 and blastocysts (FIG. 9F) on day 5. The images were taken with a 10× objective and the insets were taken with a 20× objective. White arrows indicate abnormal embryos. Black arrows indicate blastocysts.

FIGS. 10A-10D depict an overview of Example 3. FIG. 10A depicts the microfluidic device which exposes cells to CPAs gradually. FIG. 10B depicts a schematic of how cells exposed to CPAs are placed in droplets, and then ejected into liquid nitrogen. FIGS. 10C-10D depict a high throughput array of 25 ejectors that can vitrify a unit of blood about 20 minutes.

FIGS. 11A-11C depict the characterization of the correlation between droplet size and CPA concentration. FIG. 11A depicts a graph demonstrating that small droplets vitrify at low CPA concentration. The blue circles indicate the largest droplets that vitrified. The insert (lower right corner) shows vitrified droplets whereas those in the top-left insert are nonvitrified. FIG. 11B depicts droplets of various size ejected into liquid nitrogen. The vitrified droplets appear clear and translucent, whereas the frozen non-vitrified droplets appear cloudy and dark. A nonvitrified droplet is marked with an arrow. FIG. 11C depicts a zoom-in image of the droplets (60×), revealing droplet structure.

FIG. 12 depicts a schematic of a CPA loading cell cryopreservation microchip.

FIG. 13 depicts a schematic of freezing/thawing.

FIG. 14 depicts a schematic of a CPA unloading (using PBS solution) cell cryopreservation microchip.

FIG. 15 depicts cryopreservation steps using novel droplet technologies to push the vitrification limits for the droplet size and CPA concentrations below the current levels available with existing clinical technologies.

FIGS. 16A-16B depict steps of vitrification and thawing process using (FIG. 16A) stepwise or (FIG. 16B) microfluidic CPA loading/unloading.

FIGS. 17A-17B depict specific embodiments of the system described herein. FIG. 17B depicts a close-up view of a gas coflow muzzle and nozzle.

FIG. 18 depicts a simplified diagram of one embodiment of the system described herein.

FIG. 19 depicts one embodiment of the system described herein.

### DETAILED DESCRIPTION OF THE INVENTION

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8).

Unless otherwise stated, the present invention was performed using standard procedures, as described, for example in U.S. Pat. Nos. 4,965,343, and 5,849,954; Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Cell Biology (CPCB) (Juan S. Bonifacino et. al. ed., John Wiley and Sons, Inc.); Culture of Animal Cells: A Manual of Basic Technique by R. Ian Freshney, Publisher: Wiley-Liss; 5th edition (2005); and Animal Cell Culture Methods (Methods in Cell Biology, Vol. 57, Jennie P. Mather and David Barnes editors, Academic Press, 1st edition, 1998) which are all incorporated by reference herein in their entireties.

The terms “decrease,” “reduce,” “reduced”, and “reduction” are all used herein generally to mean a decrease by a statistically significant amount relative to a reference. However, for avoidance of doubt, “reduce,” “reduction”, or “decrease” typically means a decrease by at least 10% as compared to the absence of a given treatment and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the absence of a given treatment, or any decrease between 10-99% as compared to the absence of a given treatment.

The terms “increased”, “increase”, or “enhance” are all used herein to generally mean an increase by a statically significant amount; for the avoidance of any doubt, the terms “increased”, “increase”, or “enhance” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “port” refers to a portion of a system described herein which provides a means for fluid and/or cells to enter and/or exit that portion of the system. The port can be of a size and shape to accept and/or secure a connection with tubes, connections, or adaptors of a microfluidics system and allow passage of fluid and/or cells when attached to a microfluidics system.

The term “encapsulate” as used herein refers to surrounding a portion of a biological sample (e.g. a cell, a population of cells, an embryo, or one or more polypeptides) with a solution such that the portion of the biological sample is contained within the solution. In some embodiments, the volume of the solution encapsulating the portion of the biological sample is less than 500 nL. In some embodiments, the solution encapsulating the portion of the biological sample is a nanodroplet.

The term “isolated” as used herein in reference to cells refers to a cell that is mechanically separated from another group of cells with which they are normally associated in vivo. Examples of a group of cells are a developing cell mass, a cell culture, a cell line, and an animal. These examples are not meant to be limiting. Methods for isolating one or more cells from another group of cells are well known in the art. See, e.g., Culture of Animal Cells: a manual of basic techniques (3rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; Animal Cells: culture and media, 1994, D. C. Darling, S. J. Morgan, John Wiley and Sons, Ltd.

The terms “subject” and “individual” are used interchangeably herein, and refer to an organism, for example, a human, from which a sample is obtained. A subject can be any organism for which it is desired to determine the presence of a nucleic acid in the organism or one or more cells comprising or contained within that organism.

As used herein, a subject can mean an organism, e.g. a bacterium, a parasite, a plant, or an animal. As used herein, a “subject” can mean a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Individual or subject includes any subset of the foregoing, e.g., all of the above. In certain embodiments, the subject is a mammal, e.g., a primate, e.g., a human.

Other than in the operating examples, or where otherwise indicated, all numbers expressing quantities of ingredients or reaction conditions used herein should be understood as modified in all instances by the term “about.” The term “about” when used in connection with percentages can mean±1%.

The term “statistically significant” or “significantly” refers to statistical significance and generally means a two standard deviation (2SD) difference, above or below a reference value. Additional definitions are provided in the text of individual sections below.

Cryopreservation involves the storage of biological samples at temperatures at which biological activity effectively ceases. This allows storage, of biological samples with minimal degradation of the sample and/or long-term storage of biological samples. However, traditional cryopreservation techniques are hampered by the formation of ice crystals and the increase in ionic strength of unfrozen concentrated solutions, both of which cause damage to the biological sample.

Vitrification offers improved outcomes by preventing the formation of ice crystals and the increase in ionic strength of unfrozen concentrated solutions (See Zhang et al. Nanomedicine 2011 6:1115-1129). Vitrification refers to conversion of liquid phase directly into a glass-like solid. Vitrification techniques require higher cryopreservation agent (CPA) concentrations (e.g., 6˜8 M) and higher cooling rates (e.g., −1500° C./min) compared to slow freezing methods. Rapid cooling rates can be achieved by immersing cells/tissues directly into liquid nitrogen (−196° C.) or liquid nitrogen vapor (−160° C.). High CPA levels used in vitrification lower the freezing point and increase the medium viscosity. However, it is known that these high CPA levels cause osmotic shock and toxicity to cells resulting in alterations in cytoskeleton, spindle disassembly and chromosome dispersal.

Attempts to reduce the volume of samples being vitrified have been explored as a means to reduce the adverse effects of high CPA concentrations. However, existing methods are operationally demanding and require a high level of technical skill, reducing their clinical desirability and preventing their application to high-throughput cryopreservation needs (e.g. blood storage). Further, the repeatability and consistency of existing methods is suboptimal due to the manual nature of the protocols.

In some embodiments, the systems and methods described herein relate to the generation of nanodroplets. As used herein, a “nanodroplet” is an aliquot, portion, or volume of a solution having a volume of less than 750 nL, e.g. a volume of 700 nL, 500 nL, 300 nL, 200 nL, 100 nL, 75 nL, 50 nL, 25 nL, 10 nL, 1 nL or less. The size of the nanodroplet generated can be controlled by the droplet collection distance 32; the size of the droplet formation port 22; and the rate of the flow of solution comprising a biological sample through the nozzle 20. A nanodroplet can be spherical, ovoid, oval, or irregular in shape. In some embodiments, at the time of contacting a cooling agent, the nanodroplet is spherical or approximately spherical in shape. The nanodroplet volume can be determined, for example by determining the radius or diameter of a spherical nanodroplet via microscopic examination.

In some embodiments a nanodroplet is from 1 nL to 750 nL in volume. In some embodiments a nanodroplet is from 1 nL to 500 nL in volume. In some embodiments a nanodroplet is from 10 nL to 400 nL in volume. In some embodiments a nanodroplet is from 20 nL to 300 nL in volume. In some embodiments a nanodroplet is from 30 nL to 250 nL in volume. In some embodiments a nanodroplet is from 40 nL to 200 nL in volume. In some embodiments a nanodroplet is from 50 nL to 100 nL in volume. In some embodiments a nanodroplet is from 10 nL to 100 nL in volume. In some embodiments a nanodroplet is from 100 nL to 200 nL in volume. In some embodiments, a nanodroplet has a volume of less than 500 nL. In some embodiments, a nanodroplet has a volume of less than 450 nL. In some embodiments, a nanodroplet has a volume of less than 400 nL. In some embodiments, a nanodroplet has a volume of less than 350 nL. In some embodiments, a nanodroplet has a volume of less than 300 nL. In some embodiments, a nanodroplet has a volume of less than 250 nL. In some embodiments, a nanodroplet has a volume of less than 200 nL. In some embodiments, a nanodroplet has a volume of less than 150 nL. In some embodiments, a nanodroplet has a volume of less than 100 nL. In some embodiments, a nanodroplet has a volume of less than 75 nL. In some embodiments, a nanodroplet has a volume of less than 50 nL. In some embodiments, a nanodroplet has a volume of less than 25 nL. In some embodiments, a nanodroplet has a volume of less than 10 nL. In some embodiments, a nanodroplet has a volume of less than 10 nL. In some embodiments, a nanodroplet has a volume of less than 5 nL. In some embodiments, a nanodroplet has a volume of less than 2 nL. In some embodiments, a nanodroplet has a volume of less than 1 nL.

In some embodiments, the size of a nanodroplet can be expressed as the dimensionless radius (r*). The value r* is equal to r/R, where R is the radius of the nanodroplet and r is the coordinate in the radius direction. In some embodiments, the dimensionless radius (r*) of a nanodroplet is 0.1 or lower, e.g. the dimensionless radii are 0.1, or 0.09, or 0.08, or 0.05, or 0.03, or 0.01 or lower.

In some embodiments, the volumes and/or diameters of a group of nanodroplets generated under identical conditions according to the systems and methods described herein should vary by less than 60% of the average for the group, e.g. by less than 50% of the average for the group, by less than 40% of the average for the group or less.

The preferred size of a nanodroplet can vary depending upon the biological sample that is to be vitrified. In some embodiments, a nanodroplet can encapsulate a single entity of the biological sample, e.g. a single cell, a single piece of tissue, a single embryo, or a single polypeptide. In some embodiments, a nanodroplet can encapsulate multiple entities of the biological sample, e.g. multiple cells, multiple pieces of tissue, multiple embryos, multiple proteins. The number of entities encapsulated by a nanodroplet is determined by droplet size and the concentration of the entities in the biological sample. In some embodiments, the nanodroplet can be of a size sufficient to encapsulate 1 or more cells, e.g. 1 cell, 2 cells, 3 cells, 5 cells, 7 cells, 10 cells or more cells can be encapsulated within a single nanodroplet.

By way of non-limiting example, the nanodroplet should have a volume of at least 1.5 times the volume of the portion of the biological sample being encapsulated, e.g. at least 1.5 times, at least 3.5 times, at least 3 times, at least 4 times, at least 5 times or greater than the volume of the portion of the biological sample being encapsulated. By way of non-limiting example, under the conditions described in Example 2, for mouse oocytes with an average volume of 0.4 nL, a nanodroplet volume of 1.4 nL was found to be optimal, i.e. the nanodroplet had a volume 3.5 of that of the portion of the biological sample being encapsulated.

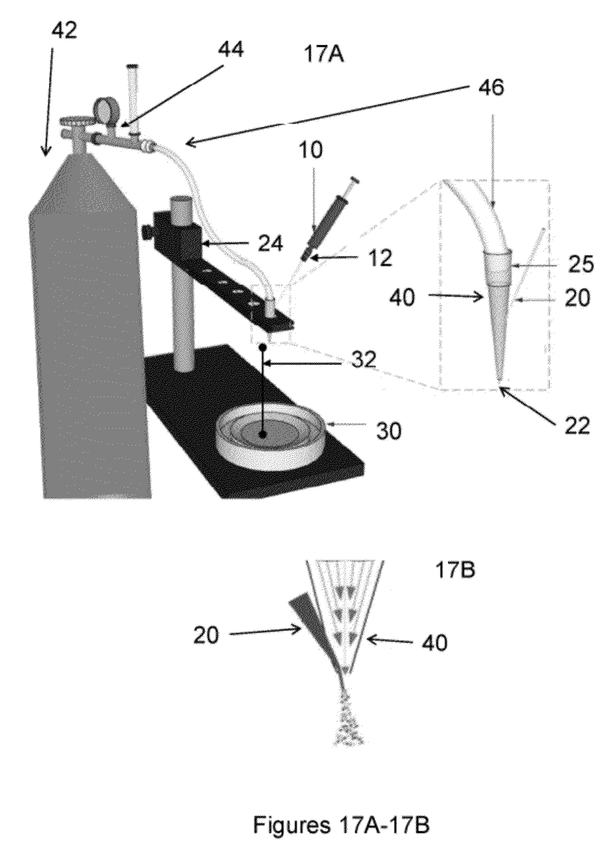

FIG. 17A depicts one embodiment of a system for vitrifying a biological sample as described herein. In some embodiments, the system illustrated in FIG. 17A can be used to effect the methods described herein. It should be noted that the embodiments shown in the Figures are exemplary and not meant to be limiting.

To achieve the set up illustrated in FIG. 17A, a gas reservoir 42 is provided with a gas flow regulator 44, gas tubing 46 and gas coflow muzzle 40. Reservoir 10 having port or opening 12 is coupled to gas coflow muzzle 40 by way of nozzle 20. Gas coflow muzzle 40 and/or nozzle 20 are coupled to platform 24 in a desired position or orientation, such that droplet formation port 22 is positioned above a catchment 30. It is contemplated that gas reservoir 42, gas flow regulator 44, gas tubing 46, gas coflow muzzle 40, reservoir 10, port or opening 12, nozzle 20 and/or catchment 40 can be multiuse or disposable.

A nozzle 20 can be of plastic, metal, glass or any other suitable material in the approximate shape of a hollow funnel or cylinder, where the distal end of the nozzle 20 furthest from the reservoir 10 has an opening of less than 300 um, e.g. less than 275 um, less than 250 um, less than 200 um, less than 150 um, less than 100 um or smaller. In accordance with some embodiments of the invention, the nozzle can be formed by molding, machining, micro-machining, etching, and/or casting into forms that provide the basic elements of the nozzle. In some embodiments, the nozzle is comprised of portions and/or layers. In some embodiments, adhesives can be used to adhere the portions and/or layers together to form the nozzle. Any suitable adhesive may be used to adhere the portions and/or layers together.

In some embodiments, a nozzle 20 can be a needle tip. In some embodiments, a nozzle 20 can be a 27 gauge stainless needle tip. In some embodiments, a nozzle 20 can be a STRIPPER® tip. A nozzle 20 can be connected to a reservoir 10 directly or indirectly (e.g. via tubing). Tubing can be plastic tubing, e.g. polyethylene tubing, microfluidics system tubing, combinations thereof, and the like. A nozzle 20 is referred to interchangeably herein as an “ejector.” A system as described herein comprising at least a reservoir 10 and a nozzle 20 is referred to interchangeably herein as an “ejector-based droplet system.”

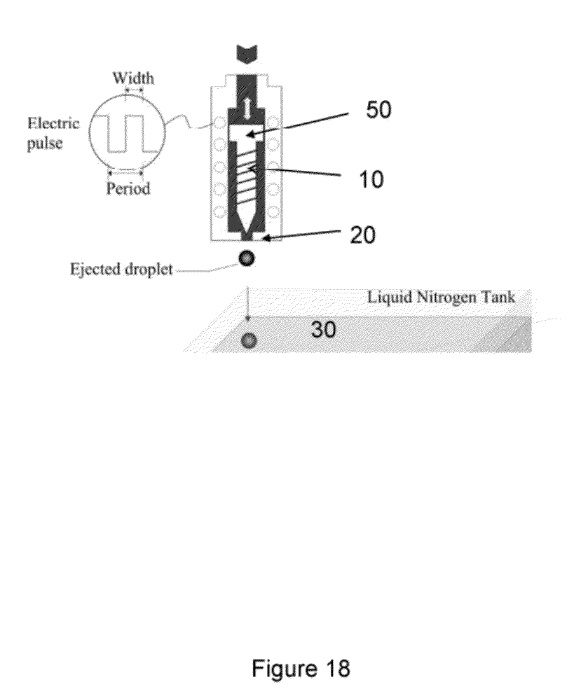

In some embodiments, a single structure can comprise both a nozzle 20 and reservoir 10. For example, in the embodiment depicted in FIG. 18, a syringe comprises both the reservoir 10 and the nozzle 20.

A reservoir 10 can be made of a flexible or a non-flexible material according to the design and application requirements. The reservoir 10 can be made of plastic, metal, glass, PDMS, polyurethane, silicon, polysulfane, ceramics, polymers, hard plastic and the like, as well as a combination of these materials. The reservoir 10 can be made in any shape having an interior space capable of accepting a biological sample and/or solution comprising a biological sample.

The reservoir 10 has a port or opening 12 through which the biological sample and/or solution comprising the biological sample can enter the reservoir 10. Port or opening 12 can be directly or indirectly (e.g. with tubing) connected to the nozzle 20. In some embodiments, the port 12 for entry of the biological sample and the port 12 for connecting to the nozzle, can be the same port, e.g. such as in the case of a syringe. In some embodiments, the reservoir 10 can be a syringe. In some embodiments, the reservoir 10 can be a pipette. In some embodiments, the reservoir 10 can be a STRIPPER® pipette. In some embodiments, the reservoir 10 can be a blood bag.

In accordance with some embodiments of the invention, the reservoir can be formed by molding biocompatible plastic materials in layers that can be bonded together using adhesives to form the chambers and the passage ways. Other materials, such as glass and biocompatible metals (steel, titanium, alloys) can also be used to fabricate the layers that can be used to form reservoir. Glass and metallic materials can be machined, micro-machined, etched, and cast into forms that provide the basic elements of the reservoir. Similarly, adhesives can be used to adhere the layers together to form the reservoir.

A platform 24 provides a means of orienting and/or positioning the nozzle(s) 20, gas coflow muzzle 40, or other means of generating nanodroplets so that the nanodroplets fall a desired droplet collection distance 32 into a catchment 30. A platform 24 can further comprise a droplet generation means (e.g. a gas coflow muzzle 40). In this embodiment, platform 24 can alternatively or additionally provide a means of orienting and/or positioning the gas coflow muzzle 40. For example, platform 24 can orient and position gas coflow muzzle 40, the positioning of which in itself orients and positions nozzle 20.

A platform 24 can position or orient a nozzle 20 and/or a gas coflow muzzle 40 by having slots, sockets, clamps, screws, a matrix, a substrate, adhesives or any other means of holding the nozzle 20 in position. Exemplary platforms are illustrated in the Figures herein. A platform 24 can be made of plastic, glass, metal, polymer and the like or combinations thereof. In some embodiments, the platform 24 is made of poly(methyl methacrylate). In accordance with some embodiments of the invention, the platform can be formed by molding, machining, micro-machining, etching, and/or casting into forms that provide the basic elements of the platform. In some embodiments, the platform is comprised of portions and/or layers. In some embodiments, adhesives can be used to adhere the portions and/or layers together to form the platform. Any suitable adhesive may be used to adhere the portions and/or layers together to form the platform.

In use, gas reservoir 42 is opened to allow for the flow of gas. The gas contained within gas reservoir 42 can be compressed, and can contain any gas which is not toxic to a biological sample. For example, the gas can be nitrogen gas, oxygen gas, helium gas or mixtures thereof. The flow of gas can be regulated by a gas flow regulator 44, which acts as a pressure regulator that adjusts the flow of gas from gas reservoir 42 to an appropriate pressure, the selection of which is described further herein. The gas flow regulator 44 can be analog or digital, and the appropriate pressure can be selected and regulated manually or automatically. The gas flows through gas tubing 46 to gas coflow muzzle 40.

A biological sample and/or a solution comprising the biological sample is contained in reservoir 10. In one embodiment in which reservoir 10 is a syringe, nozzle 20 is placed in the solution, and a plunger is pulled outward. The solution moves through nozzle 20 and into reservoir 10 via port or opening 12.

In this embodiment, nozzle 20 then pierces gas coflow muzzle 40. The biological sample and/or the solution comprising the biological sample is ejected into gas coflow muzzle 40. In the case of a syringe, for example, the solution may be ejected by depressing the plunger on a syringe. Although described with respect to depressing a plunger, however, the flow of the biological sample and/or solution comprising the biological sample can be caused and/or controlled by any suitable means, e.g. via a syringe pump (e.g. Cat # SPLG212; World Precision Instrument; Sarasota Fla.). In some embodiments, the flow of the solution can be automated. In some embodiments, the flow of the solution can be controlled manually. In some embodiments, the flow of the solution can be controlled by a solenoid. In embodiments in which a syringe pump is used, the flow of the solution can be automated.

Nanodroplets are then released from a droplet formation port 22, and form at a point coincident with or outside of the physical terminus of the nozzle 20. The nozzle 20, and optionally the reservoir 10, are kept in the desired orientation and position by a platform 24. The nozzle is positioned and/or oriented by the platform 24 such that the flow of nanodroplets created will flow, fall, spray, or otherwise traverse the distance (i.e. droplet collection distance 32) to a catchment 30.

The droplet collection distance 32 can be varied in order to vary the size of the nanodroplet generated according to some embodiments. In some embodiments, the droplet collection distance 32 can be from 10 mm to 10 cm. In some embodiments, the droplet collection distance 32 can be from 60 mm to 90 mm. In some embodiments, the droplet collection distance 32 can be about 60 mm. In some embodiments, the droplet collection distance 32 can be about 75 mm. In some embodiments, the droplet collection distance can be about 90 mm. In some embodiments, the droplet collection distance 32 is large enough such that if a cooling agent is present in the catchment 30, vapor from the cooling agent does not visibly contact the droplet formation port 22. In some embodiments, the droplet collection distance 32 is large enough such that if a cooling agent is present in the catchment 30, the temperature at the droplet formation port 22 is the same when cooling agent is present in the catchment 30 as when the cooling agent is not present in the catchment 30.

A catchment 30 can comprise a surface, depression, container, matrix, or solution in which the nanodroplets are collected. A catchment 30 can comprise a cooling agent. Alternatively, the nanodroplets can be collected in the catchment 30 and cooled at a time after their generation by adding cooling agent to the catchment. Alternatively, the nanodroplets can be collected in the catchment 30 and cooled at a time after their generation by removing the nanodroplets and contacting them with a cooling agent. In one embodiment, a portion of the catchment 30 is removed with the nanodroplets and contacted with a cooling agent.

In some embodiments, the catchment 30 can comprise a container comprising a cooling agent. By way of non-limiting example, the catchment 30 can comprise a container comprising liquid nitrogen. In some embodiments, the container comprised by the catchment 30 can be made of glass, ceramic, plastic, or the like. Any container suitable for containing liquid nitrogen can be used. Containers known in the art and commercially available include Cat No 13982242; PrincetonCryo; Flemington, N.J. In some embodiments, the container comprised by the catchment 30 can be suitable for storing the vitrified biological sample, e.g. the container can comprise a cryogenic storage container. Such containers known in the art and commercially available include Cat No. 366656; ThermoScientific; Rochester, N.Y. In some embodiments, the catchment 30 can be insulated to prevent and/or slow the warming of the cooling agent. In these embodiments, any suitable insulating agent and/or method can be used.

In accordance with some embodiments of the invention, the catchment can be formed by molding, machining, micro-machining, etching, and/or casting into forms that provide the basic elements of the catchment. In some embodiments, the catchment is comprised of portions and/or layers. In some embodiments, adhesives can be used to adhere the portions and/or layers together to form the catchment. Any suitable adhesive can be used to adhere the portions and/or layers together to form the catchment.

In some embodiments, the catchment 30 can comprise a means of collecting the nanodroplets. The collection means can be a film or carrier. Suitable materials for collection means can include, but are not limited to, polyethylene, polyester, polyurethane, polypropylene, and polyvinyl chloride. Preferably, the collection means does not absorb the nanodroplets, i.e. nanodroplets which fall onto the colledtion means substantially retain a spherical shape. In some embodiments, the collection means can be contacted with a cooling agent and then placed in a cryogenic storage container. In some embodiments, the collection means can be placed in a cryogenic storage container comprising a cooling agent. In some embodiments, the collection means can be placed in a cryogenic storage container and the container can then be contacted with a cooling agent.

By way of non-limiting example, the catchment 30 can comprise “blood paper.” Blood paper is a thin sheet of polyethylene material which provides a large surface area to store and retrieve nanodroplets of a biological sample easily. By using the blood paper, nanodroplets can be handled in more automated and high-throughput manner, especially for storage and thawing steps. Blood paper available commercially includes MED5051; Avery; Brea, Calif.

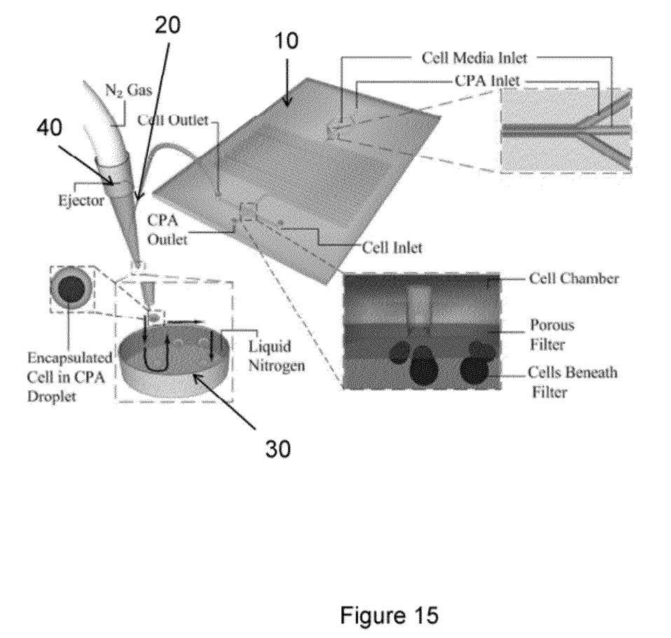

Although described above with respect to a syringe alone, it is contemplated that the reservoir 10 can comprise a microfluidics device. FIG. 15 depicts one embodiment of a system for vitrifying a biological sample as described herein where the reservoir 10, comprises a microfluidics device. In some embodiments, the microfluidics device can be comprised of one or more microchannels. In some embodiments, the microchannels can be separated by a porous membrane.

As used herein, the terms “microfluidics device” and “microchip”, which are used interchangeably herein, refer to a structure or substrate having microfluidics structures contained therein or thereon. In some embodiments, the chip can be detachably connected to a microfluidics system. As used herein, the term “microfluidics system” refers to a machine capable of the manipulation of microliter and/or nanoliter volumes of fluids. As used herein, the term “channel” refers to any capillary, channel, tube, or groove that is deposed within or upon a substrate. A channel can be a microchannel; a channel that is sized for passing through microvolumes of liquid.

In some embodiments, the microfluidics device can optionally have a means for contacting the biological sample or solution comprising the biological sample with a cryoprotective agent (CPA).

In some embodiments, the microfluidics device can have channel dimensions of from 10 um to 1 mm in width, from 10 um to 1 mm in height, and from 1 cm to 2 m in length. In some embodiments, the channel can have a square, rectangular, u-shaped, rounded, or other shape of cross-section.

In some embodiments, the flow rate can be from 0.1 mL/hr to 100 mL/hr. In some embodiments, the flow rate can be about 10 mL/hr. Substructures can be placed in the microchannel to disrupt the flow, prevent shear stress, and/or encourage mixing of the solution comprising the biological sample. In one embodiment, the substructures are herringbone in shape; 25 um wide, 20 um deep, and 25 um apart. In some embodiments, the microchannels of a microfluidics device are serpentine. In some embodiments, the biological sample is contained in a chamber of a first microchannel connected to a second microchannel comprising a solution of CPA by a porous membrane. Such microfluidics devices are described in Song et al. Lab Chip 2009 9:1874-1881; which is incorporated herein in its entirety. In some embodiments, the reservoir 10, comprising a microfluidics device can be directly connected to the nozzle 20.

Any suitable means of generating nanodroplets known in the art can be used. In one embodiment, the nanodroplets are generated by a gas coflow muzzle. One embodiment of a gas coflow muzzle is depicted in FIGS. 17A and 17B. The nozzle 20 is inserted into a gas coflow muzzle 40. In the embodiment depicted in FIGS. 17A and 17B is the nozzle 20 is a needle and the gas coflow muzzle 40 is a 200 uL pipette tip. The nozzle 20 can be inserted through the wall of the gas coflow muzzle 40 and project into tip of the gas coflow muzzle 40. In one embodiment, the nozzle 20 extends approximately 2 mm beyond the tip of the gas coflow muzzle 40. In some embodiments, the nozzle 20 extends from 0 mm to 20 mm beyond the tip of the gas coflow muzzle 40, e.g. 0 mm, 1 mm, 2 mm, 5 mm, 10 mm, 15 mm, or 20 mm beyond the tip of the gas coflow muzzle 40. In one embodiment, the nozzle 20 is inserted through the wall of the gas coflow muzzle 40 at a point 2 cm from the end of the gas coflow muzzle 40. In some embodiments, the nozzle 20, is inserted through the wall of the gas coflow muzzle 40 at least 0.5 cm from the end of the gas coflow muzzle 40, e.g. about 0.5 cm, about 1 cm, about 2 cm, about 4 cm, about 6 cm, about 8 cm, about 10 cm, or further from the end of the gas coflow muzzle 40.

A muzzle 40 can be of plastic, metal, glass and the like, or combinations thereof in the approximate shape of a hollow funnel or cylinder, where the end of the muzzle 40 furthest from the gas source 42 has an opening larger than the nozzle 20 being employed. In some embodiments, the muzzle 40 can be a pipette tip. In some embodiments, the muzzle 40 can be a 200 uL pipette tip. In accordance with some embodiments of the invention, the muzzle can be formed by molding, machining, micro-machining, etching, and/or casting into forms that provide the basic elements of the muzzle. In some embodiments, the muzzle is comprised of portions and/or layers. In some embodiments, adhesives can be used to adhere the portions and/or layers together to form the muzzle. Any suitable adhesive can be used to adhere the portions and/or layers together to form the muzzle.

A flow of gas is provided to the gas coflow muzzle 40 by a gas reservoir 42. In the embodiment shown in FIG. 17A, the gas exits the gas reservoir at a desired flow rate via a gas flow regulator 44. The gas flows through a gas tubing 46 to reach the gas coflow muzzle 40. As the gas leaves the gas coflow muzzle 40 via the tip of the gas coflow muzzle, it passes the terminus of the nozzle 20, generating nanodroplets at the droplet formation point 22.

In some embodiments, the flow of the gas through the gas coflow muzzle 40 can be from 0.1 L/min to 6.0 L/min. In some embodiments, the flow of the gas through the gas coflow muzzle 40 can be from 0.5 L/min to 6.0 L/min. In some embodiments, the flow of the gas through the gas coflow muzzle 40 can be from 0.5 L/min to 1.5 L/min. In some embodiments, the flow of the gas through the gas coflow muzzle 40 can be from 3.0 L/min to 5.0 L/min. In some embodiments, the flow of the gas through the gas coflow muzzle 40 can be from 3.2 L/min to 4.8 L/min. In some embodiments, the flow of the gas through the gas coflow muzzle 40 can be about 3.2 L/min. In some embodiments, the flow of the gas through the gas coflow muzzle 40 can be about 4.0 L/min. In some embodiments, the flow of the gas through the gas coflow muzzle 40 can be about 4.8 L/min.

In some embodiments, a lower gas flow rate is preferred to a higher gas flow rate provided that the lower gas flow rate produces a nanodroplet having a volume less than 500 nL.

In some embodiments, the solution comprising a biological sample is caused to flow through the nozzle 20 into the gas coflow muzzle 40. In some embodiments, the flow of the solution comprising the biological sample is 0.01 mL/min to 3.0 mL/min. In some embodiments, the flow of the solution comprising the biological sample is 0.05 mL/min to 0.9 mL/min. In some embodiments, the flow of the solution comprising the biological sample is 0.1 mL/min to 0.5 mL/min. In some embodiments, the flow of the solution comprising the biological sample is about 0.2 mL/min. In some embodiments, the flow of the solution comprising the biological sample is about 0.8 mL/min.

A muzzle 40 can be connected to a gas source 42 via a gas connection 46. A gas connection can be, for example, tubing. Tubing can be of plastic, metal, and the like or combinations thereof. Tubing can be plastic tubing, e.g. polyethylene tubing. In some embodiments, the tubing is TYGON® tubing. In some embodiments, the tubing is TYGON® tubing with an internal diameter of 3.2 mm. The flow of the gas from a gas source 42 to the muzzle 40 can be controlled by a regulator 44.

The gas of a gas coflow system can be any gas which is not toxic to the biological sample. In some embodiments, the gas can be nitrogen gas, oxygen gas, or mixtures thereof.

In some embodiments, the biological sample comprises red blood cells, and the device comprises a gas coflow muzzle 40. In some embodiments, the rate of flow of the solution comprising the biological sample through the nozzle 20 can be about 0.2 mL/min. In some embodiments, the rate of the gas flow through the gas coflow muzzle 40 can be about 3.2 L/min. In some embodiments, the CPA concentration can be about 2.5 M. In some embodiments, the CPA concentration can be about 2.5 M of glycerol. In some embodiments, the droplet collection distance 32 can be about 60 mm.

In some embodiments, the nanodroplets can be generated via a plunger 50, as shown in FIG. 18. When the plunger 50 is caused to move, it increases pressure upon the biological sample in the reservoir 10, causing the solution comprising the biological sample to flow through the nozzle 20. The plunger 50 can comprise part of the wall of a reservoir 10 or it can act upon one or more flexible walls of the reservoir 10. The plunger 50 can be operated by any manual or automated means. For example, a plunger 50 can be controlled by a solenoid. The use of a solenoid-operated plunger to generate nanodroplets has been described in Song et al. PNAS 2010 107:4596-4600; which is incorporated by reference herein in its entirety. In some embodiments, solenoids can be automated.

In some embodiments, the nanodroplets can be generated via an acoustic drop generator. Briefly, sound waves are caused to move through a solution comprising a biological sample in a nozzle or nozzles, resulting in nanodroplets separating from the solution and falling through the nozzle. It should be noted that when acoustic drop generators are used, the opening of the nozzle 20 does not necessarily determine the size of the droplet. Relatively large diameter nozzles 20 can be employed, such that the drop does not contact the tip of the nozzle 20. In some embodiments, acoustic generators can be used to generate nanodroplets in a device not comprising a nozzle 20, but merely a port 12 through which the solution comprising the biological solution can exit the reservoir 10. Acoustic generation of nanodroplets is known in the art and has been described in detail in Demirci and Montesano. Lab Chip 2007 7:1139-1145; Demirci et al. Ieee Transactions on Semiconductor Manufacturing 2004 17:517-524; Demirci, et al. Ieee Transactions on Semiconductor Manufacturing. 2005 18:709-715; and Demirci. Journal of Microelectromechanical Systems. 2006 15: 957-966; which are incorporated by reference herein in their entirety.

In some embodiments, the nanodroplets can be generated via an inkjet. Suitable, non-limiting examples of inkjets are described, for example in US Patent Publication Nos. US2008/0143782; US2003/0059817; and US2005/0201895; which are incorporated by reference herein their entirety.

In some embodiments, the nanodroplets can be generated by spraying. Spraying can comprise electrospraying, nebulization, electrostatic spraying, and the like. Suitable, non-limiting examples of devices for creating a spray of nanodroplets are described, for example in U.S. Pat. Nos. 7,424,980; 5,572,023 and U.S. Patent Publication Nos. US2008/0038152; US2010/0155496; and US2009/0152371 which are incorporated by reference herein their entirety.

In some embodiments, control of gas flow regulator 44, platform 24 and reservoir 10 can be automated. In an embodiment in which control of the flow of solution from reservoir 10 is automated, a solenoid can be used, as discussed above. In other embodiments, one or more computing devices or systems may be used to control gas flow regulator 44, platform 24 and reservoir 10. For example, a computing device may be used in conjunction with gas flow regulator 44 to select and regulate gas pressure from gas reservoir 42. Alternatively or additionally, a computing device may be coupled to reservoir 10, port or opening 12 and/or nozzle 20 in order to control the flow of solution from reservoir 10. Alternatively or additionally, a computing device may be coupled to platform 24 to automate movement of platform 24. For example, a computing device may be used to change droplet collection distance 32 and/or to change the position and orientation of nozzle 20 and/or gas coflow muzzle 40.

In some embodiments, one or more sensors may be coupled to the computing device in order to determine the appropriate action to be taken. For example, a temperature sensor (not shown) may be positioned proximate to droplet formation port 22, nozzle 20 and/or gas coflow muzzle 40. If the temperature at nozzle 20 approaches freezing, for example, the computing device can increase the droplet collection distance 32, so as to increase separation between nozzle 20 and the cooling agent (e.g. liquid nitrogen).

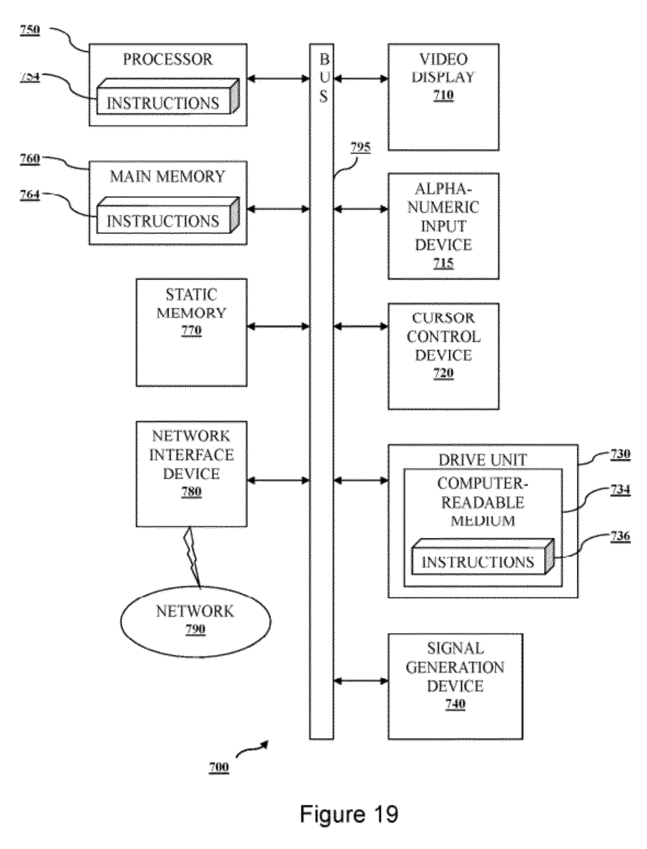

FIG. 19 shows a diagrammatic representation of machine in the exemplary form of computer system 700 within which a set of instructions, for causing the machine to perform such control of gas flow regulator 44, platform 24 and reservoir 10 discussed herein, may be executed. In alternative embodiments, the machine operates as a standalone device or may be connected (e.g., networked) to other machines. The machine may comprise a personal computer (PC), a tablet, a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

According to some embodiments, computer system 700 comprises processor 750 (e.g., a central processing unit (CPU), a graphics processing unit (GPU) or both), main memory 760 (e.g., read only memory (ROM), flash memory, dynamic random access memory (DRAM) such as synchronous DRAM (SDRAM) or Rambus DRAM (RDRAM), etc.) and/or static memory 770 (e.g., flash memory, static random access memory (SRAM), etc.), which communicate with each other via bus 795.

According to some embodiments, computer system 700 may further comprise video display unit 710 (e.g., a liquid crystal-display (LCD), a light-emitting diode display (LED), an electroluminescent display (ELD), plasma display panels (PDP), an organic light-emitting diode display (OLED), a surface-conduction electron-emitted display (SED), a nanocrystal display, a 3D display, or a cathode ray tube (CRT)). According to some embodiments, computer system 700 also may comprise alphanumeric input device 715 (e.g., a keyboard), cursor control device 720 (e.g., a mouse or controller), disk drive unit 730, signal generation device 740 (e.g., a speaker), and/or network interface device 780.

Disk drive unit 730 includes computer-readable medium 734 on which is stored one or more sets of instructions (e.g., software 736) embodying any one or more of the methodologies or functions described herein. Software 736 may also reside, completely or at least partially, within main memory 760 and/or within processor 750 during execution thereof by computer system 700, main memory 760 and processor 750. Processor 750 and main memory 760 can also constitute computer-readable media having instructions 754 and 764, respectively. Software 736 may further be transmitted or received over network 790 via network interface device 780.

While computer-readable medium 734 is shown in an exemplary embodiment to be a single medium, the term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing, encoding or carrying a set of instructions for execution by the machine and that cause the machine to perform any one or more of the methodologies of the disclosed embodiments. The term “computer-readable medium” shall accordingly be taken to include, but not be limited to, solid-state memories, and optical and magnetic media.

It should be understood that processes and techniques described herein with respect to automated control of gas flow regulator 44, platform 24 and reservoir 10 are not inherently related to any particular apparatus and may be implemented by any suitable combination of components. Further, various types of general purpose devices may be used in accordance with the teachings described herein. It may also prove advantageous to construct a specialized apparatus to perform the functions described herein. Those skilled in the art will appreciate that many different combinations of hardware, software, and firmware will be suitable for practicing the disclosed embodiments.

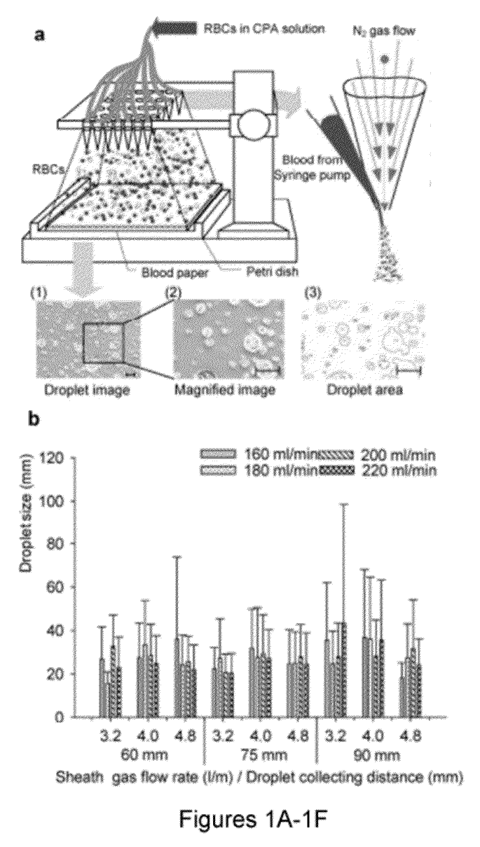

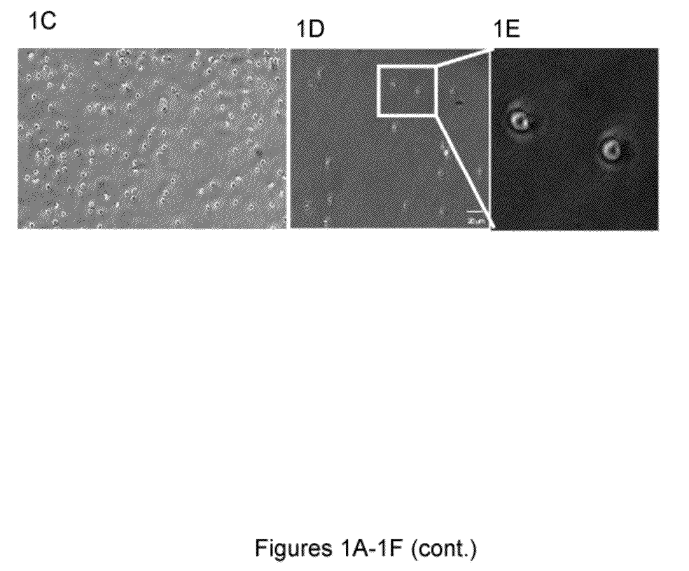

The systems and methods described herein can be used in a high-throughput manner. In some embodiments, high-throughput can refer to a system as described herein capable of preparing a unit of blood for vitrification and/or vitrifying a unit of blood in as little as 10 minutes from the time at which a unit of blood is provided to the reservoir 10 of the system. For example, a high-throughput embodiment of the gas coflow ejector device is depicted in FIG. 1A. In this embodiment, the biological sample is provided to twenty-five nozzles 20, each coupled to an individual gas coflow muzzle 40 in the same platform 24. In the embodiment illustrated in FIG. 1A, nanodroplets are collected by a catchment 30 comprising a single piece of blood paper positioned on a petri dish.

In some embodiments, a high-throughput device can comprise one or more nozzles 20, e.g. 1 nozzle, 2 nozzles, 5 nozzles, 10 nozzles, 25 nozzles, 50 nozzles, 100 nozzles, 200 nozzles, or more nozzles. In some embodiments, a plurality of nozzles can be connected to a single reservoir 10. The plurality of nozzles 20 can be connected directly to reservoir 10 or indirectly via tubing.

In some embodiments, a high-throughput device can comprise one or more reservoirs 10, e.g. 1 reservoir, 2 reservoirs, 5 reservoirs, 10 reservoirs, 25 reservoirs, 50 reservoirs, 100 reservoirs, 200 reservoirs, or more reservoirs. Each reservoir 10 can be connected to one or more nozzles 20. By way of non-limiting example, a high-throughput device can comprise 25 nozzles 20, each connected to a separate reservoir 10. Alternatively, a high-throughput device can comprise twenty-five nozzles 20 and five reservoirs 10, with each reservoir 10 connected to five nozzles 20. Alternatively, a high-throughput device can comprise 25 nozzles 20, all connected to a single reservoir 20.

A high-throughput device can comprise one or more catchments 30, e.g. 1 catchment, 2 catchments, 5 catchments, 10 catchments, or more catchments. Each catchment 30 can receive nanodroplets produced from one or more nozzles 20.

A high-throughput device can comprise one or more platforms 24, e.g. one platform, 2 platforms, 5 platforms, 10 platforms or more platforms. Each platform 24 can support one or a plurality of nozzles 20.

A high-throughput device can comprise any means of droplet generation known to one of ordinary skill in the art and/or discussed herein, e.g. a gas coflow muzzle means of droplet generation or a plunger means of droplet generation.

As used herein, a “biological sample” refers to a sample comprising tissues, cells, biological fluids, polypeptides, nucleic acids, or other biological substances. In some embodiments a biological sample can further comprise preservatives. In some embodiments, a sample can be obtained from a subject. In some embodiments a sample can be a diagnostic sample obtained from a subject. By way of non-limiting example, a sample can be a gamete, sperm, eggs, an embryo, a zygote, chondrocytes, red blood cells, blood, portions or fractions of blood, hepatic cells, fibroblasts, stem cells, cord blood cells, adult stem cells, induced pluripotent stem cells, autologous cells, autologous stem cells, bone marrow cells, hematopoietic cells, hematopoietic stem cells, somatic cells, germ line cells, differentiated cells, somatic stem cells, embryonic stem cells, serum, plasma, sputum, cerebrospinal fluid, urine, tears, alveolar isolates, pleural fluid, pericardial fluid, cyst fluid, tumor tissue, a biopsy, saliva, an aspirate, or combinations thereof. In some embodiments, a sample can be obtained by resection, biopsy, or egg retrieval.

As used herein “hematopoietic cell” refers to any differentiated, progenitor, or stem cell which is a component of blood or gives rise to a cell which is a component of blood.

As used herein “cord blood cell” refers to any cell present in the blood comprising an umbilical cord or placenta. In some embodiments, the umbilical cord or placenta is that of a newborn. Methods for obtaining cord blood are described, for example in U.S. Patent Publication 2010/0189696; which is incorporated by reference herein in its entirety.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cell forming the body of an organism, as opposed to a germ line cell.

The terms “stem cell” or “undifferentiated cell” as used herein, refer to a cell in an undifferentiated or partially differentiated state that has the property of self-renewal and has the developmental potential to differentiate into multiple cell types, without a specific implied meaning regarding developmental potential (i.e., totipotent, pluripotent, multipotent, etc.). A stem cell is capable of proliferation and giving rise to more such stem cells while maintaining its developmental potential. In theory, self-renewal can occur by either of two major mechanisms. Stem cells can divide asymmetrically, which is known as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. While each of these multipotent cells may be considered stem cells, the range of cell types each such stem cell can give rise to, i.e., their “developmental potential,” can vary considerably. Alternatively, some of the stem cells in a population can divide symmetrically into two stem cells, known as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

The term “somatic stem cell” is used herein to refer to any pluripotent or multipotent stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, neural stem cells, neural crest stem cells, mesenchymal stem cells, hematopoietic stem cells, and pancreatic stem cells.

As used herein “induced pluripotent stem cell” or “iPSC” or “iPS cell”, which are used interchangeably herein refer to pluripotent cells derived from differentiated cells. For example, iPSCs can be obtained by overexpression of transcription factors such as Oct4, Sox2, c-Myc and Klf4 according to the methods described in Takahashi et al. (Cell, 126: 663-676, 2006). Other methods for producing iPSCs are described, for example, in Takahashi et al. Cell, 131: 861-872, 2007 and Nakagawa et al. Nat. Biotechnol. 26: 101-106, 2008; which are incorporated by reference herein in their entirety.

In some embodiments, the biological sample comprises blood or blood cells. In some embodiments, the biological sample comprises whole blood or a fraction of whole blood, e.g. plasma (platelet-rich or platelet-poor plasma), platelets, or red blood cells or a combination thereof.

In some embodiments, the biological sample is collected from a subject. In some embodiments, the biological sample can be can be freshly collected. In some embodiments, the sample can be stored prior to being vitrified according to the methods and systems described herein. In some embodiments, the sample is an untreated sample. As used herein, “untreated sample” refers to a biological sample that has not had any prior sample pre-treatment except for dilution and/or suspension in a solution. Exemplary methods for treating a sample include, but are not limited to, centrifugation, filtration, sonication, homogenization, heating, and any combination thereof.

In some embodiments, a sample can be obtained from a subject and preserved or processed prior to being utilized in the methods and compositions described herein. By way of non-limiting example, a sample can be refrigerated. In some embodiments, the sample can be a processed or treated sample. Exemplary methods for treating or processing a sample include, but are not limited to, centrifugation, filtration, sonication, homogenization, heating, contacting with a preservative (e.g. anti-coagulant or nuclease inhibitor) and any combination thereof. In some embodiments, the sample can be treated with a chemical and/or biological reagent. Chemical and/or biological reagents can be employed to protect and/or maintain the stability of the sample during processing and/or storage. By way of non-limiting example, a blood sample can be treated with an anti-coagulant prior to being utilized in the methods and compositions described herein. The skilled artisan is well aware of methods and processes for processing, preservation, or treatment of samples.

By way of non-limiting example, a solution of red blood cells to be vitrified can be prepared as follows: a buffy coat sample can mixed with 3.5 ml of Citrate Phosphate Dextrose Adenine (CPDA-1) anticoagulant for 2 min at 20° C., and then centrifuged at 2000 rpm for 10 min at 20° C. (Allegra 6 Centrifuge, Beckman Coulter, USA). For each 100 ml of CPDA-1, 327 mg citric acid, 2.635 g sodium citrate, 222 mg monobasic sodium phosphate, 3.175 g dextrose, 27 mg adenine can be used.

By way of non-limiting example, a solution of murine oocytes to be vitrified can be prepared as follows: mice can be superovulated by intraperitoneal injection of 5 IU of pregnant mare serum gonadotropin (EMD Chemicals, NJ, USA) followed by the injection of 5 IU human chorionic gonadotrophin 48 h later (EMD Chemicals). A total of 15 h after human chorionic gonadotrophin treatment, mice are euthanized. A small incision can be made over the midsection, the skin reflected back, and the peritoneum entered with a sharp dissection to expose the viscera. The oviducts can be immediately excised and placed into a culture dish containing 4 ml EMBRYOMAX® flushing hold medium (FHM) 4-(2-hydroxyethyl)-1-piperazineethanesulfonic acid (HEPES) buffered medium (Millipore Corporation, Billerica, M A, USA) supplemented with 4 mg/ml bovine serum albumin (BSA). Oviducts can then be transferred to a center-well dish containing 1 ml FHM medium supplemented with 4 mg/ml BSA for cumulus-oocyte complex collection. While holding the oviduct in place with forceps, an incision can be made in the swollen part of the oviduct using an insulin needle to allow cumulus-oocyte complex extrusion. Oocytes can then be transferred to a center-well dish filled with 1 ml hyaluronidase solution (0.3 mg/ml; Sigma, Mo., USA) in FHM medium until the cumulus cells were dispersed, followed by five washes in 50 μl of FHM medium drops. Oocytes can be collected in 50 μl drops of potassium simplex optimized medium (KSOM) medium (Millipore Corporation, MA, USA), covered with mineral oil (Sigma, Mo., USA) and cultured at 37° C. in 5% CO₂ and 95% air.

In some embodiments, the biological sample is present in the reservoir 10 in a solution. The solution can comprise the biological sample and one or more types of media, buffers, nutrients, and/or antibiotics. In some embodiments the solution can comprise cell culture media. Appropriate types and concentrations of solutions for use with particular types of biological samples are well known in the art. Non-limiting examples of a solution include 2.5 M glycerol, FHM medium, FHM medium+1 M sucrose+4 mg/mL BSA, FHM medium+0.5 M sucrose+4 mg/mL BSA, FHM medium+0.2 M sucrose+4 mg/mL BSA, saline solution, 0.9% saline, 0.9% saline+0.2% glucose, 1% saline, 0.5% saline, 0.3% saline, 0.2% saline, and KSOM medium. In some embodiments, the biological sample comprises oocytes and the solution comprises KSOM medium.

In some embodiments, the solution comprising a biological sample can further comprise one or more cryoprotective agents. As used herein, the phrase “cryoprotective agent” refers to a chemical or a chemical solution which facilitates the process of cryoprotection by reducing the injury of cells during freezing and thawing. Preferably, the cryoprotective agent is non-toxic to the cellular matter under the conditions at which it is used (i.e. at a particular concentration, for a particular exposure time and to cells in a medium of a particular osmolality). A cryoprotective agent may be cell permeating or non-permeating. Examples of cryoprotective agents include but are not limited to, dehydrating agents, osmotic agents and vitrification solutes (i.e., solutes that aid in the transformation of a solution to a glass rather than a crystalline solid when exposed to low temperatures). In some embodiments, a CPA can be a naturally-occurring CPA such as ectoin.

Non-limiting examples of CPAs include SrCl₂, sucrose, trehalose, glycerol, polymers, polyvinylpyrrolidone, polyvinyl alcohol, dimethylsulphoxide (DMSO), 1,2-propanediol (PROH), ethylene glycol (EG), mannitol, hydroxyethyl starch (HES), monosaccharide and sugar alcohols; ectoin; methylcellulose; and polyethylene glycol (PEG).

Further non-limiting examples of cryprotective agents are described in U.S. Pat. Nos. 5,071,741; 7,112,576; 5,897,987; and U.S. Patent Publication Nos. US2008/0254439; US2009/0029340; and US2011/0250581 which are incorporated by reference herein in their entirety.

In some embodiments, a CPA can be present in a solution comprising a biological sample at from 1 mM to 6 M. In some embodiments, a CPA is present in a solution comprising a biological sample at less than 6 M, e.g. less than 5M, less than 4 M, less than 3 M, less than 2M, less than 1M, less than 500 mM or lower. In some embodiments a CPA is present in a solution comprising a biological sample at about 3 M or lower. In some embodiments a CPA is present in a solution comprising a biological sample at about 2 M or lower.

In some embodiments, a CPA is added to the solution comprising a biological sample prior to the biological sample and/or solution comprising a biological sample being vitrified. In some embodiments, a CPA is added to the solution comprising a biological sample prior to the biological sample and/or solution comprising a biological sample exiting the nozzle 20 as a nanodroplet. In some embodiments, a CPA is added to the solution (also referred to herein as “CPA loading”) comprising a biological sample prior to the solution being added to the reservoir 10. In some embodiments, a CPA is added to the solution comprising a biological sample while the solution is in the reservoir 10.

In some embodiments, a CPA is added to the solution comprising a biological sample in one step. In some embodiments, a CPA is added to the solution comprising a biological sample in multiple steps, i.e. step-wise addition. Step-wise addition can minimize osmotic shock and reduce damage to the biological sample. In some embodiments, a CPA is added by step-wise addition in 2 or more steps, i.e. in 2 steps, in 3 steps, in 4 steps, in 5 steps, in 6 steps, or more steps. In some embodiments, the first step of a step-wise addition results in a concentration of CPA of less than ½ of the final desired concentration. In some embodiments, the first step of a step-wise addition results in a concentration of CPA of less than ¼ of the final desired concentration.

In some embodiments, step-wise addition can comprise moving a biological sample from a first solution to a second solution having a greater CPA concentration than the first solution. In some embodiments, step-wise addition can comprise adding multiple aliquots concentrated CPA to a solution comprising a biological sample over time.

In some embodiments, a CPA can be added using a microfluidics device. In some embodiments, the microfluidics device is comprised by the reservoir 10. Examples of microfluidics devices for the step-wise addition of CPA are described in detail in Song et al. Lab Chip 2009 9:1874-1881; which is incorporated herein in its entirety. In some embodiments, the microfluidics device is designed such that a flow of CPA is mixed with a flow of a solution comprising a biological sample at a desired rate. In some embodiments, the microfluidics device is designed so that multiple flows of CPA are progressively added to a flow of a solution comprising a biological sample, thereby increasing the CPA concentration in the solution comprising the biological sample at a desired rate. In some embodiments, the microfluidics device is designed to comprise at least two microchannels separated by a porous membrane. The biological sample is in the first channel while a flow of CPA is in the second channel. The concentration of the CPA in the second channel can be increased over time and diffuse through the membrane. In some embodiments, the CPA and the biological sample are introduced to the microfluidics device via separate ports.

In some embodiments, the total time required to add a CPA to a solution comprising a biological sample is less than 20 minutes, e.g. 18 minutes, 15 minutes, 12 minutes, 10 minutes, 5 minutes or less. In some embodiments, the total time required to add a CPA to a solution comprising a biological sample is less than 10 minutes.

In some embodiments, the solution comprising a biological sample and/or the biological sample can further comprise a hydrogel. As used herein, “hydrogel” refers to a water-containing polymer network formed by polymers which expand in volume upon hydration. Hydrogels are well known in the art. Non-limiting examples of biocompatible materials suitable for use in a hydrogel include, alginates, chitosan, collagen, hyaluronate, fibrin, hydroxypropyl methylcellulose (HPMC, e.g., METHOCELTM, etc.), alginate, sodium alginate, cellulose hydrogel, polyvinylpyrrolidone, hydroxypropyl cellulose (HPC; e.g., KLUCELTM, etc.), nitrocellulose, hydroxypropyl ethylcellulose, hydroxypropyl butylcellulose, hydroxypropyl pentylcellulose, methyl cellulose, hydroxyethyl cellulose, alkyl celluloses, hydroxyalkyl celluloses, cellulose ethers, cellulose acetate, carboxymethyl cellulose, sodium carboxymethyl cellulose, calcium carboxymethyl cellulose, poly-hydroxyalkyl methacrylate, polymethacrylic acid, polymethylmethacrylate, poly vinyl alcohol, sodium polyacrylic acid, calcium polyacrylic acid, polyacrylic acid, acidic carboxy polymers, carboxypolymethylene, carboxyvinyl polymers, carboxymethylamide, polyhyaluronic acid, polypeptide, elastin, polylactic acid, polyglycolic acid, chitin, polyethylene oxide, polyethylene glycol, polyvinyl alcohol, polyacrylic acid, polyacrylamide, poly (N-vinyl-2-pyrrolidone), polyurethane, and polyacrylonitrile. In some embodiments, the solution comprising a biological sample and/or the biological sample further comprises both a hydrogel and a cryoprotective agent.

In some embodiments, the flow rate of a solution comprising a biological sample in a microfluidics device can be from about 0.1 uL/min to 1 mL/min. In some embodiments the flow rate of a solution comprising a biological sample in a microfluidics device can be from about 1 uL/min to 100 uL/min. In some embodiments the flow rate of a solution comprising a biological sample in a microfluidics device can be from about 1 uL/min to 50 uL/min. In some embodiments, the solution comprises red blood cells and the flow rate is about 10 uL/min.

In some embodiments, the flow rate of a CPA solution in a microfluidics device can be about the same as the flow rate of the solution comprising a biological sample. In some embodiments, the flow rate of a CPA solution in a microfluidics device can be lower than the flow rate of the solution comprising a biological sample. In some embodiments, the flow rate of a CPA solution in a microfluidics device can be higher than the flow rate of the solution comprising a biological sample.

In some embodiments, the catchment 30 can comprise a cooling agent or the nanodroplets collected in the catchment 30 can be contacted with a cooling agent. In some embodiments, the cooling agent can be a substance which is maintained at a temperature of less than −130° C. The cooling agent is preferably non-toxic or of low toxicity to the biological sample being vitrified. By way of non-limiting example, a cooling agent can be liquid nitrogen, nitrogen vapor, liquid helium, and/or helium vapor.

In some embodiments, the nanodroplet directly contacts the cooling agent. In some embodiments, the entire surface of the nanodroplet is capable of contacting the cooling agent, e.g. the nanodroplet is not bound to and/or adhered to a substrate or carrier. In some embodiments, the systems and methods described herein relate to carrier-free vitrification. In some embodiments, the nanodroplets are not directly contacted with a cooling agent.

For clinical use of the systems and methods described herein, sterility is a desirable characteristic. Accordingly, provided herein are means of practicing the methods and systems of the invention in sterile conditions.

In some embodiments, the entire system is operated in a sterile room and/or in a sterile hood. A biological sample can be provided in a container that can optionally be sterilized on the outside surface or at the location where the biological sample will exit the container (e.g. a standard blood collection bag with sterile ports).

In some embodiments, the entire system is a closed system, i.e. once the biological sample enters the system at a port of the reservoir; the sample is contained within the device and does not contact the unsterile atmosphere until at least the point at which it has been vitrified.

In some embodiments, components of the systems described herein, e.g. nozzles 20, ejectors, reservoirs 10, tubing connecting nozzles 20 and reservoirs 10, gas coflow muzzles 40, and gas connectors 36 can be sterilized by, for example, autoclaving and/or flowing ethanol through them. Alternatively, components of the systems described herein may be disposable.

The cooling agent can be sterilized by methods well known in the art, e.g. liquid nitrogen can be sterilized using sterile polytetrafluoroethylene (PTFE) cartridge filtration; or ultra-violet radiation (see McBurnie and Bardo. Pharm Technol North America 2002 26:9; Parmegiani et al. Hum Reprod 2009 24:2969; and Bielanski and Vajta. Hum Reprod 2009 24:2457-2467; which are incorporated by reference herein in their entirety.

In the methods and systems described herein, following vitrification, the biological sample can be stored. In some embodiments, the vitrified biological sample is cryogenically stored. In some embodiments, the vitrified biological sample is stored at a temperature equal to or lower than −130° C. Cryogenic storage freezers and containers (e.g. cryovials and ampoules) are known in the art. Cryogenic storage containers are commercially available in sterile forms (e.g. Cat No. 366656; ThermoScientific; Rochester, N.Y.). In some embodiments, the vitrified biological sample is “banked” for future use in the subject from which it originated or a second subject. In some embodiments, the vitrified biological sample is stored for future diagnostic use.

In some embodiments of the methods described herein, after the biological sample has been vitrified, the sample can be thawed at a later time. The biological sample can be thawed minutes, hours, days, weeks, months, or years after being vitrified, e.g. a vitrified biological sample can be thawed 1 hour, or 1 day, or 1 week, or 1 month, or 1 year or 10 years; or longer after vitrification.

In preferred embodiments, the thawing of the vitrified biological sample occurs rapidly. In some embodiments, the process of thawing occurs along with the removal of CPAs (also called CPA unloading herein).

In some embodiments, rapid warming and CPA unloading comprises contacting the vitrified nanodroplets with a warm thawing solution. In some embodiments, the warm thawing solution can be from 20° C. to 44° C. in temperature. In some embodiments, the warm thawing solution can be from 22° C. to 27° C. In some embodiments, the warm thawing solution can be from 34° C. to 40° C. In some embodiments, the warm thawing solution can be about 25° C. In some embodiments, the warm thawing solution can be about 37° C.

In some embodiments, the vitrified nanodroplets are kept in contact with a cooling agent until immediately prior to being contacted with a warm thawing solution.

In some embodiments, the warm thawing solution can comprise one or more types of medium, buffers, nutrients, and/or antibiotics. In some embodiments the warm thawing solution can comprise cell culture media. Appropriate types and concentrations of solutions for use with particular types of biological samples are well known in the art. Non-limiting examples of a warm solution include 2.5 M glycerol, FHM medium, FHM medium+1.M sucrose+4 mg/mL BSA, FHM medium+0.5 M sucrose+4 mg/mL BSA, FHM medium+0:2 M sucrose+4 mg/mL BSA, saline solution, 0.9% saline, 0.9% saline+0.2% glucose, 1% saline, 0.5% saline, 0.3% saline, 0.2% saline, and KSOM medium.

In some embodiments, the nanodroplets are kept in contact with the warm thawing solution for at least 30 seconds, e.g. for 30 seconds, for 1 minute, for 2 minutes, for 5 minutes, for 10 minutes or longer.

In some embodiments, the nanodroplets are not in direct contact with the warm thawing solution, e.g. the nanodroplets are in a container which is contacted with a warm thawing solution.

In some embodiments, the warm thawing solution can comprise a concentration of CPA lower than that present in the nanodroplets. In some embodiments, CPA unloading occurs in a stepwise fashion, e.g. the vitrified nanodroplets are thawed in a first warm thawing solution and then contacted with at least a second warm thawing solution having a lower CPA concentration than the first warm thawing solution.

The volume of the warm thawing solution should be significantly greater than the total volume of the vitrified nanodroplets to be thawed, e.g. at least 2 times greater, at least 5 times greater, at least 10 times greater, at least 20 times greater, at least 100 times greater, or greater than the total volume of the vitrified nanodroplets to be thawed.

In some embodiments, the sample is thawed and/or subjected to CPA removal using a microfluidics device. The microfluidics device can be as described above and instead of flowing solutions of CPA through the device, solutions of media and/or buffers are flowed through the microfluidics device.

In some embodiments, the one or more warm thawing solutions are sterile solutions. In some embodiments, the thawing of the vitrified nanodroplets is conducted under sterile conditions, e.g. in a sterile hood.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting.

### EXAMPLES

### Example 1

### Blood Banking and Vitrification

Blood banking has a broad public health impact influencing millions of lives daily. It could potentially benefit from emerging biopreservation technologies. However, although vitrification has shown advantages over traditional cryopreservation techniques, it has not been incorporated into transfusion medicine mainly due to throughput challenges. Described herein is a scalable method that can vitrify red blood cells in nanodroplets. This approach enables the vitrification of large volumes of blood in a short amount of time, and makes it a viable and scalable biotechnology tool for blood cryopreservation.

Blood shortages pose a major global health challenge that frequently occur during natural disasters, military conflicts, and in clinical settings due to fluctuations in supply and demand [1]. Long-term cryopreservation of blood products provides a supplementary inventory to help meet the demand during such shortages by freezing excess blood. Although the use of additive preservatives has extended the liquid storage of blood products to several weeks (i.e., 42 days for red blood cells (RBCs) [2], [3], [4]), the limited shelf life makes it difficult to manage blood inventories resulting in a large waste [5]. For instance, in 2006, 1.2 million units of blood were discarded in the US alone [6], [7]. New technologies can potentially revolutionize how blood is handled in war and global disaster zones, prevent waste, and reduce vulnerability to shortages. Over the last century, significant progress has been made in understanding the basic factors leading to cryoinjury in RBCs and in development of effective techniques to prevent it [5], [8].

Two major clinical RBC cryopreservation approaches have been established: the high glycerol/slow freezing [9], [10] and the low glycerol/rapid freezing [11], [12], [13] techniques. The high glycerol/slow freezing technique uses 40% (w/v) glycerol with a cooling rate of ˜1° C./min and storage at −80° C. The low glycerol/rapid freezing approach uses 15-20% glycerol with rapid cooling rates (60-120° C./min) by immersing samples in freezing containers into liquid nitrogen (−196° C.) or nitrogen vapor (−165° C.) [1]. However, although both RBC cryopreservation methods are considered effective, cryoinjury to RBCs still occurs during the cooling and warming processes as a result of cell shrinkage [14], [15], toxicity due to the increasing concentrations of solutes [16], [17], [18] during slow freezing, and intracellular ice formation (IIF) during rapid freezing [19].

In contrast, vitrification as a cryopreservation method has provided a means to significantly reduce the damage to various cells and tissues [20], [21], since ice crystal formation and the corresponding intra and extracellular solute accumulation are prevented. Despite the potential advantages of vitrification, its broad application to RBC biopreservation hasn't yet been achieved. Challenges include difficulty to achieve extreme cooling and thawing rates, toxicity of high cryoprotectant agent (CPA) concentrations needed to vitrify suspensions at a realizable temperature, devitrification upon rewarming, and inability to accommodate freezing of large volumes of blood using microliter cryovials. Described herein is a high throughput ultra-rapid vitrification method using cell encapsulating droplets, which overcomes some of the limitations by lowering the required CPA concentrations and achieving ultra-rapid cooling rates via vitrifying RBCs encapsulated in small droplet volumes. Furthermore, RBCs can be stored in liquid nitrogen directly on the collection film. This is the first time that a scalable vitrification method has been introduced to blood cryopreservation. Such technologies have other broad applications in multiple fields, including film boiling [22], spray cooling during the heat treatment of metallic alloys, turbine engines, and nuclear reactors [23].

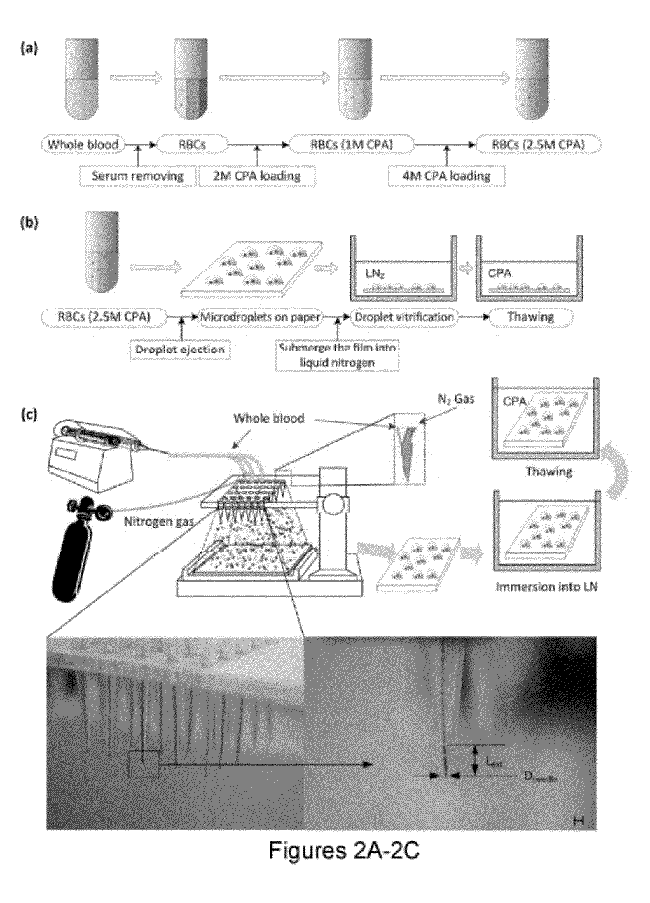

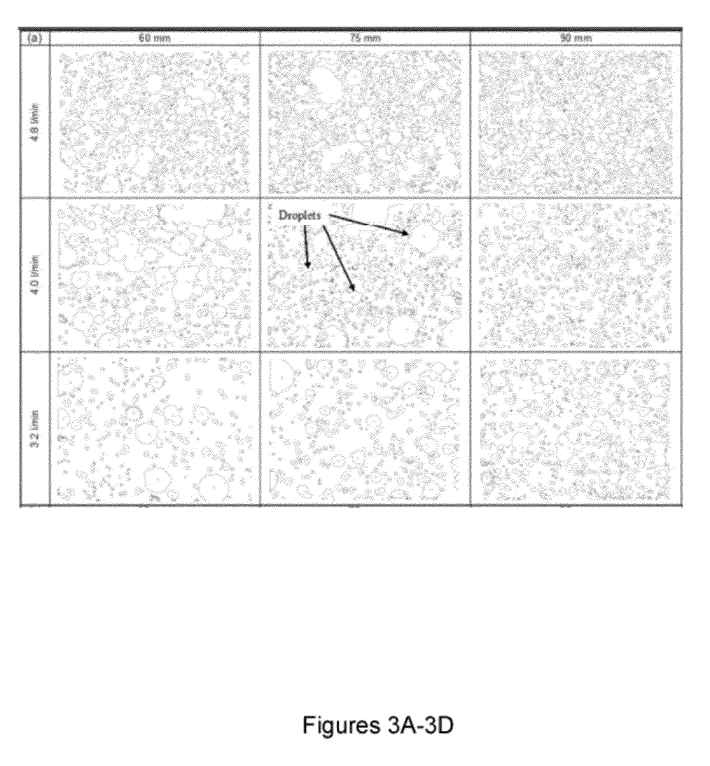

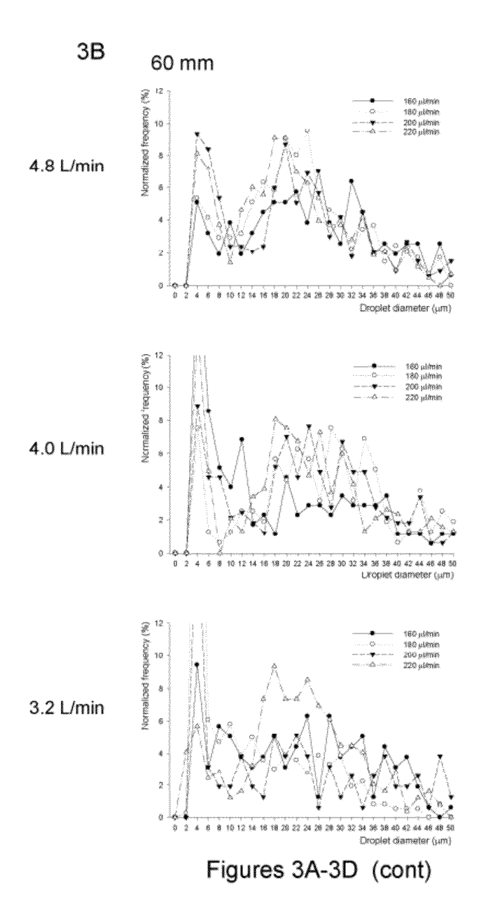

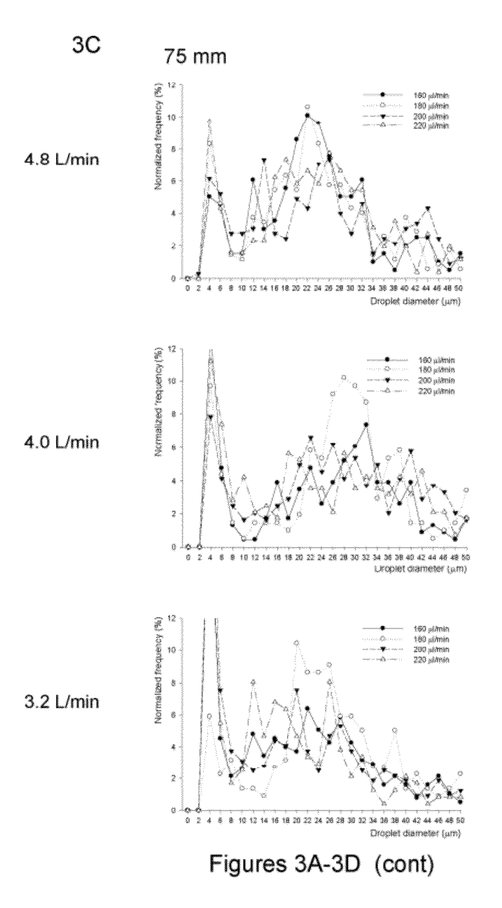

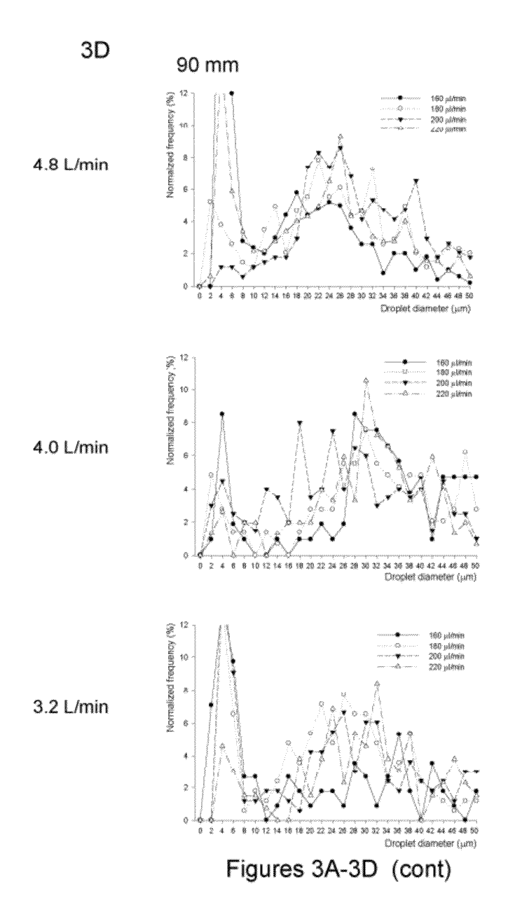

It has been shown that the degree of crystallization during ultra-rapid cooling increases with an increase in droplet radius, especially when the dimensionless radii (r*) are above 0.1 (100 μm) [24]. Therefore, a co-flow ejection system was designed to generate RBC encapsulating nanodroplets (<100 μm) which can then be vitrified at high throughput. Three major parameters affecting the droplet size were investigated, including the droplet collection distance (from ejector tip to droplet collection film), the flow rate of nitrogen gas and the flow rate of CPA loaded RBCs. Droplets generated at different nitrogen flow rates (3.2-4.8 l/min), ejection collecting distances (60-90 mm) were deposited on collection films (FIG. 1A) and the droplet size distributions were analyzed (FIG. 1B). A decrease in droplet size was observed when either the nitrogen gas flow rate or ejection collecting distance was increased (FIG. 1B). However, an increase in flow rate of CPA loaded RBC solution did not result in a significant change in droplet size due to the dominant effect of nitrogen gas flow rate (FIG. 1B, FIG. 2B, FIG. 2C, and FIGS. 3A-3B) within the range from 3.2 to 4.8 l/min over the droplet size. By controlling the nitrogen gas flow rate, droplet diameters were maintained below 100 μm to ensure effective vitrification at a low CPA concentration (2.5 M glycerol, equivalent to a concentration of 23%, Table 1) where the system was operated, minimizing possible toxic and osmotic effects [9], [10].

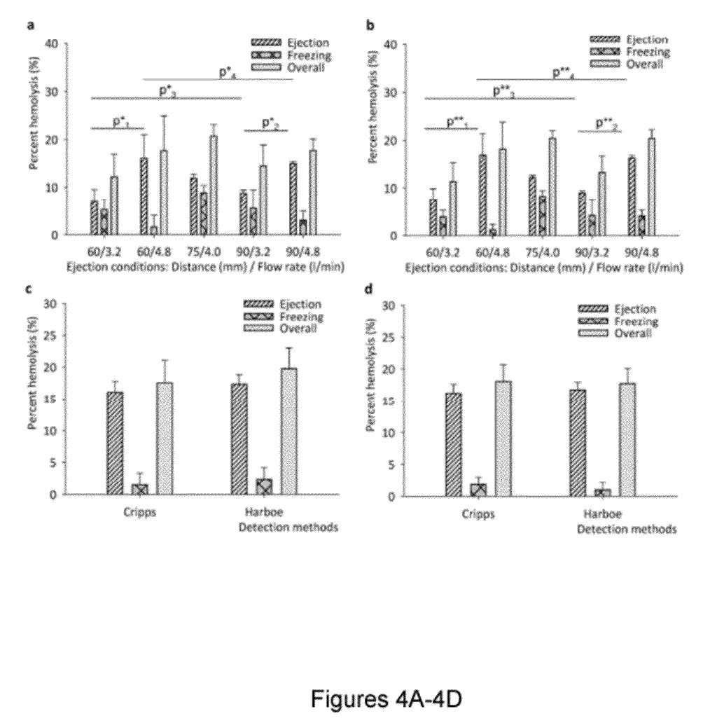

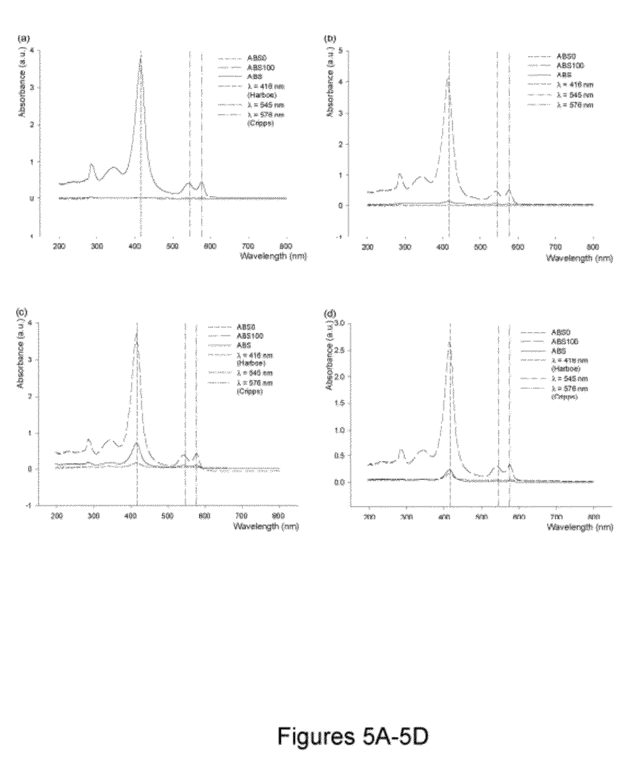

To assess overall hemolysis, the percent hemolysis from each procedure step was evaluated, including CPA loading, ejection, RBC droplet collection on films, and freezing/thawing at five different experimental conditions (FIGS. 4A-4B). The percent hemolysis of each step was calculated using Eqn. 1 with the absorbance values determined using Cripps and Harboe methods as shown in Table 2 and FIGS. 5A-5D. Percent hemolysis due to freezing/thawing and shear stress during ejection ranged from 2 to 8% and 5 to 17% across all experimental conditions, respectively (FIG. 4A-4D). The CPA loading steps led to a smaller percent hemolysis (2% out of 20% total, Table 2) compared to the ejection and vitrification/thawing steps. The percent hemolysis by the collection film was negative due to the possible adhesion of free hemoglobin to the film.

The results showed that an increase in droplet collecting distance from 60 mm to 90 mm did not change percent hemolysis either during ejection or freezing/thawing processes at constant gas flow rates of 3.2 l/min and 4.8 l/min, (FIGS. 4A-4B). However, keeping the ejection distance constant, when nitrogen gas flow rate was increased from 3.2 l/min to 4.8 l/min, a statistically significant increase in percent hemolysis was observed at ejection distances of 60 mm (increase from 6.9% to 16.16%) and 90 mm (increase from 8.8% to 14.7%) (Table 3, and for statistical analysis, Table 6). These observations indicated that the ejector gas flow rate affects RBC hemolysis (Kruskal-Wallis non-parametric analysis of variance, p<0.05) and damage to cells during droplet generation and encapsulation can be minimized by changing the gas flow rate. For the vitrification and thawing steps, an increase in nitrogen flow rate did not have a significant effect on RBC hemolysis (FIGS. 4A-4B). since the average droplet diameter was below 100 μm at each flow rate (FIG. 1B). The combined effects of ejection, freezing, and thawing at the lowest nitrogen flow rate (3.2 l/min) resulted in the lowest percent hemolysis (11%) with minimal dependence on ejection distance

To demonstrate the scalability of the system, droplet-based RBC vitrification experiments were performed using arrays of 4 and 25 independent ejectors activated simultaneously (FIG. 2C). The vitrification was performed at an ejection distance of 60 mm, CPA loaded RBC flow rate of 0.2 ml/min for each ejector and nitrogen gas flow rate of 3.2 l/min. With this 25-ejector setup, in 5 minutes, the system vitrified 25 ml of RBCs loaded with 2.5 M glycerol (1:1, v/v). As shown in FIGS. 4C-4D, the percent hemolysis using the 4 and 25 ejector systems were 17.58±3.56% and 18.08±2.59%, respectively. Compared to the values obtained using a single ejector (12.07±4.92% under the same operation conditions, as shown in Table 3), the use of a multi-ejector system resulted in only a moderate increase to percent hemolysis. The Kruskal-Wallis (non-parametric) analysis of variance on the experimental results indicated that the number of ejectors did not have a significant effect (p>0.05) on the percent hemolysis. Furthermore, pair-wise comparisons with nonparametric Mann-Whitney U test on hemolysis values for single to 4, 4 to 25 and single to 25 ejectors also pointed that the effect of number of ejectors is insignificant with p scores of 0.08, 1.00, and 0.08, respectively (Tables 4 and 5). These results indicated the scalability of the system to process blood at high throughput. Further, for the system to become a clinically useable method, the sterility and avoidance from microbiological contamination are very important in blood transfusion. As a first step taken towards this direction, the ejector system is operated in a sterile hood during ejection, droplet collection and thawing steps.

In summary, described in this Example herein is a scalable vitrification method for the cryopreservation of RBCs by generating nanodroplets that are vitrified and thawed at low CPA levels. The RBC cryopreservation approach presented here has potential to improve the efficiency with which global blood inventories are managed leading to significant economic and social downstream impact.

Materials and Methods

The RBC cryopreservation process consists of four main steps: blood preparation, CPA loading (FIG. 2A), ejection, and freezing/thawing/collection (FIG. 2B). The droplets were generated from the co-flow stream of the CPA-loaded RBC solution and nitrogen gas flow through an ejector (FIG. 2C). All experiments were performed in a sterile hood to prevent microbacterial contamination that could have adverse effects on the RBCs. All the abbreviations are listed in Table 7.

Blood Preparation.

All buffy coat samples were received from the Massachusetts General Hospital transfusion center. The buffy coat was easier to obtain compared to whole blood, and prepared specifically for research use. To isolate RBCs, 25 ml of buffy coat sample was first mixed with 3.5 ml of Citrate Phosphate Dextrose Adenine (CPDA-1) anticoagulant for 2 min at 20° C., and then centrifuged at 2000 rpm for 10 min at 20° C. (Allegra 6 Centrifuge, Beckman Coulter, USA). After removing the supernatant, approximately 5 ml of RBC pellets remained at the bottom. For each 100 ml of CPDA-1, 327 mg citric acid, 2.635 g sodium citrate, 222 mg monobasic sodium phosphate, 3.175 g dextrose, 27 mg adenine were used.

CPA Loading.

The CPA solutions at different concentrations (1M, 2M, 2.5M and 4M) were prepared, and the compositions were listed in Table 1. 5 ml of collected RBC pellet was first mixed with 2M glycerol at a ratio of 1:1 (v/v) (Sigma, USA) to achieve a final glycerol concentration of 1M. The mixture was then centrifuged at 2000 rpm for 10 minutes and the isolated RBCs containing 1M glycerol were collected, and then further mixed with 4M glycerol at a ratio of 1:1 (v/v) to obtain a final glycerol concentration of 2.5M.

Cell Encapsulating Droplet Generation System.

The droplet generation system is shown in FIG. 1A and FIG. 2C. An ejector was built using a 200 μl pipette tip attached to a 27 gage stainless needle tip (BD Biosciences, San Jose, Calif.). The needle tip was placed into the center of the pipette tip to build a co-flow nozzle. The two components were assembled by inserting the needle tip across pipette tip wall at a point 2 cm away from the pipette tip end. The needle tip was pushed further inside the pipette tip until it stuck out 2 mm from the edge. A nitrogen gas tank was connected to the pipette tip with TYGON® tubing (ID=3.2 mm) (Saint-Gobain Performance Plastics, Worcester, Mass.) through which the gas could flow to the pipette tip (FIG. 2C). A CPA loaded RBC sample was loaded into a syringe attached to a 30 gage needle (Small Parts Inc., Miramar, Fla.). A 15 cm polyethylene tubing (Becton Dickinson Primary Care Diagnostics, Sparks, Md.) through which the sample could flow was then used to connect the blood loaded syringe and ejector through the needle tip. The RBC sample loaded with CPAs was then loaded into a syringe pump (World Precision Instrument, Sarasota, Fla.) Nitrogen gas was flowed through the TYGON tubing to the pipette tip simultaneously while the CPA loaded RBC solution was pumped from the syringe pump and flowed through the polyethylene tubing to the needle tip, resulting in a force that created droplets from the needle tip of the ejector (FIG. 1A). The ejector was cleaned with ethanol and autoclaved before use.

Vitrification of RBCs.

The RBC sample loaded with CPAs was delivered to the ejector from the syringe pump at a flow rate of 0.2 ml/min. The nitrogen gas was supplied from the nitrogen gas tank and delivered to the ejector through TYGON® tubing at a rate of 4 l/min. For the vitrification of RBCs, the droplets were first ejected onto a polyethylene (PE) collection film (Avery, Brea, Calif.) of 8.5 mm diameter. Then, the film with the RBC droplets attached was rapidly immersed into liquid nitrogen using pre-cooled tweezers.

Thawing and Collection of RBCs.

For the thawing, the film with the CPA loaded RBC droplets attached was removed from the liquid nitrogen using tweezers and immediately transferred in a petri dish filled with a thawing solution consisting of 10 ml of 2.5M glycerol pre-warmed to 25° C. (FIG. 2C). The film was surrounded by liquid nitrogen vapor during the transfer to prevent any devitrification prior to immersion in the thawing solution. The film with the CPA loaded RBC droplets was immersed completely in the thawing solution and the droplets were washed completely off the film and into the thawing solution. Once the vitrified droplets were completely thawed and the RBCs were washed into the thawing solution, the mixture containing RBCs and 2.5M glycerol was then collected and centrifuged at 2000 RPM for 10 min. The supernatant was then removed and RBCs in 2.5M glycerol solution were collected.

Droplet Size Measurement.

Since the degree of crystallization of the droplet increases with droplet size, we measured the droplet size distribution at 5 different experimental conditions. Three major variables were investigated for their effects on droplet size distribution, including (i) droplet collection distance between ejector tip and droplet collection film, (ii) flow rate of nitrogen gas, and (iii) the flow rate of CPA loaded RBCs. At each operation condition, droplets were ejected onto the surface of a 150 mm polystyrene petri-dish, and then images were taken using a microscope (TE 2000; Nikon, Japan). The images were taken at 10× magnification, and the number of droplets and their diameters were measured using Image J (NIH, Bethesda). To create an accurate read-out of the droplet diameter, the images were first adjusted to desired threshold level (0˜255 for 8 bit gray scale image). Droplets that did not initially appear as closed circles on the image were modified using black/white threshold imaging process and the dot tools of image J. Then the data acquired was transferred to Excel where measurements were converted from pixels to micrometers to obtain droplet size (one pixel=1.28 μm). Finally, the distribution of droplet size was plotted using Sigmaplot (Systat Software Inc., Chicago, Ill.). The results were shown in (FIGS. 3A-3B).

Percent Hemolysis Analysis.

To better understand how each procedure impacts the final RBC hemolysis after vitrification, the percent hemolysis was analyzed for each step (i.e., CPA loading, droplet ejection, collecting droplets on film, and freezing/thawing). The percent hemolysis was calculated by comparing the free hemoglobin in solution after each process is performed to those of controls. The controls were the free hemoglobin present in a sample which is prepared before a given step is performed and in a sample which is prepared with DI water for 100% hemolysis value. The first control sample shows how much hemoglobin is already present before a step is performed and the second control shows how much hemoglobin is present when 100% hemolysis occurs. Percent hemolysis can be expressed by the following equation (see refs[1]-[5]):

$$Hemolysis ( \% ) = \frac{AB {S}_{process} - AB {S}_{0}}{AB {S}_{100} - AB {S}_{0}} \times 100 \qquad ( 1 )$$

Where ABS₀ and ABS_{process} are the absorbance of free hemoglobin in RBC samples before and after each step of the RBC encapsulating droplet cryopreservation process is performed, respectively. ABS₁₀₀ is the absorbance of free hemoglobin in RBC samples after total cell lysis in DI water. The numerator represents the amount of hemoglobin released during a given experimental step while the denominator represents the total amount of hemoglobin present in the RBCs before this step was performed. The fraction of hemoglobin that was intact in the cells is given by, ABS₁₀₀-ABS₀, before a given step is performed. The hemoglobin released from the cells while the step was being performed is given by, ABS_{process}-ABS₀. Dividing the numerator by the denominator gives the percentage of hemolysis. Absorbance was measured using an UV-VIS spectrophotometer (UV-2450, SHIMADZU, Japan) as shown in FIGS. 5A-5D. Hemolysis was determined using the Cripps and Harboe methods [25], which are two standard methods to calculate hemolysis based on hemoglobin absorbance at different wavelengths. Results of each process step were presented with these spectrophotometer values, Table 2.

Hemolysis Due to Loading of CPA₁ (Addition of 2M CPA to RBC Pellet for a Final 1M CPA Concentration).

To prevent ice crystals from forming in the RBCs during freezing/thawing, CPAs must be loaded to the cells. Before the CPA₁ loading step was performed, two control samples were prepared to obtain absorbance measurements (ABS₀_{_{—}}_{CPA1} and ABS₁₀_{_{—}}_{cPA1}). A 20 μl sample of isolated RBCs was added to 10 ml of Dulbecco's Phosphate Buffered Saline (DPBS) (GIBCO, Grand Island, N.Y.) to dilute the RBCs to a concentration within the range of the spectrophotometer reading where free hemoglobin is linearly correlated to the absorbance, for ABS₀_{_{—}}_{CPA1} measurement. Since minimal hemolysis occurs during the addition of DPBS, the absorbance value obtained accurately reflects the free hemoglobin concentration before 2M glycerol was loaded. To obtain a RBC sample for ABS₁₀₀_{_{—}}_{CPA1} measurement, a 20 μl sample of isolated RBCs was added to 10 ml of deionized (DI) water to obtain a total cell lysis. To perform the first CPA loading step, 1 ml of isolated RBCs was mixed with 2M glycerol with a ratio of 1:1 (v/v) (Sigma, USA) to obtain a RBC sample with a final glycerol concentration of 1M. Then 40 μl of this CPA loaded sample was mixed with 9.98 ml of 1M glycerol to match the same mixing ratios as the control samples. These three samples were then centrifuged at 2000 rpm for 10 min and the absorbance of the supernatants was measured using the UV-VIS spectrophotometer (UV-2450, Shimadzu, Japan).

Hemolysis Due to Loading of CPA₂ (Addition of 4M CPA to RBCs in 1M CPA Solution for a Final 2.5M CPA Concentration).

Two 20 μL samples of isolated RBCs loaded with 1M glycerol were taken into two tubes. One sample was diluted with 10 ml of 1M glycerol, and the other was diluted with 10 ml of DI water to obtain control samples for). For pairwise comparisons, one-tailed p-value was used to evaluate the effect of ejection distance and gas flow on percent hemolysis; whereas two-tailed p-value was used to evaluate the effect of number of ejectors on percent hemolysis.

### REFERENCES

1. Fuller B, Lane N, Benson E E, editors. Life in the Frozen State. CRC Press; 2004.
2. Standards for blood banks and transfusion services. QRB Qual Rev Bull. 1977; 3:17,22.
3. Hess J R, Greenwalt T G. Storage of red blood cells: new approaches. Transfus Med Rev. 2002; 16:283-295.
4. Hogman C F. Preparation and preservation of red cells. Vox Sang. 1998; 74(Suppl 2):177-187.
5. Scott K L, Lecak J, Acker J P. Biopreservation of red blood cells: past, present, and future. Transfus Med Rev. 2005; 19:127-142.
6. Whitaker B I, Henry R A. The 2007 Nationwide Blood Collection and Utilization Survey Report. 2007. Department of Health and Human Services, USA.
7. Timmins N E, Nielsen L K. Blood cell manufacture: current methods and future challenges. Trends Biotechnol. 2009; 27:415-422.
8. Meryman H T. Modified model for the mechanism of freezing injury in erythrocytes. Nature. 1968; 218:333-336.
9. Meryman H T, Homblower M. A method for freezing and washing red blood cells using a high glycerol concentration. Transfusion. 1972; 12:145-156.
10. M. Tullis J L, Ketchel M M, Pyle H M, Pennell R B, Gibson J G, et al. Studies on the in vivo survival of glycerolized and frozen human red blood cells. Journal of the American Medical Association. 195 8; 168:5.
11. Rowe A W, Eyster E, Kellner A. Liquid nitrogen preservation of red blood cells for transfusion; a low glycerol-rapid freeze procedure. Cryobiology. 1968; 5:119-128.
12. Pert J H, Schork P K, Moore R. Low Temperature preservation of human erythrocytes. Biochemical and clinical aspects. Bibliotheca haematologica. 1964; 19:7.
13. Krijnen H W, Wit J J F M D, Kuivenhoven A C J, Loos J A, Prins H K. Glycerol treated human red cells frozen with liquid nitrogen. Vox Sang. 1964; 9:13.
14. Wolstenholme G E W, O'Connor M, editors. The Frozen Cell. London Churchill; 1970.
15. Zadeoppe A M M. Posthypertonic hemolysis in sodium chlorid systems. Acta Physiologica Scandinavica. 1968; 73:341-&.
16. Pegg D E, Diaper M P. The effect of initial tonicity on freeze/thaw injury to human red cells suspended in solutions of sodium chloride. Cryobiology. 1991; 28:18-35.
17. Pegg D E, Diaper M P. On the mechanism of injury to slowly frozen erythrocytes. Biophysical Journal. 1988; 54:471-488.
18. Lovelock J E. The denaturation of lipid-protein complexes as a cause of damage by freezing. Proceedings of the Royal Society of London Series B-Biological Sciences. 1957; 147:427-433.
19. Mazur P, Leibo S P, Chu E H. A two-factor hypothesis of freezing injury. Evidence from Chinese hamster tissue-culture cells. Exp Cell Res. 1972; 71:345-355.
20. Fahy G M, MacFarlane D R, Angell C A, Meryman H T. Vitrification as an approach to cryopreservation. Cryobiology. 1984; 21:407-426.
21. Luyet B J, Gehenio P M. The mechanism of injury and death by low temperature. Biodynamica. 1940; 3:67.
22. Bernardin J D, Mudawar I. A Leidenfrost point model for impinging droplets and sprays. Journal of Heat Transfer-Transactions of the Asme. 2004; 126:272-278.
23. Xie H, Zhou Z W. A model for droplet evaporation near Leidenfrost point. International Journal of Heat and Mass Transfer. 2007; 50:5328-5333.
24. Song Y S, Adler D, Xu F, Kayaalp E, Nureddin A, et al. Vitrification and levitation of a liquid droplet on liquid nitrogen. Proc Natl Acad Sci USA. 2010; 107:4596-4600.
25. Malinauskas R A. Plasma hemoglobin measurement techniques for the in vitro evaluation of blood damage caused by medical devices. Artif Organs. 1997; 21:1255-1267.

### Example 2

### Nanoliter Droplet Vitrification for Oocyte Cryopreservation

Oocyte cryopreservation remains largely experimental, with live birth rates of only 2-4% per thawed oocyte. Described herein is a nanoliter droplet technology for oocyte vitrification. An ejector-based droplet vitrification system was designed to continuously cryopreserve oocytes in nanoliter droplets. Oocyte survival rates, morphologies and parthenogenetic development after each vitrification step were assessed in comparison with fresh oocytes. Oocytes were retrieved after cryoprotectant agent loading/unloading, and nanoliter droplet encapsulation showed comparable survival rates to fresh oocytes after 24 h in culture. Also, oocytes recovered after vitrification/thawing showed similar morphologies to those of fresh oocytes. Additionally, the rate of oocyte parthenogenetic activation after nanoliter droplet encapsulation was comparable with that observed for fresh oocytes. This nanoliter droplet technology enables the vitrification of oocytes at higher cooling and warming rates using lower cryoprotectant agent levels (i.e., 1.4 M ethylene glycol, 1.1 M dimethyl sulfoxide and 1 M sucrose), thus making it a potential technology to improve oocyte cryopreservation outcomes.

Oocyte cryopreservation has potential to impact preservation of female fertility by facilitating oocyte banking_{[1]}. The ability to successfully freeze oocytes would offer an alternative option to embryo cryopreservation, which is particularly important for: women who have no male partner and who wish to bank oocytes for later use; female cancer patients of reproductive age who risk losing ovarian function during extirpative surgery, chemotherapy or radiotherapy; patients who have ethical, moral or religious objections to embryo preservation_{[}2,3]

Since the first pregnancy using frozen oocytes was reported in 1986_{[4]}, several oocyte cryopreservation technologies have been developed. However, the clinical efficiency entailed in using frozen oocytes remains low, with pregnancy and live birth rates of 2-4%_{[5]}. In addition, according to the American Society of Reproductive Medicine, oocyte cryopreservation is currently considered experimental and should be performed only under an Institutional Review Board_{[6]}, which is mostly due to the low success rate of obtaining live births from thawed oocytes. Furthermore, most vitrification protocols involve manual sequential treatment of oocytes, which is laborious and requires expertise. Therefore, innovative technologies that could improve oocyte cryopreservation outcomes are urgently needed to advance the clinical practice and expand fertility preservation options for patients.

Currently, two methods exist for oocyte cryopreservation; slow freezing-rapid thawing and vitrification_{[7-9]}. The slow freezing-rapid thawing method employs relatively low levels of cryoprotectant agents (CPAs; i.e., ˜1-2 M), and it freezes oocytes at slow cooling rates typically 0.3° C./min) to prevent intracellular ice formation and minimize structural damage_{[10]}. However, oocytes cryopreserved using slow freezing are prone to hardening of the zona pellucida_{[11]}, disruption of the chromosomes_{[11,12]}, and a substantial loss in the ability to fertilize and develop in culture_{[12]}. These deleterious outcomes have been attributed to the formation of ice crystals, extreme hyperosmolarity and dehydration_{[13]}.

Due to the prevention of intracellular ice formation and the avoidance of increasing ionic concentrations of unfrozen solutions_{[14,15]}, vitrification has made it possible to increase post-thaw cell survivability compared with conventional slow freezing_{[16-18]}. Vitrification technology has been successful when employed for embryonic and somatic cells [19-22]. With vitrification, high concentrations of CPAs (i.e., ˜4-8 M) and rapid cooling rates (i.e., 1500° C./min) are required to ensure direct solidification of the vitrification solution without ice crystal formation. However, it has been suggested that high CPA levels lead to osmotic shock and toxicity to oocytes_{[23]}, thus resulting in cytoskeletal alterations_{[24-26]}, chromosome dispersal_{[27]} and spindle disassembly_{[27]}.

Various methods have been developed to reduce the overall toxicity of the CPAs that are used for vitrification, including the use of a combination of CPAs_{[28,29]} and stepwise equilibration with CPAs at room temperature_{[30]}. The most effective recent improvement in vitrification technology has been the use of minimum volume methods, including the open pulled straws technique_{[31-35]}, electron microscopy grids_{[36,37]}, the cryoloop_{[38]} and droplet-based vitrification methods_{[39-42]}. The small sample size not only enables a substantial increase in cooling rates, but also ultra high warming rates, which have been reported to be more important than cooling rates determining the survival of oocytes subjected to a vitrification procedure_{[43]}. In addition, the increase in cooling and warming rates also enables reduction in the required CPA concentrations.

Among these minimum volume methods, droplet-based vitrification offers the potential to achieve higher cooling and warming rates than carrier-based vitrification technologies. As the inventors have shown previously_{[39,44]}, this is due to the enhanced heat transfer of a droplet in the absence of a carrier, which reduces the likelihood of ice formation and the need for high CPA concentrations. However, all current droplet vitrification approaches are constrained to generate droplets manually using pipettes_{[35,42,44-47]}. Pipette methods require a high degree of technical skill and do not ensure consistency in droplet size. Furthermore, with pipette methods, it is very challenging to reliably obtain droplets smaller than 1 μl, thus limiting cooling and warming rates, and precluding the use of further reduced CPA levels. It has been demonstrated that minimizing sample volume during vitrification can potentially improve the cryopreservation outcome of oocytes by significantly increasing the cooling and warming rates and through the use of lower CPA concentrations. The technology presented in this study allows oocyte vitrification at droplets on a nanoliter scale, while currently available open procedures cannot provide reliable generation and control of droplet volumes at the nanoscale. In addition, the operator variability with the current procedures becomes more significant when handling smaller volumes. Heat transfer characteristics with these open procedures can vary as a function of the droplet size, thereby affecting the clinical results.

Recently, microscale technologies have been introduced to the cryogenics field_{[48]}, and droplet generation technologies with control over droplet sizes have been created to reliably encapsulate live functional cells_{[49-54]}. Manipulating cells encapsulated in small volumes has been presented earlier for various applications, including tissue engineering_{[44,49-51]} and diagnostics_{[55-57]}. Furthermore, cryopreservation of large volumes of blood cells on polyethylene film containing millions of droplets using a high-throughput vitrification system was also presented for storage purposes_{[53]}. The inventors have previously reported the dynamics of vitrification using a rapid nanoliter droplet encapsulation system, and demonstrated that the Leidenfrost effect is present during droplet vitrification_{[44]}. Described herein is a system and method that provides continuous generation of nanoliter droplets, by modifying the ejector-based droplet vitrification system to address limitations of the current droplet vitrification approaches. As a first step to evaluate this novel technology for oocyte cryopreservation, the mouse species was chosen as a model to optimize the protocols and the design. By vitrifying oocytes encapsulated in nanoliter droplets, this system potentially enables ultra-rapid cooling rates with reductions in required CPA concentrations. This is the first application of a scalable vitrification method in oocyte cryopreservation. Also evaluated herein is the efficiency of this vitrification system in a step-by-step fashion. At reduced CPA concentrations, oocytes survived after vitrification and warming processes. In addition, no significant effect of droplet encapsulation on oocyte survival or parthenogenetic embryo development was observed.

Hematology Analysis and Hemolysis Determination.

A hematologic analysis of the post-thaw RBCs was carried out to examine the functionality and viability of the cells with the droplet method. The number of RBCs, the hematocrit, and the total hemoglobin concentration were measured using a hematology analyzer (D3, Drew Scientific, Waterbury, Conn.). The hemolysis value (the ratio of hemoglobin concentration of cell-free supernatant to the total hemoglobin content) was obtained and the extent of RBC lysis due to cryopreservation as a function of CPA concentration was evaluated. The concentration of free hemoglobin was measured using a spectrophotometer (Shimadzu, Japan, UVProbe). For more accurate determination, the hemolysis of each RBC suspension was measured three times and averaged. The hemolysis value was weighted by the post-thaw suspension hematocrit as an indication of extracellular volume. The amount of hemolysis is calculated as:

$$Hemolysis = \frac{free hemoglobin concentration \times ( 1 - hematocrit )}{total hemoglobin concentration} \qquad Eqn . 2$$

The results showed low hemolysis value (<0.7%). These encouraging results establish the proof of concept. These values are below the accepted 1% clinical hemolysis value.

Materials & Methods

Animals & Oocyte Collection.

Female B6D2F1 mice 6-8 weeks old (Jackson Laboratory, ME, USA) were superovulated by intraperitoneal injection of 5 IU of pregnant mare serum gonadotropin (EMD Chemicals, NJ, USA) followed by the injection of 5 IU human chorionic gonadotrophin 48 h later (EMD Chemicals). A total of 15 h after human chorionic gonadotrophin treatment, mice were exposed to CO₂ until movement ceased and euthanized by cervical dislocation. A small incision was made over the midsection, the skin was reflected back and the peritoneum was entered with a sharp dissection to expose the viscera. The oviducts were immediately excised and placed into a culture dish containing 4 ml EMBRYOMAX® flushing hold medium (FHM) 4-(2-hydroxyethyl)-1-piperazineethanesulfonic acid (HEPES) buffered medium (Millipore Corporation, Billerica, M A, USA) supplemented with 4 mg/ml bovine serum albumin (BSA). Oviducts were then transferred to a center-well dish containing 1 ml FHM medium supplemented with 4 mg/ml BSA for cumulus-oocyte complex collection. While holding the oviduct in place with forceps, an incision was made in the swollen part of the oviduct using an insulin needle to allow cumulus-oocyte complex extrusion. Oocytes were then transferred to a center-well dish filled with 1 ml hyaluronidase solution (0.3 mg/ml; Sigma, Mo., USA) in FHM medium until the cumulus cells were dispersed, followed by five washes in 50 μl of FHM medium drops. Oocytes were collected in 50 μl drops of potassium simplex optimized medium (KSOM) medium (Millipore Corporation, MA, USA), covered with mineral oil (Sigma, Mo., USA) and cultured at 37° C. in 5% CO₂ and 95% air. Oocyte size measurements were obtained from 45 oocytes collected from eight mice.

Cell Encapsulating Droplet Generation System.

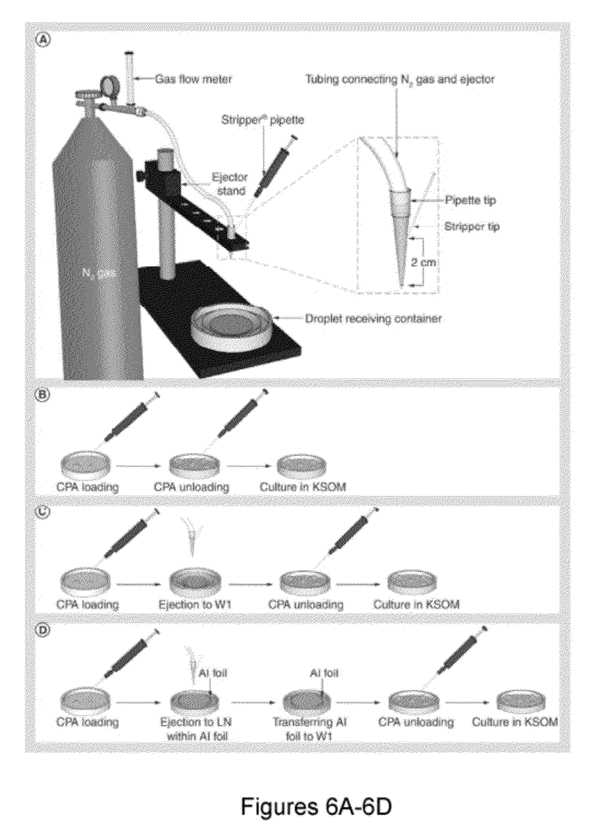

The design of the coflow droplet generation system is shown in FIG. 6A. The coflow ejector was built by placing a STRIPPER® tip (MidAtlantic Diagnostics, NJ, USA) through the small open end of a 200-μl pipette tip to create a sheath flow. The inner diameter of the STRIPPER tip was 125 μm. As shown in the magnified image of FIG. 6A, the two ejector components were assembled by inserting the STRIPPER tip across the pipette tip wall at a point 2 cm away from the small open end of the pipette tip. The STRIPPER tip was then pushed further inside the pipette tip until it protruded 2 mm from the small opening. The STRIPPER tip of the ejector was then attached to the STRIPPER pipette and the CPA loaded oocytes were aspirated into the STRIPPER tip with the aid of the STRIPPER pipette. After assembling the ejector onto the stand, TYGON® tubing (inner diameter=3.2 mm; Saint-Gobain Performance Plastics, MA, USA) attached to a nitrogen gas cylinder was connected to the ejector. Nitrogen gas was flowed through the pipette tip while CPA solution loaded with oocytes flowed through the STRIPPER tip, creating droplets from the ejector (FIG. 6A).

Vitrification & Thawing.

All the procedures were performed at room temperature (20-24° C.). In this study, mature oocytes at the M II stage, identified by extrusion of the first polar body, were used for droplet based vitrification. Before vitrification, oocytes were first suspended for 3 min in a 100-μl droplet of pre-equilibrium solution (V1) composed of 4% ethylene glycol (v/v; Sigma) and 4% dimethyl sulfoxide (v/v; Sigma) in FHM medium supplemented with 4 mg/ml BSA. Oocytes were then transferred to a 100-μl droplet of vitrification solution (V2), a mixture of 8% ethylene glycol (v/v [1.4 M]), 8% dimethyl sulfoxide (v/v [1.1 M]), and 1 M sucrose in FHM HEPES buffered medium supplemented with 4 mg/ml BSA and equilibrated for 30 s. For vitrification, oocytes in vitrification solution were first aspirated into the STRIPPER tip.

The loaded ejector was then put on the rack and the top was connected to a nitrogen gas cylinder by TYGON® tubing (FIG. 6A). Aluminum foil in a bowl shape (collection sieve) was placed in a 6-mm Petri dish and then filled with liquid nitrogen to serve as a droplet receiving container. Finally, by using the STRIPPER pipette, CPA droplets encapsulating oocytes were generated, ejected directly into liquid nitrogen and vitrified (FIG. 6A). The aluminum foil was used as a collector sieve to recover vitrified droplets for the rapid warming step.

For rapid warming, a thawing solution composed of 1 M sucrose and 4 mg/ml BSA in FHM medium (W1) was used. A culture dish containing 4 ml of W1 was prewarmed to 37° C. The aluminum foil, on which the vitrified CPA droplets encapsulating oocytes were collected, was first lifted above the liquid nitrogen surface. Immediately after liquid nitrogen evaporated, the aluminum foil was inverted and immersed into the warming solution (W1) in the dish (FIG. 6D). This procedure allowed simultaneous removal of CPAs and rapid warming. After 2.5 min, thawed oocytes recovered under stereomicroscope were sequentially transferred to graded sucrose solutions of 0.5 M (W2) and 0.2 M (W3) with 2.5 min incubation in each solution. Finally, the oocytes were transferred to FHM medium (three washes) for a total of 5 min to remove any remaining CPAs. All oocytes were then collected in 50-μl droplet of KSOM medium, covered with mineral oil and cultured in 5% CO₂/95% air at 37° C.

Droplet-Size Measurements.

The size distribution of droplets generated at different nitrogen gas flow rates (0.6, 0.7, 0.8, 0.9, 1.0 and 1.1 standard liters per min [SLPM]) was measured. At each flow condition, ejected droplets were collected on a polyethylene film (Avery, Calif., USA), and then imaged under a microscope (TE 2000; Nikon, Japan). The images were taken at 4× magnification, and the droplet sizes were measured using SPOT Imaging Solutions (Diagnostic Instruments Inc., MI, USA). Finally, the acquired data was analyzed and size distribution of droplets was plotted.

Assessment of Oocyte Morphology & Survival.

To understand how each procedure affects the ultimate oocyte survival after vitrification and thawing, oocyte morphology was analyzed 30 min and 24 h after each step (i.e., CPA loading/unloading, droplet encapsulation and vitrification/thawing). Normal oocyte morphological integrity was defined by round and regular appearance of the oocyte membrane, by refringent cytoplasm with fine homogenous granularity and by absence of signs of cytoplasmic degeneration. Fresh oocytes cultured in the KSOM medium right after retrieval were used as controls. Oocytes that appeared pycnotic or showed signs of degeneration were removed from the culture medium drop to minimize detrimental effects on the rest of the cohort.

To assess the effect of CPA loading and unloading on oocytes, CPA unloading was performed immediately following CPA loading without vitrification (FIG. 6B). Oocytes were initially suspended in V1 for 3 min and then transferred to V2 for 30 s. CPAs were then subsequently removed by transferring oocytes into W1, W2 and W3 for 2.5 min per step, followed by three washes in FHM medium for a total of 5 min. Oocytes were then cultured in KSOM medium in 5% CO₂/95% air at 37° C. for morphological analysis.

Oocyte survival was examined after nanoliter droplet encapsulation. The oocytes were first loaded with V1 and V2 as described above and then ejected into W1 in a center well culture dish (BD, NJ, USA) using the droplet generator. The ejected oocytes were then retrieved and transferred into unloading solutions (W1, W2 and W3), followed by three washes in FHM medium to completely remove CPAs. Oocytes were then cultured in KSOM medium in 5% CO₂/95% air at 37° C. for further analysis (FIG. 6C).

To further evaluate oocyte survival after vitrification and thawing, oocytes were first loaded with V1 and V2 as detailed above, and then ejected into a culture dish filled with liquid nitrogen. The droplets in the liquid nitrogen were transferred to warming solution as described in the ‘vitrification and thawing’ section. The CPAs were subsequently removed by transferring oocytes into W1, W2 and W3 followed by three washes in FHM as described above. The oocytes were finally cultured in KSOM medium in 5% CO₂/95% air at 37° C. for morphological analysis (FIG. 6D).

Parthenogenetic Activation of Oocytes.

Prior to parthenogenetic activation, oocytes were cultured in KSOM medium supplemented with 4 mg/ml BSA for 2 h to allow oocytes to recover from the previous experimental procedure. For parthenogenetic activation, oocytes were incubated in SrCl2 solution in KSOM medium at 37° C. in 5% CO₂/95% air for 2 h and then washed three-times with FHM medium. Oocytes were then transferred to KSOM medium and cultured at 37° C. in 5% CO2/95% air.

To determine the optimal SrCl2 concentration to parthenogenetically activate fresh mouse oocytes, we tested four SrCl2 concentrations (10 mM, 20 mM, 50 mM and 100 mM). Over the following 6 days, treated oocytes were examined microscopically every day for embryo development to the cleavage and blastocyst stages. Day 1 is defined as 24 h after treatment. Oocytes incubated in the regular KSOM medium without SrCl₂ were used as controls. To determine whether droplet encapsulation has effect on oocyte parthenogenetic activation, oocytes in FHM medium were ejected into a center well culture dish containing 400 μl of FHM medium. To minimize the effects of shear stress generated at the ejector tip when using medium instead of CPAs, the flow rate of nitrogen gas was reduced to 0.6 SLPM to obtain droplet sizes similar to those obtained at 0.8 SLPM using CPAs. Oocytes were retrieved and cultured in KSOM medium at 37° C. in 5% CO₂/95% air for 2 h before parthenogenetic activation. SrCl2 solution at the concentration of 50 mM, which gave the highest cleavage and blastocyst rates with control oocytes, was used for the activation of oocytes following ejection into the medium.

Statistics.

All experiments were repeated at least four times. Experimental data were analyzed statistically by nonparametric Mann—Whitney U test with pairwise comparisons (Minitab Release 14, Minitab Inc., PA, USA). The statistical significance threshold was set at 0.05 (p<0.05).

Results

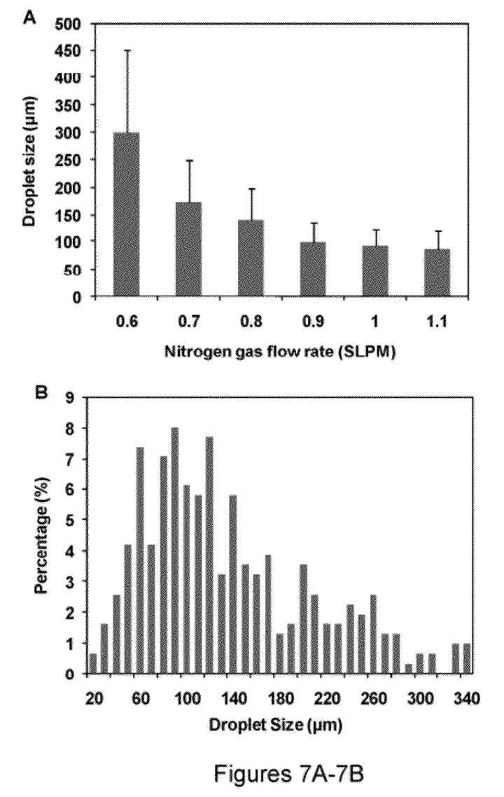

In this study, the ejection system was modified to continuously generate oocyte encapsulated nanoliter droplets, which were vitrified upon ejection into liquid nitrogen (FIG. 6A). Since the degree of crystallization during ultra-rapid cooling increases with an increase in droplet diameter_{[44]}, the ejection conditions were initially experimentally optimized to generate desirable droplet sizes for vitrification. The ideal size of droplets would allow oocytes to be vitrified at potentially the highest cooling and warming rates under the constraint of minimal volume. The minimal volume has to be large enough to satisfactorily encapsulate mouse oocytes, which are on average 92.6±4.4 μm (82-107 μm) in diameter. Droplets generated at different nitrogen flow rates (0.6-1.1 SLPM) were collected on polyethylene films and the average droplet sizes and distributions were analyzed. As shown in FIG. 7A, an increase in droplet size was observed when the nitrogen gas flow rate was reduced, and ejection at a nitrogen gas flow rate of 0.8 SLPM gave the optimal droplet sizes (140±58 μm). The size distribution of droplets generated at nitrogen gas flow rate of 0.8 SLPM is shown in FIG. 7B).

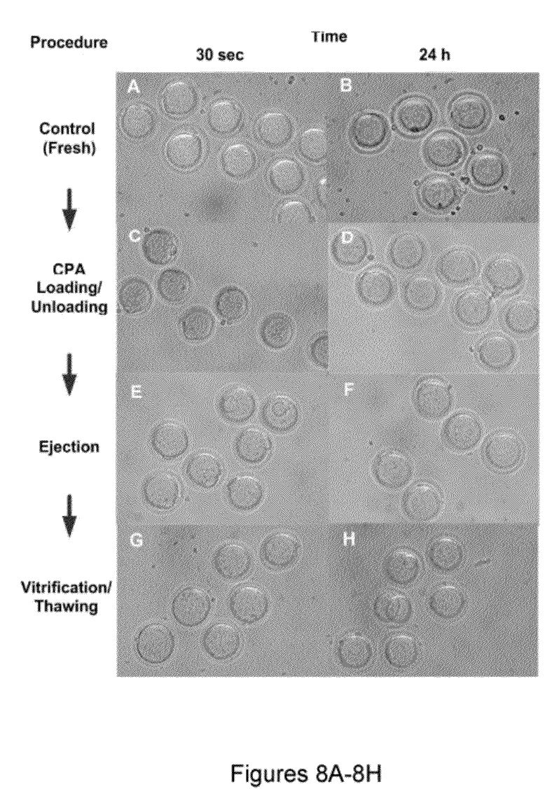

To assess the overall vitrification process using the droplet ejection system, the effect of each step (CPA loading/unloading, droplet encapsulation and vitrification/thawing) on oocyte survival rates was evaluated morphologically. As shown in FIGS. 8A-8H, oocytes survived after CPA loading and unloading, encapsulation in CPA droplets, and vitrification and thawing steps. They also showed a similar morphology as the control oocytes. There was no statistically significant difference in survival rates among the oocytes ejected in CPA droplets (89.9%), loaded and unloaded with CPAs (92.2%), and fresh control oocytes (94.9%) (Table 8). The oocyte recovery from ejection into warming medium was 83.2% (99 recovered from 119 oocytes ejected).

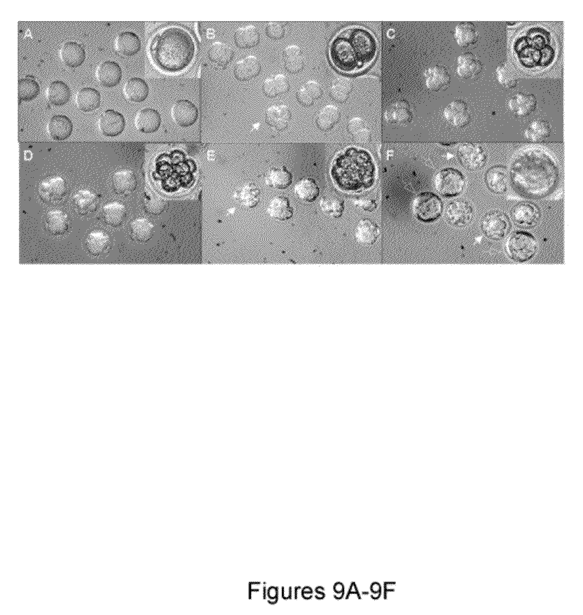

To further examine oocyte function after the oocytes were encapsulated and ejected into droplets of oocyte culture medium, parthenogenetic activation of oocytes using SrCl₂ was performed and embryo development (cleavage and blastocyst formation) compared to that observed for control oocytes. To identify the optimal SrCl₂ concentration for mouse oocyte parthenogenesis under culture conditions, fresh control oocytes were tested at different SrCl₂ concentrations (10, 20, 50 and 100 mM). 50 mM SrCl₂ resulted in the highest cleavage rate (88.9%) and blastocyst rate (49.4%) with fresh oocytes (Table 9). SrCl₂ at 50 mM was then used to parthenogenetically activate oocytes retrieved after ejection into the medium (FIG. 9A-9F). A statistically higher (p<0.01) cleavage rate (96%) was observed compared with the control group (88.6%). The blastocyst rate of ejected oocytes (26.0%) was lower (p<0.01) than that of the control group (49.4%).

Discussion

It is described herein for the first time that a simple droplet ejection system can be used to vitrify oocytes encapsulated in nanoliter CPA droplets. Oocytes cryopreserved using this system retain survivability and morphology after ejection, vitrification and thawing.

Vitrification for oocyte cryopreservation has been demonstrated to provide improved pregnancy and live birth rates compared to the conventional slow freezing method; this has been attributed to affects including the elimination of mechanical injury caused by intra- and extra-cellular ice crystal formation [8, 58]. However, as the attainable cooling and warming rates are limited due to the presence of carriers and heat transfer inefficiencies, high CPA levels are usually required during vitrification. The droplet vitrification method described here has the potential to overcome this limitation by minimizing the vitrification sample volume, and by direct contact with liquid nitrogen.

In the existing oocyte vitrification studies, droplet sizes were mostly greater than 1 μl, which limited improvement in cooling and warming rates and reduction in CPA levels. For instance, Seki and Mazur [42] showed that if the samples were warmed at a rate of 2,950° C./min, greater than 80% of the oocytes survived at the cooling rates of 187° C./min to 1,827° C./min. However, if the samples were warmed at a low rate, survival was as low as 0% regardless of the cooling rate. These results indicated the importance of achieving high warming rates for oocyte survival, which is a challenge in current technologies due to limited control over sample size. Using the ejector-based droplet generation system described here, smaller droplet sizes down to the nanoliter scale can be achieved by controlling parameters such as the rate of nitrogen gas flow at a given stripper tip size. Thus, for the application of oocyte cryopreservation, an average droplet volume of 1.4 nL was used for vitrification. This significant reduction in droplet volume allowed oocytes to be vitrified using CPA concentrations as low as 3.5 M (8% EG (v/v), 8% DMSO (v/v), and 1 M sucrose), much less than the CPA concentration used in previous studies [30, 32, 36, 59].

In addition, prior droplet vitrification studies prepared droplets manually using pipettes, which lead to inconsistent cryopreservation results due to variation in operator skills and experimental conditions. Also, these approaches are both time and labor intensive, which makes the entire vitrification process inefficient and costly. The ejection system demonstrated here enables droplet generation in a continuous manner with minimal manual involvement, thus significantly improving the vitrification efficiency and repeatability. Moreover, oocytes can be introduced into the ejector using a stripper that is commonly used in IVF clinics, making the system gentle for oocyte handling and simple for embryologists. Oocyte survival and parthenogenetic development post-ejection were evaluated to understand how the droplet generation and cell encapsulation processes affect the oocytes. While damage to the zona pellucida nor a considerable difference in oocyte survivability were not observed, a reduction in the rate of blastocyst formation was noted for ejected oocytes compared to control oocytes. The increase in cleavage rate could be partially due to mechanical stress posed on the ejector tip during ejection, which could in principle be reduced by decreasing the nitrogen air-flow rate.

Pertinent to future clinical applications, sterilization of each component of the ejection system and performing the vitrification experiments in a sterile hood are required elements of the protocol. The ejector, TYGON™ tubing and alumina foil can be sterilized using an autoclave. The nitrogen gas from the gas cylinder can be sterilized by passing through a 0.22 μm filter before reaching the ejector, while the sterilization of liquid nitrogen can be accomplished by using sterile PTFE cartridge filters [60], or by ultra-violet (UV) radiation [61]. The use of sterile liquid nitrogen should minimize potential contamination [62] during the portion of the vitrification process in which oocyte encapsulating CPA droplets come into direct contact with liquid nitrogen.

The advent of vitrification technologies was enabled by the ability to decrease the freezing volumes and increase the heat transfer rates. The recent work already presents the advancements in clinical outcomes enabled by using vitrification over slow freezing methods. The technology presented here overcomes a significant problem with vitrification technologies, which is the inability to control the freezing volume. Described herein is a system that can limit and control the droplet volumes encapsulating vitrified oocytes. This controllable process minimizes the variability in the procedures. Furthermore, the automation capabilities introduced by this technology can be useful for repeatable and reliable operations in an embryology laboratory.

Described herein is a nanoliter scale, ultra-rapid vitrification method for oocyte cryopreservation that employs continuously generated droplets vitrified at low CPA concentrations. This methodology has the potential to substantially improve the efficiency of oocyte cryopreservation and potentially the success rate of ARTs that use post-thawed oocytes.

In the light of ever-increasing survival rates in women of childbearing age with cancer, recommendation for fertility preservation at the initial treatment stage arises, as the treatment can lead to infertility. Oocyte cryopreservation presents as a viable and preferred fertility preservation option for young females and patients who have ethical or religious objections to embryo freezing. The advances in clinical cryopreservation methods have been accelerated in the recent years with the emerging nano/microscale technologies. These technologies enable manipulation of cells in nanoscale volumes reliably and repeatable. Using these technological advances to address oocyte biopreservation, which is still considered challenging and experimental, may lead to automated routine clinical procedures increasing successful biopreservation outcomes. The results described herein relate to new vitrification strategies and improved protocols. Aspects of the invention described herein can have a significant impact on the long-term storage for both clinical and research applications by combining the low CPA toxicity of slow freezing with diminished ice crystal formation of vitrification addressing the challenging demands of cryopreservation technologies. Further, this approach creates new pathways to biopreserve other biological materials including stem cells, and human tissues, e.g. blood leading to potential broad medical applications.

### REFERENCES

1. Gidoni, Y, Holzer, H, Tulandi, T, and Tan, S L: Fertility preservation in patients with non-oncological conditions. Reprod Biomed Online. 16(6), 792-800 (2008).
2. Vogelzang, N J: Treatment options in metastatic renal carcinoma: an embarrassment of riches. J Clin Oncol. 24(1), 1-3 (2006).
3. Medicine, ECotASfR: Fertility preservation and reproduction in cancer patients. Fertil Steril. 83(6), 1622-1628 (2005).
4. Chen, C: Pregnancy after human oocyte cryopreservation. Lancet. 1(8486), 884-886 (1986).
5. Oktay, K, Cil, A P, and Bang, H: Efficiency of oocyte cryopreservation: a meta-analysis. Fertil Steril. 86(1), 70-80 (2006).
6. Practice Committee of Society for Assisted Reproductive, T, and Practice Committee of American Society for Reproductive, M: Essential elements of informed consent for elective oocyte cryopreservation: a Practice Committee opinion. Fertil Steril. 90(5 Suppl), S134-135 (2008).
7. Tao, T, Zhang, W, and Del Valle, A: Human oocyte cryopreservation. Curr Opin Obstet Gynecol. 21(3), 247-252 (2009).
8. Fadini, R, Brambillasca, F, Renzini, M M, Merola, M, Comi, R, De Ponti, E, and Dal Canto, M B: Human oocyte cryopreservation: comparison between slow and ultrarapid methods. Reprod Biomed Online. 19(2), 171-180 (2009).
9. Agca, Y: Cryopreservation of oocyte and ovarian tissue. ILAR J. 41(4), 207-220 (2000).
10. Fabbri, R, Porcu, E, Marsella, T, Rocchetta, G, Venturoli, S, and Flamigni, C: Human oocyte cryopreservation: new perspectives regarding oocyte survival. Hum Reprod. 16(3), 411-416 (2001).
11. Lucena, E, Bernal, D P, Lucena, C, Rojas, A, Moran, A, and Lucena, A: Successful ongoing pregnancies after vitrification of oocytes. Fertil Steril. 85(1), 108-111 (2006).
12. Bernard, A, and Fuller, B J: Cryopreservation of human oocytes: a review of current problems and perspectives. Hum Reprod Update. 2(3), 193-207 (1996).
13. Gratwohl, A: Thomas' hematopoietic cell transplantation. Eur J Haematol. 84(1), 95 (2010).
14. Fahy, G M, MacFarlane, D R, Angell, C A, and Meryman, H T: Vitrification as an approach to cryopreservation. Cryobiology. 21(4), 407-426 (1984).
15. Rayos, A A, Takahashi, Y, Hishinuma, M, and Kanagawa, H: Quick freezing of unfertilized mouse oocytes using ethylene glycol with sucrose or trehalose. J Reprod Fertil. 100(1), 123-129 (1994).
16. Lin, T K, Su, J T, Lee, F K, Lin, Y R, and Lo, H C: Cryotop vitrification as compared to conventional slow freezing for human embryos at the cleavage stage: survival and outcomes. Taiwan J Obstet Gynecol. 49(3), 272-278 (2010).
17. Desai, N, Abdelhafez, F, Ali, M Y, Sayed, E H, Abu-Alhassan, A M, Falcone, T, and Goldfarb, J: Mouse ovarian follicle cryopreservation using vitrification or slow programmed cooling: Assessment of in vitro development, maturation, ultra-structure and meiotic spindle organization. J Obstet Gynaecol Res (2010).
18. Keskintepe, L, Agca, Y, Sher, G, Keskintepe, M, and Maassarani, G: High survival rate of metaphase II human oocytes after first polar body biopsy and vitrification: determining the effect of previtrification conditions. Fertil Steril. 92(5), 1706-1715 (2009).
19. Mukaida, T, Takahashi, K, and Kasai, M: Blastocyst cryopreservation: ultrarapid vitrification using cryoloop technique. Reprod Biomed Online. 6(2), 221-225 (2003).
20. Liebermann, J, Tucker, M J, Graham, J R, Han, T, Davis, A, and Levy, M J: Blastocyst development after vitrification of multipronuclear zygotes using the Flexipet denuding pipette. Reprod Biomed Online. 4(2), 146-150 (2002).
21. Abdelhafez, F F, Desai, N, Abou-Setta, A M, Falcone, T, and Goldfarb, J: Slow freezing, vitrification and ultra-rapid freezing of human embryos: a systematic review and meta-analysis. Reprod Biomed Online. 20(2), 209-222 (2010).
22. Magalhaes, R, Wang, X W, Gouk, S S, Lee, K H, Ten, C M, Yu, H, and Kuleshova, L L: Vitrification successfully preserves hepatocyte spheroids. Cell Transplant. 17(7), 813-828 (2008).
23. Arav, A, Shehu, D, and Mattioli, M: Osmotic and cytotoxic study of vitrification of immature bovine oocytes. J Reprod Fertil. 99(2), 353-358 (1993).
24. Vincent, C, and Johnson, M H: Cooling, cryoprotectants, and the cytoskeleton of the mammalian oocyte. Oxf Rev Reprod Biol. 14, 73-100 (1992).
25. Vincent, C, Garnier, V, Heyman, Y, and Renard, J P: Solvent effects on cytoskeletal organization and in-vivo survival after freezing of rabbit oocytes. J Reprod Fertil. 87(2), 809-820 (1989).
26. Joly, C, Bchini, 0, Boulekbache, H, Testart, J, and Maro, B: Effects of 1,2-propanediol on the cytoskeletal organization of the mouse oocyte. Hum Reprod. 7(3), 374-378 (1992).
27. Saunders, K M, and Parks, J E: Effects of cryopreservation procedures on the cytology and fertilization rate of in vitro-matured bovine oocytes. Biol Reprod. 61(1), 178-187 (1999).
28. Fahy, G M, Wowk, B, Wu, J, and Paynter, S: Improved vitrification solutions based on the predictability of vitrification solution toxicity. Cryobiology. 48(1), 22-35 (2004).
29. Eroglu, A: Cryopreservation of mammalian oocytes by using sugars: Intra- and extracellular raffinose with small amounts of dimethylsulfoxide yields high cryosurvival, fertilization, and development rates. Cryobiology. 60(3 Suppl), S54-59 (2010).
30. Wang, Z, Sun, Z, Chen, Y, and He, F: A modified cryoloop vitrification protocol in the cryopreservation of mature mouse oocytes. Zygote. 17(3), 217-224 (2009).
31. Vajta, G, Holm, P, Kuwayama, M, Booth, P J, Jacobsen, H, Greve, T, and Callesen, H: Open Pulled Straw (OPS) vitrification: a new way to reduce cryoinjuries of bovine ova and embryos. Mol Reprod Dev. 51(1), 53-58 (1998).
32. Suo, L, Zhou, G B, Meng, Q G, Yan, C L, Fan, Z Q, Zhao, X M, Fu, X W, Wang, Y P, Zhang, Q J, and Zhu, S E: OPS vitrification of mouse immature oocytes before or after meiosis: the effect on cumulus cells maintenance and subsequent development. Zygote. 17(1), 71-77 (2009).
33. Chen, S U, Lien, Y R, Chen, H F, Chao, K H, Ho, H N, and Yang, Y S: Open pulled straws for vitrification of mature mouse oocytes preserve patterns of meiotic spindles and chromosomes better than conventional straws. Hum Reprod. 15(12), 2598-2603 (2000).
34. Chen, S U, Lien, Y R, Cheng, Y Y, Chen, H F, Ho, H N, and Yang, Y S: Vitrification of mouse oocytes using closed pulled straws (CPS) achieves a high survival and preserves good patterns of meiotic spindles, compared with conventional straws, open pulled straws (OPS) and grids. Hum Reprod. 16(11), 2350-2356 (2001).
35. Martino, A, Songsasen, N, and Leibo, S P: Development into blastocysts of bovine oocytes cryopreserved by ultra-rapid cooling. Biol Reprod. 54(5), 1059-1069 (1996).
36. Kim, S H, Ku, S Y, Sung, K C, Kang, M J, Kim, S A, Kim, H S, Oh, S K, Jee, B C, Suh, C S, Choi, Y M, Kim, J G, and Moon, S Y: Simplified EM grid vitrification is a convenient and efficient method for mouse mature oocyte cryopreservation. Yonsei Med J. 47(3), 399-404 (2006).
37. Lane, M, Schoolcraft, W B, and Gardner, D K: Vitrification of mouse and human blastocysts using a novel cryoloop container-less technique. Fertil Steril. 72(6), 1073-1078 (1999).
38. Xu, F, Moon, S, Zhang, X, Shao, L, Song, Y S, and Demirci, U: Multi-scale heat and mass transfer modelling of cell and tissue cryopreservation. Philos Transact A Math Phys Eng Sci. 368 (1912), 561-583 (2010).
39. Landa, V, and Tepla, O: Cryopreservation of mouse 8-cell embryos in microdrops. Folia Biol (Praha). 36(3-4), 153-158 (1990).
40. Kuwayama, M: Highly efficient vitrification for cryopreservation of human oocytes and embryos: the Cryotop method. Theriogenology. 67(1), 73-80 (2007).
41. Papis, K, Shimizu, M, and Izaike, Y: Factors affecting the survivability of bovine oocytes vitrified in droplets. Theriogenology. 54(5), 651-658 (2000).
42. Seki, S, and Mazur, P: The dominance of warming rate over cooling rate in the survival of mouse oocytes subjected to a vitrification procedure. Cryobiology. 59(1), 75-82 (2009).
43. Song, Y S, Adler, D, Xu, F, Kayaalp, E, Nureddin, A, Anchan, R M, Maas, R L, and Demirci, U: Vitrification and levitation of a liquid droplet on liquid nitrogen. Proc Natl Acad Sci USA. 107(10), 4596-4600 (2010).
44. Bagis, H, Sagirkaya, H, Mercan, H O, and Dinnyes, A: Vitrification of pronuclear-stage mouse embryos on solid surface (SSV) versus in cryotube: comparison of the effect of equilibration time and different sugars in the vitrification solution. Mol Reprod Dev. 67(2), 186-192 (2004).
45. Dhali, A, Anchamparuthy, V M, Butler, S P, Pearson, R E, Mullarky, I K, and Gwazdauskas, F C: Gene expression and development of mouse zygotes following droplet vitrification. Theriogenology. 68(9), 1292-1298 (2007).
46. Dinnyes, A, Dai, Y, Jiang, S, and Yang, X: High developmental rates of vitrified bovine oocytes following parthenogenetic activation, in vitro fertilization, and somatic cell nuclear transfer. Biol Reprod. 63(2), 513-518 (2000).
47. Zhang, X, Catalano, P N, Gurkan, U A, Khimji, I, and Demirci, U: Emerging Technologies in Medical Applications of Minimum Volume Vitrification. Nanomedicine (Lond). In press (2011).
48. Song, Y S, Moon, S, Hulli, L, Hasan, S K, Kayaalp, E, and Demirci, U: Microfluidics for cryopreservation. Lab Chip. 9(13), 1874-1881 (2009).
49. Demirci, U, and Montesano, G: Cell encapsulating droplet vitrification. Lab Chip. 7(11), 1428-1433 (2007).
50. Demirci, U, and Montesano, G: Single cell epitaxy by acoustic picolitre droplets. Lab Chip. 7(9), 1139-1145 (2007).
51. Moon, S, Hasan, S K, Song, Y S, Xu, F, Keles, H O, Manzur, F, Mikkilineni, S, Hong, J W, Nagatomi, J, Haeggstrom, E, Khademhosseini, A, and Demirci, U: Layer by layer three-dimensional tissue epitaxy by cell-laden hydrogel droplets. Tissue Eng Part C Methods. 16(1), 157-166 (2010).
52. Xu, F, Moon, S J, Emre, A E, Turali, E S, Song, Y S, Hacking, S A, Nagatomi, J, and Demirci, U: A droplet-based building block approach for bladder smooth muscle cell (SMC) proliferation. Biofabrication. 2(1), 014105 (2010).
53. Samot, J, Moon, S, Shao, L, Zhang, X, Xu, F, Song, Y, Keles, H O, Matloff, L, Markel, J, and Demirci, U: Blood banking in living droplets. PLoS One. 6(3), e17530 (2011).
54. Moon, S, Kim, Y G, Dong, L, Lombardi, M, Haeggstrom, E, Jensen, R V, Hsiao, L L, and Demirci, U: Drop-on-demand single cell isolation and total RNA analysis. PLoS One. 6(3), e17455 (2011).
55. Kim, Y G, Moon, S, Kuritzkes, D R, and Demirci, U: Quantum dot-based HIV capture and imaging in a microfluidic channel. Biosens Bioelectron. 25(1), 253-258 (2009).
56. Moon, S, Keles, H O, Ozcan, A, Khademhosseini, A, Haeggstrom, E, Kuritzkes, D, and Demirci, U: Integrating microfluidics and lensless imaging for point-of-care testing. Biosens Bioelectron. 24(11), 3208-3214 (2009).
57. Zhang, X, Khimji, I, Gurkan, U A, Safaee, H, Catalano, P N, Keles, H O, Kayaalp, E, and Demirci, U: Lensless imaging for simultaneous microfluidic sperm monitoring and sorting. Lab Chip 11, 2535-2540 (2011).
58. Chen, S U, and Yang, Y S: Slow freezing or vitrification of oocytes: their effects on survival and meiotic spindles, and the time schedule for clinical practice. Taiwan J Obstet Gynecol. 48(1), 15-22 (2009).
59. Lane, M, and Gardner, D K: Vitrification of mouse oocytes using a nylon loop. Mol Reprod Dev. 58(3), 342-347 (2001).
60. McBurnie, L D, and Bardo, B: Validation of sterile filtration of liquid nitrogen. Pharm Technol North America. 26, 9 (2002).
61. Parmegiani, L, Cognigni, G E, and Filicori, M: Ultra-violet sterilization of liquid nitrogen prior to vitrification. Hum Reprod. 24(11), 2969 (2009).
62. Bielanski, A, and Vajta, G: Risk of contamination of germplasm during cryopreservation and cryobanking in IVF units. Hum Reprod. 24(10), 2457-2467 (2009).

### Example 3

### High Throughput, Automated, Vitrification Based Blood Cryopreservation in a Closed System

Globally, millions of health complications result from large-scale blood shortages during natural disasters and military conflicts, as well as local shortages in clinical settings due to fluctuations in supply and demand¹. Long-term cryopreservation of blood products provides an inventory to help meet the demand during such shortages by freezing excess blood. Although the use of additive preservatives has extended the liquid storage of blood products to several weeks (i.e. 42 days for red blood cells (RBCs)²⁻⁴), limited shelf life makes it difficult to manage blood inventories resulting in a large waste (˜0.2 billion USD annually)⁶. Thus, there is a significant need for new approaches to amend the way blood is stored, transported and handled in war, global disaster zones as well as local emergencies. Improved blood cryopreservation methods will prevent waste, and reduce vulnerability to shortages.

Although blood donation rate in the US has continually declined since 1987, the use of donated blood products has increased considerably²³, which calls for new and efficient blood freezing methods. In 2006, 15.7 million units of blood were collected, screened, processed, and made available in the US. Of that total amount, 14.5 million units were transfused and 1.2 million units were discarded due to short shelf-life. This represents a significant waste of the available blood supply. The shelf-life of liquid blood products (e.g., 42 days at 4° C. for RBCs and 5 days at 20° C. for platelets) requires careful inventory management, particularly for Type O (“universal donor”) and rare blood types. Frozen blood products, in contrast, can be stored longer. At −80° C., RBCs, plasma, and platelets can be stored for 10, 7, and 2 years, respectively²⁴.

For each method, controlled addition and removal of glycerol is required to prevent osmotic lysis of the RBCs. In addition, it is important to reduce the intracellular glycerol concentration to about 2% post thawing, otherwise hemolysis of the preserved RBCs will occur after transfusion²⁶. Both RBC cryopreservation methods can provide 75% in vivo survival 24 hours post transfusion with sufficient glycerol removal²⁷.

The conventional vitrification methods suffer from low-throughput and poor heat transfer. Blood biopreservation needs to achieve high volume processing to provide clinical relevance and practical use. In addition, the present cryopreservation approaches require tedious manual work in handling cells between CPA loading and unloading solutions⁵⁹. In general, the current blood freezing methods utilize an open system where freezing and thawing of RBCs occur. As a result, the post-thaw shelf-life of blood products is 24 hours, which enforces a very tight schedule for use^{6, 29}. On the other hand, it has been reported that closed systems for blood biopreservation can extend the shelf-life after thawing up to 14 days by preventing any potential bacterial contaminations⁶. Therefore, the potential high impact of closed systems in blood cryopreservation is apparent and warrants further research and development efforts.

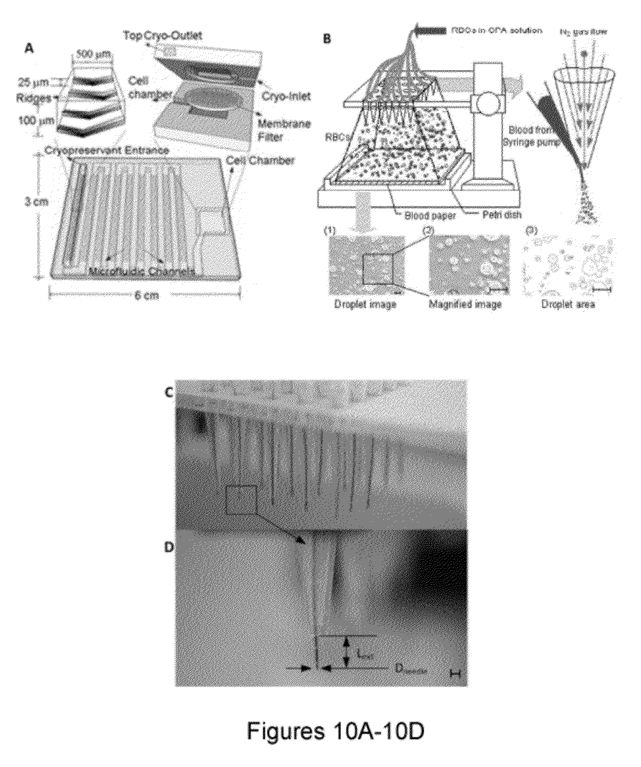

As an alternative cryopreservation method, vitrification has provided a means to significantly reduce the damage to various cells and tissues^{18, 19}. Vitrification offers advantages since ice crystal formation and the corresponding intra and extracellular solute accumulation are prevented. However, broad application of RBC vitrification in clinics hasn't yet been achieved. Challenges include (i) toxicity and osmotic shock of high CPA concentrations needed to vitrify suspensions at a realizable temperature, (ii) difficulty to achieve extreme cooling and thawing rates, (iii) devitrification upon rewarming, and (iv) inability to accommodate freezing of large volumes of blood using microliter cryovials. Herein is described a new platform (FIG. 10A-10D) to address these challenges.

The blood biopreservation is a 12 billion USD market²⁰, which impacts millions of lives. The systems and methods described herein achieve vitrification using significantly lower CPA levels than existing vitrification methods (four fold lesser), thus leading to high cell survivability, reduced osmotic effects, and minimum manual intervention²¹. Multiple nozzles ejecting millions of droplets per second can enable high-throughput operation (e.g. less than 10 minutes per blood unit with an array of hundred ejectors). This method also provides a new regime of heat transfer to vitrify cells, thereby making vitrification clinically practical and attainable for blood biopreservation. The clinical advantages of vitrification over slow freezing are reported for RBCs⁶ and for oocytes²². Described herein in this Example are systems and methods for the use of vitrification in clinical practice for blood cryopreservation. The result of the aspects of the invention described herein are ultra-rapid cooling rates (10,000° C./sec) and low levels of ice formation, thus leading to improved functionality and longer shelf life. There is a significant clinical need to improve the viability, stability, affordability, and availability of whole blood and blood components needed during peace, war, and disasters. The proposed technology can potentially transform the operational logistics of national and global blood supply. This platform has broad range of applicability to other fields and cell types including oocytes and stem cells.

Described herein is an automatic microchip freezing platform that will offer two major technological advantages: 1) reducing osmotic and mechanical stress on cells through progressive loading/unloading of CPAs with minimal handling, and 2) high-throughput vitrification with droplet-based method at ultra rapid cooling rates at low CPA concentrations. Described herein is an automatic disposable microchip that exposes RBCs to CPAs in a controlled and progressive manner, minimizing physical osmotic stress to the cells, and reducing manual handling. Described herein is a method for achieving high-throughput vitrification of RBCs by encapsulating in nanodroplets allowing high heat transfer rates and using minimum CPA concentrations. The methods and systems described herein in this Example can eliminate manual cell handling and significantly reduce damage due to osmotic imbalance and physical forces. Furthermore, the methods and systems described can be automated and can be designed as a closed system. The integrated method that is described herein is a pioneer, which minimizes the risk of bacterial contamination and readily prepares RBCs for long term frozen and extended post-thaw storage.

The aspects of the invention described herein provide a technological solution to the current challenges in blood cryopreservation. The aspects of the invention described herein specifically address the following: 1) avoid IIF due to ultra-fast cooling, 2) achieve high processing rates enabling to handle large blood volumes, 3) use very low CPA concentrations to minimize cytotoxicity, 4) provides continuous addition and removal of CPAs to minimize osmotic shock, and 5) minimize evaporation and fluid shear stress to cells. The methods and systems described herein solve the acute needs of vitrification of RBCs at high throughput and can improve cell survival rates and function. Described herein is a microfluidic system for CPA loading/unloading (FIG. 10A), which minimizes both osmotic shock and mechanical stress to cells³⁶. This is a broadly enabling platform, applicable to practically all cell types, especially therapeutic RBCs, peripheral blood stem cells, and primary hepatocytes.

Rapid Vitrification with Cells Encapsulated in Droplets

Three factors affect vitrification: cooling rate, CPA viscosity, and droplet volume³⁷⁻³⁹. Increasing the cooling rate enhances vitrification⁴⁰. This can be achieved by either increasing the CPA viscosity (molarity) or decreasing the droplet size. Vitrification at low CPA concentrations is preferred since it reduces cytotoxicity. The rate of heat transfer per unit area in an object is proportional to the temperature gradient across the boundary (Fourier's law of cooling). The inventors have previously shown that a system for droplet-based vitrification can achieve extremely fast cooling rates of around 10,000° C./min, which is 100 times faster than conventional vitrification method⁵. Consequently, this can minimize possible intracellular ice crystallization during freezing and thawing. The inventors' prior work demonstrated an understanding of the behavior of droplets in liquid nitrogen, and a model to predict a cut-off diameter for vitrification of droplets⁵.

Protocol Optimization as Function of Drop Size

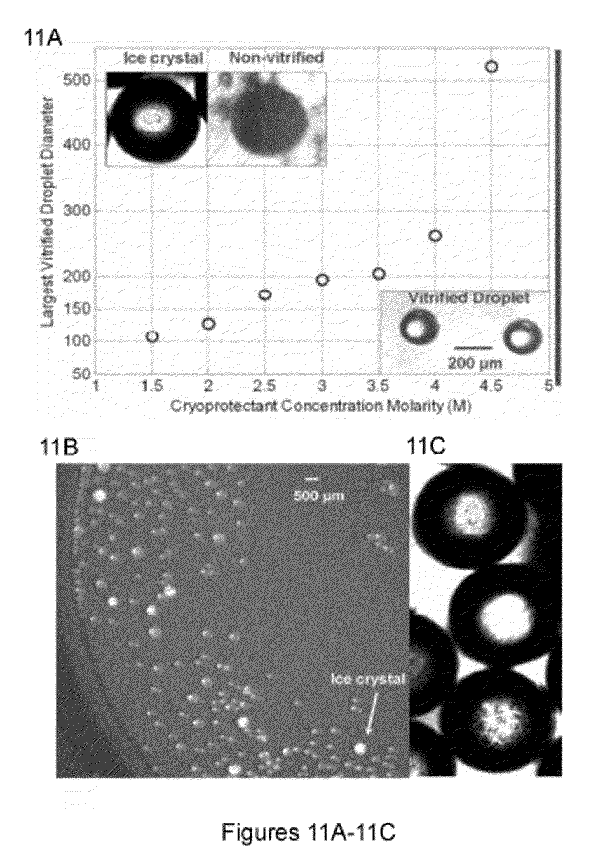

The experimental setup was as described in Song et al. PNAS 2010. The correlation between vitrified droplet size and CPA concentration was characterized (FIGS. 11A-11C). Circular points in the graph indicate the largest droplets that were vitrified (FIG. 11A). As predicted theoretically, smaller droplets vitrified at lower CPA concentration than larger droplets. The inserted image (bottom right) shows vitrified droplets (194 μm, 3 M PrOH) whereas the inserted image (top left) shows non-vitrified droplets (525 μm, 4.5 M PrOH), indicating the possibility to vitrify droplets encapsulating cells at low CPA level by using small droplets. FIG. 11B shows droplets of various sizes after ejection into liquid nitrogen with a high magnification image (60×) shown in FIG. 11C.

Cell Viability Before and after Vitrification

To ensure that the droplet ejector does not adversely affect cell viability, AML-12 hepatocytes were initially ejected with ejection rates between 1 and 1000 droplets per second. The sizes of AML-12 cells were 18-23 μm in diameter, whereas those of droplets were around 70 μm. The cell viability exceeded 90% as per standard live/dead assays, which only decreased by 2-7% with respect to the controls, indicating that the ejector does not harm the cells. Furthermore, when the cells were cultured immediately after ejection, they adhered to the culture dish, and showed comparable morphology to those in initial culture and reached confluence in a week. Their metabolic activity was also demonstrated by using a Vybrant Cell Metabolic Assay Kit (V-23110, Invitrogen, MA).

Minimizing Osmotic Shock in Microfluidic Channels

Microfluidic Device Modeling, Design and Fabrication.

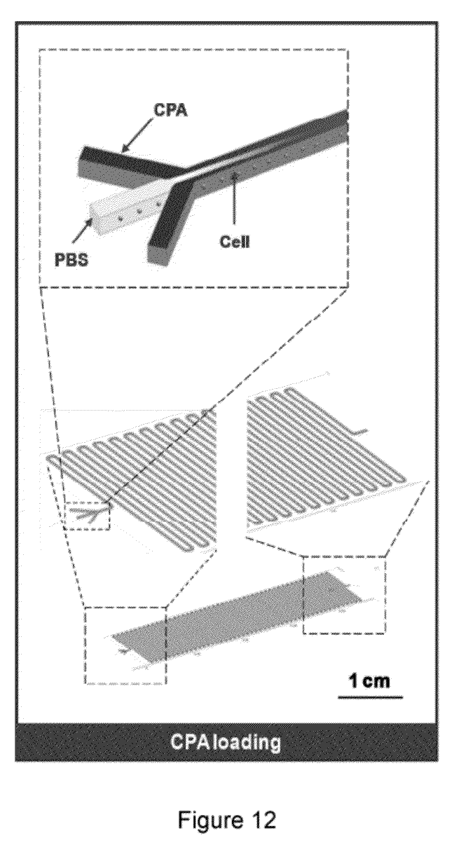

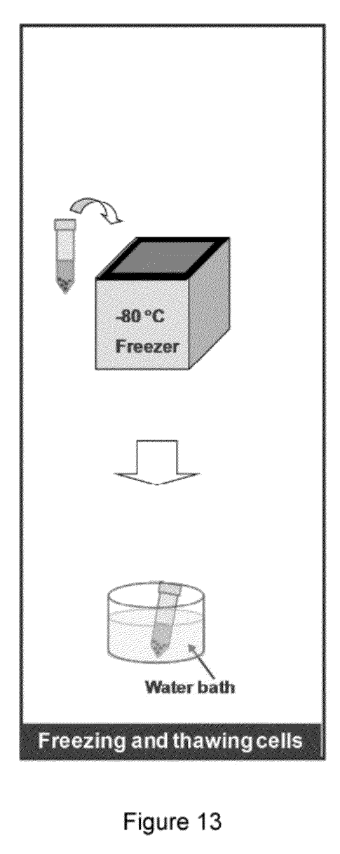

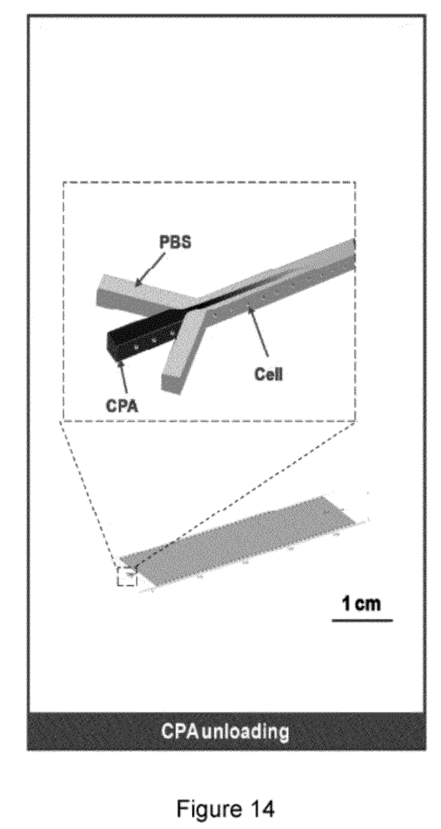

Described herein is a microfluidic device (FIG. 12) to aid cryopreservation (FIG. 13) that provides the ability to increase and decrease CPA concentrations in a controlled, progressive manner without changing CPA exposure time (<10 minutes). The microfluidic channel has two inputs: one for cells and another for CPAs. As shown in FIG. 12, cells are injected into the middle channel and CPAs into the side channels. Cells experience CPA concentration changes progressively, thus minimizing osmotic shock. By regulating the flow rates of CPAs and cell suspension, we can actively control the CPA concentrations and exposure time. After the freezing and thawing steps, the same microfluidic concept was used for CPA unloading by using PBS solution (FIG. 14).

Cell Viability and Functionality after Progressive Loading/Unloading of CPAs.

Based on mass transport modeling through a cell membrane, appropriate microchannel dimensions (100×100 μm×1.5 m) were determined using a model cell line (HepG2, hepatocytes). CPA loading and unloading durations were 0.75 min and 7.5 min, respectively. The cell viabilities at different stages (initial flask, before freezing and after thawing) were characterized using live/dead assays. The cells were transferred in a cryotube and stored in a freezer (−80° C.) and thawed after one day in a warm water bath (37° C.) and flowed into the microchannels to unload CPAs. Also, culture of post-thawed cells was observed for proliferation for 7 days. Compared to the control samples prepared using one-step or stepwise (2M and 3M) loading/unloading protocols, higher cell viabilities were demonstrated with microfluidic approach compared to the one-step protocol. In summary, the microfluidic approach resulted in ˜25% and ˜10% higher viability than the one-step and stepwise approaches, respectively, due to reduction of osmotic shock.

Design and Fabrication of Microfluidic Device for CPA Loading/Unloading.

Described herein is an automatic disposable microchip that exposes RBCs to CPAs in a controlled and progressive manner, minimizing physical osmotic stress to the cells, and reducing manual handling. This microfluidic based CPA loading/unloading platform effectively exposes the RBCs to a smooth CPA gradient. This achieves improved RBC viability and functionality due to decreased toxicity and osmotic shock caused by sudden CPA exposure in the current methods.

The CPA and cell suspension are initially fed into the microfluidic device with a control at μl/sec resolution. Channel length, flow rate, and ridge structure spacing are optimized for rapid, efficient, fluid mixing, a process that occurs in approximately 30 seconds using mathematical model. Based on the results, devices can be fabricated with varying channel lengths ranging from 5 cm to 60 cm to provide sufficient residence time for cells in the device at various flow rates. The microchannels will be 500 μm wide and 100 μM high and operated at flow rates up to 10 ml/hr. Herringbone structures, 25 μm wide and 20 μm deep will be placed 25 μm apart throughout the microfluidic channel. The devices can be fabricated using micromachining techniques.

The cells can flow along the microfluidic channel with CPAs and be protected from shear since the flow locates them in the center of the channel. To optimize the design and to monitor diffusion throughout the channel, the CPA concentration profile can be verified throughout the device. Fluorescent dies and beads can be used for initial optimization with fluorescence intensity measured. The flow rates of cells passing through the channel can be <10-20 ml/hr, laminar flow. RBCs exit at the channel outlet once the desired CPA exposure protocol is completed. The microchip outlet can be interfaced to the inlet of the droplet ejector. Alternatively, the cells that exit the microfluidic channel can be collected into a Falcon tube, and then loaded into the droplet generator device for droplet-based vitrification. Interfacing the microchip with droplet-generator reservoir can be accomplished by linking multiple microfluidic devices with tubing.

Optimizing the Flow Rate and Timing for Optimal Cryoprotectant Exposure.

Cells can be flowed through the microfluidic channel along with the CPA. By changing the flow rate, chamber size, and CPA concentration that flows through the microfluidic channels, it is possible to control how quickly and for how long the cells are exposed to CPA. A long device can achieve smooth diffusion profiles also at high flow rates. At the same time, the devices can be small, short and simple. Device dimensions and flow rates can be optimized to smoothly expose cells (e.g. RBCs) to different CPA concentration levels (0.5-6M). The use of microfluidic pumps allows control of the flow rates of RBCs (10±1 μl/min) and cryoproteciants (<10±1 μl/min) entering the channel with great precision. Further, the devices will be fabricated using microfabrication methods, which allow submicron precision in device dimensions (<1 μm) using standard lithographical methods. This precise control of flow rate and device dimensions permits accurate control of the diffusion process.

Throughput Analysis of Multiple Ejector System.

Described herein is the high-throughput vitrification of cells (e.g. RBCs) by encapsulating in nanodroplets allowing high heat transfer rates and using minimum CPA concentrations. The overall system is designed to be high throughput in terms of blood volume that it can process. An easy-to-use, closed, disposable nanodroplet ejector cartridge as described herein can freeze one unit (450 ml) of RBCs. The 25-ejector test model described herein in Example 1 has shown that an output rate of 20 ml/min can be attained. Thus, the device described herein in this Example will need less than 10 minutes to freeze RBCs present in one unit of blood, which is faster than current blood freezing methods⁴¹.

As shown in Example 1 herein, 25 ejectors can be simply assembled onto a PMMA frame patterned with holes in 5×5 array (total size ejector is 4 mm in diameter) within an 8×10 cm² area. For a system using N ejectors (e.g. N=100), 25 ejectors can be packed as one unit and then connected through tubing to a single CPA-loaded RBC solution and N₂ gas tank with flow rates increased by N times. Since only one RBC solution and one N₂ gas tank will be used for the multiple ejector system, an increase in throughput does not increase the complexity of the system. The challenge with a multiple system would be to achieve even flow rates distributed to each ejector. This variation can be <0.1% and not lead to significant difference in droplet sizes.

To achieve fast thawing of RBCs post freezing, an efficient heat exchange system using a large surface area approach, referred to herein as “blood paper” was described in Example 1 herein. Blood paper is a thin sheet of polyethylene pe material which provides a large surface area to store and retrieve blood easily. By using the blood paper, droplets can be handled in more automated and high-throughput manner, especially for storage and thawing steps. Low hemolysis values (<0.7%) have been shown in the results described herein when “blood paper” was used. Furthermore, the inventors have verified that collecting droplets on a surface with a wide area is an effective way to avoid accumulation of the vitrified droplets, which would lower the heat exchange rates during thawing step, and could even adversely affect the overall outcome of the blood biopreservation. The blood paper, which is easy to store as stacks for future applications and capability to hold large volumes is an approach that facilitates the handling of millions of vitrified droplets with minimal hemolysis. The rapid heat release during thawing is as important as the freezing step to achieve low hemolytic values. The new system described herein is an automated, functionally closed system for the glycerolization and deglycerolization of RBCs, which minimizes the risk of bacterial contamination and readily prepares RBCs for long-term storage and extended post-thaw shelf-life. Extended post-thaw storage would provide more time for quarantine and donor retesting before transfusion. The automated glycerolization/deglycerolization processes significantly reduce labor and processing time while providing more efficient RBCs management.

Thawing Cell and Analyzing Survival and Viability.

Survival of frozen-thawed cells can be compared to those cryopreserved using conventional vitrification methods and fresh controls. The function of post-thaw cells (e.g. RBCs) can also be assessed. The cells will be vitrified with an optimized protocol, and the “blood paper” with vitrified cells will be rapidly thawed with warmed 2 M glycerol solution. The container will be kept on an orbital shaker for about 10 min to allow the cells to completely detach from the “blood paper”. Samples will be taken for analysis (e.g. hematologic and hemolytic analysis as described below).

By way of non-limiting example, RBCs can be thawed and analyzed as follows. The remainder of the cells can be deglycerolized in a polypropylene conical tube by serial dilutions. All saline additions can be added to the cell suspensions using a peristaltic pump at 10 ml/min while the cells can be thoroughly mixed on a shaker. Initially, 12% and 1.6% saline solutions can be added sequentially to the remaining sample at ratios of 3:10 (v/v) and 1:2 (v/v) to the sample, with a 5-min rest period after each addition. The cells can then be isolated by centrifuge at 2000 rpm. This saline addition, rest, and isolation cycle will be repeated first with 1.6% saline and finally by 0.9% saline at a ratio of 2:3 (v/v) to the original sample. After final centrifuge, the cells can be resuspended to a final hematocrit of 50% with 0.9% saline/0.2% glucose⁴². All supernatants from the centrifugation steps can be saved and combined to determine the amount of hemoglobin lost during the deglycerolization procedure. The deglycerolized RBC suspensions can be further checked for hematologic properties and functional characterizations. These experiments can be repeated multiple times to verify that the vitrification and thawing process is repeatable. The thawing methods for RBCs can be optimized to ensure high cell viability/functionality.

The system described herein can be applicable to other blood products such as platelets, white blood cells, and serum.

Characterizing the Vitrified/Thawed Red Blood Cells.

To characterize the post-thawed RBCs, in vitro measurements such as hematocrit, free hemoglobin and LDH can be used to assess hemolysis, P₅₀, 2,3-diphosphoglycerate (2,3-DPG), Na⁺ and K⁺ levels, S-nitrosohemoglobin (SNO-Hb) levels and RBC deformability studies.

Hemolysis:

Hematologic analyses, as described above herein, of the post-thawed RBCs can be carried out to examine the functionality and viability of the cells.

Osmotic Fragility Analysis.

A standard osmotic fragility method utilizing saline solutions ranging from 0.1 to 0.9% can be employed to determine the ability of the cells to withstand hypotonically induced water flux, which leads to hemoglobin leakage and cell lysis. RBC suspensions can be diluted at a ratio of 1:100 in each saline solution within the range, and then mixed, and allowed to incubate at room temperature. Following the incubation period, the samples can be mixed again to re-suspend any settled RBCs. The fraction of hemolyzed cells can be calculated by determining the ratio of free hemoglobin to the total hemoglobin concentration using a spectrophotometric measurement. Free hemoglobin introduced as part of the original RBC suspension can be subtracted prior to the hemolysis calculation so that the osmotic fragility curves represent only hemolysis due to the dilution in the saline solutions. The fragility index of the cell suspension can be used to quantify the degree of fragility. A larger fragility index corresponds to a more fragile cell.

RBC Protein Analysis.

Protein analysis can be conducted based on a cytoskeletal protein, spectrin, which lines the intracellular side of the RBCs' plasma membrane in a hexagonal arrangement. RBC ghosts and hemoglobin-depleted cytosol samples can be prepared to isolate spectrin and to analyze intracellular proteins not associated with the cytoskeleton, respectively. Purified protein samples will be separated through electrophoresis. Electrophoresis gels can be either stained with Coomassie blue for total protein analysis or blotted onto a polyvinylidene difluoride membrane for Western blot analysis. Densitometry analysis for Coomassie stained gel can be carried out with the Quantity One quantitation software (Bio-Rad, Hercules, Calif.).

It should be noted that a major advantage of cell-encapsulating droplet vitrification over the standard methods is the potential for scalability with a reduced use of CPAs. One critical point for such a system to be clinically viable is demonstration of the high throughput nature and scalability of cell-encapsulated droplet vitrification. Described elsewhere herein is a scalable prototype of 25 ejectors (5×5) and a demonstration of high throughput vitrification of blood (25 ml in 5 minutes). Described in this Example are systems and methods for integration of microfluidic and nanodroplet based approaches, with a broadly applicable technology to multiple other cell types including germ cells and stem cells.

### REFERENCES

1. Fuller B, Lane N, Benson E E, eds. Life in the Frozen State: CRC Press; 2004.
2. Standards for blood banks and transfusion services. QRB Qual Rev Bull 1977; 3:17,22.
3. Hess J R, Greenwalt T G. Storage of red blood cells: new approaches. Transfus Med Rev 2002; 16:283-95.
4. Hogman C F. Preparation and preservation of red cells. Vox Sang 1998; 74 Suppl 2:177-87.
5. Song Y S, Adler D, Xu F, et al. Vitrification and levitation of a liquid droplet on liquid nitrogen. Proc Natl Acad Sci USA 2010; 107:4596-600.
6. Scott K L, Lecak J, Acker J P. Biopreservation of red blood cells: past, present, and future. Transfus Med Rev 2005; 19:127-42.
7. Meryman H T, Hornblower M. A method for freezing and washing red blood cells using a high glycerol concentration. Transfusion 1972; 12:145-56.
8. Tullis J L, Ketchel M M, Pyle H M, et al. Studies on the in vivo survival of glycerolized and frozen human red blood cells. Journal of the American Medical Association 1958; 168:5.
9. Rowe A W, Eyster E, Kellner A. Liquid nitrogen preservation of red blood cells for transfusion; a low glycerol-rapid freeze procedure. Cryobiology 1968; 5:119-28.
10. Pert J H, Schork P K, Moore R. Low Temperature preservation of human erythrocytes. Biochemical and clinical aspects. Bibliotheca haematologica 1964; 19:7.
11. Krijnen H W, Wit J J F M D, Kuivenhoven A C J, Loos J A, Prins H K. Glycerol treated human red cells frozen with liquid nitrogen. Vox Sang 1964; 9:13.
12. Wolstenholme G E W, O'Connor M, eds. The Frozen Cell. London Churchill; 1970.
13. Zadeoppe A M M. Posthypertonic hemolysis in sodium chlorid systems. Acta Physiol Scand 1968; 73:341.
14. Pegg D E, Diaper M P. The effect of initial tonicity on freeze/thaw injury to human red cells suspended in solutions of sodium chloride. Cryobiology 1991; 28:18-35.
15. Pegg D E, Diaper M P. On the mechanism of injury to slowly frozen erythrocytes. Biophys J 1988; 54:471-88.
16. Lovelock J E. The denaturation of lipid-protein complexes as a cause of damage by freezing. Proc R Soc Lond Ser B-Biol Sci 1957; 147:427-33.
17. Mazur P, Leibo S P, Chu E H. A two-factor hypothesis of freezing injury. Evidence from Chinese hamster tissue-culture cells. Exp Cell Res 1972; 71:345-55.
18. Fahy G M, MacFarlane D R, Angell C A, Meryman H T. Vitrification as an approach to cryopreservation. Cryobiology 1984; 21:407-26.
19. Luyet B J, Gehenio P M. The mechanism of injury and death by low temperature. Biodynamica 1940; 3:67.
20. Rivals Race for U. S. Approvals on Blood Substitutes. Knight Ridder/Tribune Business News 2001.
21. Song Y S, Moon S, Hulli L, Hasan S K, Kayaalp E, Demirci U. Microfluidics for cryopreservation. Lab Chip 2009; 9:1874-81.
22. Fadini R, Brambillasca F, Renzini M M, et al. Human oocyte cryopreservation: comparison between slow and ultrarapid methods. Reprod Biomed Online 2009; 19:171-80.
23. Schmidt P J. Blood and disaster-supply and demand. N Engl J Med 2002; 346.
24. Wagner C T, Martowicz M L, Livesey S A, Connor J. Biochemical stabilization enhances red blood cell recovery and stability following cryopreservation. Cryobiology 2002; 45:153-66.
25. Pert J H, Schork P K, Moore R. Low-temperature preservation of human erythrocytes: biomedical and clinical aspects Bibliotheca haematologica 1964; 19:7.
26. Klein H G, Amstee D J, eds. Mollison's Blood Transfusion in Clinical Medicine. 11 ed: Wiley-Blackwell; 2006.
27. Fridey S L, ed. Standards for blood banks and transfusion services. 25 ed. Bethesda, Md.: American Association of Blood Banks; 2008.
28. Zadeoppe A M M. Posthypertonic hemolysis in sodium chlorid systems. Acta Physiologica Scandinavica 1968; 73:341.
29. Hess J R. Red cell freezing and its impact on the supply chain. Transfus Med 2004; 14:1-8.
30. Moon S, Lin P A, Keles H O, Yoo S S, Demirci U. cell encapsulation by droplets. J Vis Exp 2007:316.
31. Demirci U. Droplet-based photoresist deposition. Applied Physics Letters 2006; 88.
32. Demirci U, Montesano G. Cell Encapsulating Droplet Vitrification. Lab Chip 2007; 7:1428-33.
33. Hong J W, Quake S R. Integrated nanoliter systems. Nat Biotechnol 2003; 21:1179-83.
34. Okamoto T, Suzuki T, Yamamoto N. Microarray fabrication with covalent attachment of DNA using bubble jet technology. Nat Biotechnol 2000; 18:438-41.
35. Utada A S, Lorenceau E, Link D R, Kaplan P D, Stone H A, Weitz D A. Monodisperse double emulsions generated from a microcapillary device. Science 2005; 308:537-41.
36. Arav A, Yavin S, Zeron Y, Natan D, Dekel I, Gacitua H. New trends in gamete's cryopreservation. Mol Cell Endocrinol 2002; 187:77-81.
37. Parkening T, Tsunoda Y, Chang M. Effects of various low temperatures, cryoprotective agents and cooling rates on the survival, fertilizability and development of frozen-thawed mouse eggs. J Exp Zool 1976; 197:369-74.
38. Karlsson J O M, Toner M, eds. Cryopreservation. San Diego: Eds. Academic Press; 2000.
39. Ludwig M, Al-Hasani S, Felderbaum R, Diedrich K. New aspects of cryopreservation of oocytes and embryos in assisted reproduction and future perspectives. Hum Reprod 1999:162-85.
40. Stachecki J J, Cohen J. An overview of oocyte cryopreservation. Reprod Biomed Online 2004; 9:152-63.
41. Horn E P, Sputtek A, Standl T, Rudolf B, Kuhnl P, Shulte E J. Transfusion of autologous, hydroxyethyl starch-cryopreserved red blood cells. Current Researches in Anesthesia & Analgesia 1997; 85:739-45.
42. Wagner C T, Burnett M B, Livesey S A, Connor J. Red blood cell stabilization reduces the effect of cell density on recovery following cryopreservation. Cryobiology 2000; 41:178-94.

### Example 4

### Second Grant Minimizing the Role of Cryoprotectant Toxicity for Cryopreservation

Long-term preservation of cells and tissues through novel cryo-technologies has broad impacts in multiple fields including tissue engineering, regenerative medicine, stem cells, blood banking, animal strain preservation, clinical sample storage, transplantation medicine and in vitro drug testing. Specifically, in modern clinical medicine, germ cell cryopreservation offers an option to extend human fertility.

Vitrification (ice/crystal-free cryopreservation) has emerged as a novel approach over traditional slow freezing. Although vitrification minimizes mechanical damage due to ice crystal nucleation, it suffers from toxicity due to high concentrations of cryoprotectant agents (CPAs). The high CPA levels lead to two main challenges. (i) Sudden cell volume changes driven by the osmotic flow and mass transport can damage the cells during addition and removal of the CPAs. (ii) Multiple CPA addition and removal steps are required. These lengthy manual processing steps add to the technical complexity, require highly trained technicians, and cause variations among users. This proposal investigates a new experimental strategy to minimize the CPA concentrations facilitated by theoretical understanding of the underlying mechanisms to improve the clinical outcomes using novel technologies.

CPA addition and removal has been a significant problem since vitrification emerged as an effective preservation strategy. Described herein are methods and systems relating to reduced CPA levels in minimal volume vitrification integrated with gradual CPA addition/removal steps to significantly reduce the toxicity and osmotic damage on cells undergoing cryopreservation, hence, resulting in enhanced overall post-thaw viability and functionality. CPA levels can be decreased by decreasing the droplet volumes below hundreds of nanoliters using the method and systems described herein. The methods and systems described here further minimize the toxicity and osmotic-driven cell volume changes by reducing the required CPA concentrations and still achieving successful vitrification. Additionally, the method and systems described herein will leverage microfluidic technologies to eliminate the need for multiple solution changes and manual handling steps for CPA addition and removal. There is a need to minimize cell-channel wall interactions to avoid spindle and chromosomal damage for larger cells like oocytes. Described herein is a novel device which can replace the cell-flow based systems, where the cells will be kept in a stationary chamber that is connected to the main channel by a porous membrane. This system can expose the cells to CPAs gradually through diffusion.

CPA Addition/Removal Causes Cell Damage.

During the addition and removal of CPAs, osmotic transport of water across the cell membrane and cell volume changes are observed. If these volume changes are excessive, they can cause damage^{12, 45-48}. Each cell type has their own osmotic tolerance limit. The osmotic shock is more apparent at high CPA concentration gradients indicating the need for vitrification at low CPA concentrations. In addition to osmotic damage, CPA exposure can result in cell damage due to the chemical toxicity at high CPA levels. These toxic effects depend on the cell and CPA type, duration of exposure, concentration, and can be monitored through viability and functional assays^{25, 26, 42, 49-53}. Hence, there is a need to understand quantitatively the CPA addition/removal steps, so the CPA levels and associated toxicity and osmotic damage can be minimized with novel technologies.

The systems and methods described herein relate to a fundamentally new approach for minimizing toxicity during CPA addition and removal. This proposal offers multiple innovative technological advantages as follows. First, it takes a pioneering step to introduce picoliter to nanoliter level fluid handling and cell manipulation technologies, and concepts to cryopreservation. Nanoliter droplet-based, ultra-rapid freezing method offers ultra-rapid cooling, and fast warming rates that allow CPA concentrations as low as those utilized in conventional slow freezing protocols. The strategy described herein is to improve the quality of frozen cells by achieving extremely rapid vitrification using nanoliter droplets at very low CPA levels. The vitrification procedure described herein would also prevent direct chilling injury to plasma membrane and meiotic oocyte spindles due to ultra high cooling rate (on the order of −10,000° C./sec). In addition, it would significantly minimize osmotic damage to plasma membrane and other vital subcellular structures, and avoid CPA toxicity due to much lower CPA concentration (1-3M) than previous vitrification procedures (6-8M). Second, current manual CPA additional/removal protocols cause osmotic and mechanical stresses on the cells. A combination of microfluidic addition/removal of low CPA concentrations, and a minimum level of handling reduces osmotic effects and mechanical stress on the cells⁶⁰. These effects often occur with the existing cryopreservation protocols when cells are manually transferred from one CPA solution to another. Third, this decreases the manual transfer steps and burden on the embryology laboratory. Fourth, the diminishing manual handling steps decreases handling errors and test-to-test variability. Hence, by applying these nano/microscale technologies to develop a novel method, a solution is provided for the acute needs of cryopreservation by improving cell survival and function. Fifth, the developed system can be simple, and easy to use. It can utilize simple microfluidic systems that are free of complex valving or expensive peripherial equipment. These systems can be designed to be robust and suitable for routine use in clinical laboratories to significantly improve the efficiency of a frozen embryo transfer cycle. The commercial and translational potential of this platform is high, since it could perform some of the routine processes of an embryology laboratory the same way every time by automation diminishing the operator variability. Sixth, the experimental investigations are coupled with theoretical modeling to predict cell membrane mass transfer forming a quantitative CPA addition and removal model. Also, the proposed modeling aspects provide a quantitative framework for understanding the experimental outcomes and optimal conditions, which would be a huge step forward over the existing empirical approaches. The methods and systems described herein enable new vitrification strategies and improved protocols.

Minimum Achievable Vitrification Concentrations with Various CPA and Cell Types.

High CPA concentrations raise the effective glass transition temperature, thereby reducing the probability of ice crystal formation. However, using high CPA levels brings together osmotic shock and chemical toxicity for the cells leading to an overall reduction in post-thaw viability. Thus, it is crucial to identify the minimum CPA concentration required to ensure the occurrence of vitrification with reduced ice crystal formation and chill injury. The CPA types and cell type can be varied to identify the lowest CPA concentrations that can be used to achieve vitrification. For mouse oocytes and embryonic stem cells (v6.5), the following commonly used CPA types can be evaluated: (i) ethylene glycol (EG), (ii) dimethyl sulfoxide (DMSO), and (iii) 1,2-propanediol (PrOH) combined with sucrose at varying concentrations ranging from 0.5 M to 6 M, with 0.5 M increments^{71, 72}. Alternatively, for sperm vitrification glycerol can be evaluated at concentrations varying from 0% to 12% with 1% increments. The droplet ejection system, which generates precisely controlled size droplets and is described above herein, will be used to encapsulate different cell types in droplets for ejection into liquid nitrogen. In this system, cell suspension in pre-determined concentrations of CPAs can be introduced into the ejector through tubing connected to a syringe pump or a stripper. Then, the cell encapsulating CPA droplets can be directly ejected into liquid nitrogen. For each CPA concentration, various droplet sizes can be generated by adjusting operation conditions of the ejector system, such as the gas flow rate and the outflow rate of cell/CPA suspension. To determine these parameters, theoretical heat transfer models for vitrification will be used as described herein. The sizes and distribution of droplets can also be characterized to determine the optimal ejector operation conditions for each cell type. After the ejection of the droplets is completed, they can be collected on thin sheets of filter and kept over liquid nitrogen vapor as they are transferred to standard cryovials, which are pre-cooled in liquid nitrogen vapor to avoid thawing. The cryovials containing the vitrified cells then can be transferred into cryo-containers for storage. The vitrified droplets can be identified based on their translucent appearance. Then, the cryopreserved cells can be retrieved following the ultra rapid warming procedures and evaluated for viability and function^{71, 72} according to the methods that were described elsewhere herein. Based on the detailed viability and functionality analyses, the lowest possible CPA concentration and the CPA type for vitrification can be identified.

Minimizing Sample Volume.

CPA concentrations as low as possible can be obtained while maintaining vitrification by optimizing the CPA concentration, CPA addition/removal mechanism, and droplet size correlated to the degree of crystallization to suit the cell types of interest, i.e. oocytes, sperm, stem cells. It has been demonstrated herein above with oocytes that this can be achieved through droplet size optimization, where single oocytes can be encapsulated in droplets and then be vitrified. This presents the best case especially for oocytes since they have a larger size and more water content compared to other mammalian cells. For sperm cells, the droplet size can be chosen to be even smaller due the smaller size of sperm cells. Thus, the ideal droplet size would encapsulate single to few sperm in each droplet. Drop sizes can be explored to cover the size range to encapsulate murine oocytes (100-300 μm), sperm (50-300 μm) and stem cells (20-200 μm), or human oocytes, sperm or stem cells. Based on the theoretical understanding of vitrification event in the presence of cells encapsulated in droplets, the minimum CPA levels will be attained by choosing the optimal droplet size and cell concentration within droplets.

Experimental studies described in the preceding paragraph can be coupled to theoretical studies in order to: (i) estimating the initial conditions for the experiments, (ii) better interpret the experimental results, and (iii) design the next step of experiments. This iterative approach produces more rapid results in complex systems with many parameters. The inventors have previously demonstrated the vitrification and crystallization phenomena when a droplet is directly ejected into liquid nitrogen. The droplet vitrification phenomenon can be analyzed in the presence of encapsulated cells. The Stefan model and the zone model can be utilized for this evaluation, and the assumptions and governing equations for crystallization will be similar to those used for droplet vitrification and heat transfer without cells (Eqn3., Eqn. 4):

$$\frac{d χ}{d t} = {k}_{a} {χ}^{\frac{1}{2}} ( 1 - χ ) ( {T}_{m} - T ) {e}^{- Q / RT} , \qquad ( Eqn . 3 )$$

where kₐ is a characteristic constant, Tₘ the temperature at the end of freezing process, Q the activation energy, R the gas constant, and χ the degree of ice crystallization (0<χ<1). This zone model is based on the hypothesis that the sum of specific and latent heats can be given by an enthalpy function⁷⁶.

It is assumed that heat conduction plays a dominant role within the droplet in heat transfer but not heat convection, and heat transfer equation is expressed as:

$$\frac{\partial {T}^{*}}{\partial {t}^{*}} = {∇}^{2} {T}^{*} + St \frac{\partial χ}{\partial {t}^{*}} , \qquad ( Eqn . 4 )$$

where t_{c}=R²/α is characteristic time, r*=r/R is dimensionless radius, t*=t/t_{c}(=Fo) is dimensionless time (Fourier number),

$${T}^{*} = \frac{T - {T}_{x}}{{T}_{i} - {T}_{x}}$$

is dimensionless temperature, in which T₁ is the initial temperature of droplets, T_{∞} is the temperature of liquid nitrogen, and h is the convective heat transfer coefficient. ∇ is a differential vector operator and St denotes the Stefan number

$$St = \frac{L}{{c}_{p} ( {T}_{i} - {T}_{x} )}$$

(L is the heat of fusion). The boundary conditions are:

$$\frac{\partial {T}^{*}}{\partial {r}^{*}} = 0 , at {r}^{*} = 0 and \frac{\partial {T}^{*}}{\partial {r}^{*}} = - {BiT}^{*}$$

at r*==1, in which

$$Bi = \frac{hR}{k}$$

is the Blot number. The initial condition of T=Tᵢ at t=0 is applied. Spherical coordinates can be used in this theoretical modeling with assumptions including incompressible fluids, laminar and axisymmetric vapor flow, temperature-independent viscosity and density, negligible viscous heating, and smooth surface. The continuity and momentum equations can be solved based on the assumptions listed above, and more details can be found in the inventors' previously published work³⁹.

The volume fraction of cells to the total droplet volume can be determined by the cell types used, such as a single oocyte, single to few stem cells, and single or multiple sperm cells per droplet. Based on the models listed above, it is anticipated that higher efficiency will be evidenced compared to the controls (i.e., straws and loop methods), given that cells do not significantly differ in heat resistance than medium or CPAs.

Minimize Toxicity and Osmotic-Driven Cell Volume Changes During CPA Addition and Removal.

Stepwise CPA addition/removal methods aim to reduce osmotic effects by keeping the osmotic-driven cell volume changes within the tolerance limits. However, the inventors have shown that the stepwise changes in concentration can still cause steep transport gradients through the cell membrane causing cell damage⁴⁸. Described herein is a system that leverages the microfluidic control over CPA concentrations with a stationary chamber for gradual CPA addition and removal to reduce the osmotic effects and toxicity. In addition, the system can be optimized based on the theoretical understanding of CPA and water transport through cell membrane.

A Chamber with Dynamic Control Over CPA Concentrations During Addition and Removal Steps.

The inventors have shown that by employing gradual CPA changes in microfluidic channels, the osmotic effects can be minimized and cell viability can be increased up to 25% with hepatocytes⁴⁸. However, the challenge with these systems is that the cells need to travel through long serpentine microchannels. This may lead to mechanical shear on the cells due to interactions with microfluidic walls during the flow, if the channel dimensions and flow rates are not carefully optimized especially considering the oocytes that are large in size and extremely sensitive to mechanical effects. Also, the inventors have demonstrated that multiple fluids can be precisely and rapidly mixed using microfluidic technologies. Therefore, the flow rates of CPA and the cell media introduced through the channel inlets will be controlled to achieve gradual CPA concentration increase/decrease to the cells residing in a cell chamber instead of flowing them through long channels.

Design Considerations of Chamber-Based Microfluidic Devices for CPA Addition/Removal:

Described herein is a chamber-based microfluidic system, where cells will be kept in a stationary chamber that is connected to the main channel by a porous membrane, as described in FIG. 15 and Song et al. Lab Chip 2009; 9:1874-81. The device can have two microfluidic layers with a porous membrane sandwiched between the two layers, (i) exposure chamber, and (ii) gradual CPA concentration control channel. The two layers can be strengthened by double-sided adhesive film to avoid leakage between the layers. Such devices earlier at high flow rates (>100 μL/min) with unprocessed whole blood⁶⁸. The cells will be held within the exposure chamber located at the end of the gradual CPA concentration channel (FIG. 15). The pore sizes of the membrane will be kept below 5 μm, since this pore size has been experimentally validated to allow CPA diffusion and prevents cells (as small as 10 μm diameter) from passing through the membrane (see Song et al. Lab Chip 2009; 9:1874-81). The cells can be loaded to and unloaded from the exposure chamber through a separate inlet and outlet than the CPA channel. The CPA concentration in the channel can be precisely controlled by adjusting flow rates with microliter per second resolution at the inlets utilizing syringe pumps (Harvard Instruments, MA). Channel length and flow rates can be optimized for rapid, efficient fluid mixing, and diffusive transport to the cells inside the chamber. To ensure that CPA and cell media have been well mixed before exposure to the cells, the channel length can be designed and optimized through a mathematical diffusion model as described in Song et al. Lab Chip 2009; 9:1874-81. Using this model, (a) the time for CPA exposure to cells within 5 seconds, and (b) the final CPA concentration exposed to the cells can be controlled. Devices can be fabricated with channel lengths ranging from 1 to 30 cm, allowing cells to experience gradual CPA changes. The flow rates can range from 1 to 50 μl/min, providing a broad window to optimize the system. The cell chamber can be designed to be 2 mm×2 mm×250 μm to accommodate the large size of oocytes. Oocytes can be flowed through 60 cm long microchannels (150 μm high×200 μm wide) after which the cells had intact spindles and they were viable and functional (Song et al. Lab Chip 2009; 9:1874-81). Therefore, comparable width and height can be used for channels connecting the cell exposure chamber to the ports. These channels will be significantly shorter (<2 cm).

Fabrication and Operation of Microfluidic CPA Addition/Removal Channels.

The microfluidic devices can be fabricated using PMMA, considering the advantages in rapid prototyping for varying channel sizes and geometries. PMMA is widely used for biological experiments by other researchers in the microfluidics field. Alternatively, glass/glass microchannels can be fabricated. The time spent for CPA addition/removal in the chip can be designed to be comparable to the time spent by current methods. For instance, manually transferring oocytes drop-to-drop for addition and removal of CPAs is a 5 to 10 minute process. The inventors' work demonstrates that the microfluidic process adds and removes CPAs within similar time durations compared to the standard manual clinical protocols. In addition, higher throughput can be achieved with this microchip by applying up to 60 oocytes at once, while the standard manual method can only handle a few oocytes each time.

Comparing the Chamber-Based Microfluidic System to Current CPA Addition/Removal Approaches.

Standard one-step or stepwise CPA addition/removal methods (for oocytes) and centrifugation methods (for stem cells and sperm) can be used as benchmark controls to test how the system improves the cell viability by reducing the osmotic shock and toxicity due to CPA exposure. The speed of cell shrinkage and expansion during CPA addition/removal will be observed under a microscope. These comparisons can allow the identification of the optimal procedures for CPA addition and removal for each cell type to minimize osmotic shock. For CPA removal, the same microfluidic chamber-based approach can be used, while the concentration of CPAs exposed to the cells can be gradually decreased by adjusting the CPA solution and cell media at channel inlets.

Understand and Optimize the Mass Transport Through Cell Membrane During CPA Addition and Removal.

The inventors have theoretically demonstrated that the microfluidic system reduced the sudden water and CPA flux across the cell membrane compared to standard one-step and step-wise CPA addition/removal methods. This result was also confirmed experimentally using hepatocytes. Using these theoretical principles, the chamber-based microfluidic system can be modeled to identify the optimal channel design and operation procedures. The flow behavior of CPA and cell media in the microfluidic channel can be modeled using the steady state Navier-Stokes equations:

ρu·∇u=∇·η(∇u+(∇u)^{T})−∇P, and ∇·u=0 (5)

where ρ and η represents the fluid density and viscosity, which are assumed to be linear functions of CPA concentration (rule of mixture). In addition, u denotes the velocity vector and P is the fluid pressure. Mass transport for a single species will be taken into account assuming that CPAs interact only with water molecules and physical properties count only on the CPA concentration. The transport phenomenon of CPAs is governed by the following convection and diffusion equation at steady state:

∇·(cu)=∇·(D∇c) (6)

in which c is the concentration of CPAs and D indicates the diffusion coefficient of CPAs. As a boundary condition, an average velocity can be imposed at the inlet, which develops laminar flow in the microfluidic channel. At the outlet, the pressure can be set to zero (atmospheric pressure). Also, no-slip condition (u=0) will be applied to all other boundaries. For mass balance, the concentration boundary condition can be given to the inlets. In addition, the convective flux condition can be imposed at the outlet, which means that mass diffusion in the normal direction to the outlet surface is negligible.

For the modeling of mass transport across cell membrane, Kedem-Katchalsky model (a three-parameter model) can be used as it is widely accepted as a model that closely simulates mass transport through the cell membrane. In this model, the water and solute transport across a membrane is mathematically coupled by using the reflection coefficient. Depending on the reflection coefficient, water and CPA are transported through a common channel. This mathematical modeling can provide insight into fluid fluxes, volume changes, and CPA molarity of cells due to osmotic pressure. The water flux across the cell membrane is expressed as below:

$${J}_{w} = {L}_{p} Δ P - {L}_{p} - {L}_{p} σ RT Δ c , and \\ {P}^{i} - {P}^{e} = E \frac{V - {V}_{0}}{{V}_{0}} + {P}_{0}^{i} - {P}_{0}^{e} , where \\ \frac{d {V}_{w}}{d t} = {J}_{w} A \qquad ( 7 )$$

where Lp is the membrane hydraulic conductivity, σₛ the membrane reflection coefficient of CPAs, R the universal gas constant, T the temperature, c the concentration of CPAs, A the cell surface area, E the cellular elastic modulus and v_{w} the water volume of cells. In addition, the superscripts i and e mean the intracellular and extracellular regions, respectively. Secondly, the CPA transport is modeled by the following equations:

$${J}_{c} = ( 1 - σ ) {c}_{up} {J}_{w} + ω RT Δ c , and \\ \frac{d {V}_{c}}{d t} = {J}_{c} A \qquad ( 8 )$$

where ω is the membrane permeability of CPAs and cup is the upstream concentration of CPAs according to the water flux direction. The 4th order Runge-Kutta method can be implemented to calculate equations.

Cryopreservation Outcome Through Performing Viability and Functional Assays.

Merge Droplet Ejection and CPA Addition/Removal Systems for Minimum Volume Droplet Vitrification.

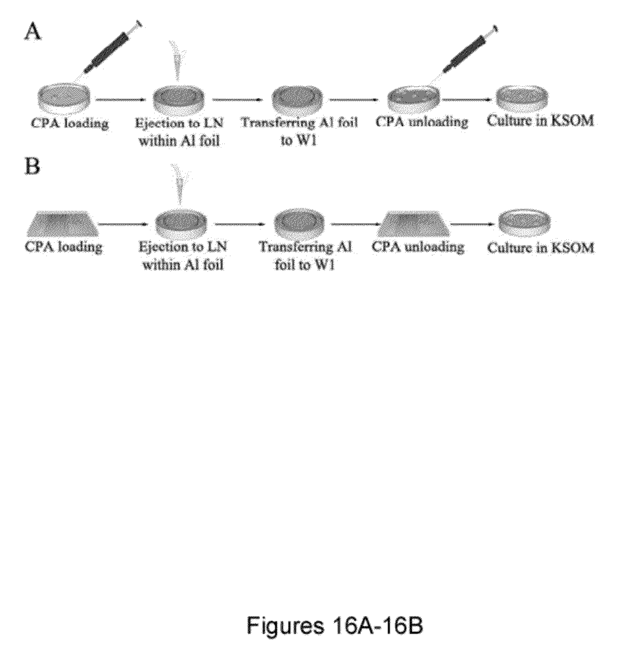

The standard oocyte and sperm collection procedures have been established in our laboratory under the animal protocol (#04444) as described by Hogan⁷⁷ and Liu⁷⁸. For CPA addition, oocytes, sperm or stem cells can be initially introduced into the porous membrane chamber from cell chamber inlet. The CPA at selected concentrations and cell media can be introduced (FIG. 15) into the channel inlets using syringe pumps by programmable adjustments to the flow rates at the inlets. After CPA addition, the cells can be transferred to the ejector through PE tubing, which connects the cell chamber outlet and stripper tip of the ejector (FIG. 6A) establishing a reliable interface. This process can be actuated using a syringe filled with fresh vitrification solution connected to the cell chamber inlet. After this transfer step, the cells to be ejected can be loaded to the stripper tip. The ejector can be composed of a 125 μm stripper tip (MidAtlantic, NJ) that is located at a 200 μl pipette-tip center. TYGON tubing (ID=3.2 mm) can be used to connect the ejector to the gas source. Nitrogen gas can flow through the pipette tip while CPA-cell solution flows through the stripper tip. This process creates cell encapsulating CPA droplets, which are then ejected into liquid nitrogen (FIGS. 16A-16B). The inventors have successfully interfaced and merged the microfluidics and droplet generation systems and tested the overall system for oocytes as described above herein. At the microfluidic-ejector interface, the connecting tubing provides a continuous flow channel preventing potential cell settlement. The merged ejector and microfluidic system can be set at a distance (>5 cm) from liquid nitrogen to avoid any ice formation in the ejector or microchannels. Then, the vitrified droplets can be retrieved using a cell strainer. This strainer can be fit into a standard 1.5 mL polypropylene cryo-vial for long-term closed storage in liquid nitrogen. As an alternative approach, the cell-encapsulated droplets can be ejected onto a PE film first, and then immersed into liquid nitrogen to avoid any potential droplet aggregation. To measure the droplet size, the droplets can be ejected onto a transparent film and measured using a microscope or by standard stroboscopic imaging described above herein⁷⁹. To evaluate the ejection step, for example, oocytes from Female B6D2F1 mice aged 6 to 8 weeks old (Jackson Laboratory, ME) can be ejected into medium and evaluated for survivability and function.

Ultra Rapid Warming Procedures.

Rapid thawing of vitrified cells is as important as rapid vitrification and follows similar heat transfer physics as described earlier. Nanoliter droplets enable high heat transfer rates during the rapid thawing process. For the warming step, vitrified cells can be retrieved from the cryo-vials by removing the cell strainer using cooled, sterile forceps, and rapidly transferring into warm thawing solution (37-40° C.) along with warming solution added into the strainer. Surrounding nitrogen vapor facilitates successful transfer of the strainer before immersion into the thawing medium. After thawing, the cells can be allowed 2 minutes for recovery from the shrunken state that they are in before CPA removal. Cells can be transferred into the chamber pre-filled with thawing solution. CPAs can then be removed by gradually introducing decreased concentrations of CPAs to the cells by adjusting the flow rates of initial CPAs and media. After completely removing the CPAs, the cells can be transferred to warm culture medium for culture and functional evaluation. All steps including ejection will be performed in a sterile, humidity and temperature controlled environment.

Cells can be analyzed for viability and function. By way of non-limiting example, oocytes can be analyzed as follows.

Analysis for Oocytes:

From an embryological point of view, harvested oocytes can be classified into three stages of development: GV, M-I and M-II. Experiments can be performed initially with mature oocytes, since they are known to be more compatible with freezing protocols⁶, and also these are the oocytes usually processed in clinical IVF settings. Oocyte viability can be evaluated after culturing in KSOM medium supplemented with 1% BSA for 24 and 48 hours based on cell morphology. For instance, whether the cytoplasm looks refringent and there is sign of degeneration can be checked. The parthenogenetic activation and following embryo development can be used to assess oocyte function. The thawed oocytes can be incubated in 50 mM SrCl₂ in KSOM medium for 2 hours, and followed with a five-time wash⁸⁰. Following activation, the oocytes can be cultured in calcium-free Chatot Ziomek Bavister (CZB) medium supplemented with 1% BSA and overlaid with mineral oil (Sigma) in Petri-dishes in a 5% CO₂ incubator at 37° C. The percentage of embryos reaching 2- and 4-cell stages after 24 and 48 hours, and morula and blastocyst stages after 72 and 120 hours in culture can be counted. In addition, the oocyte spindle and chromosome integrity after vitrification/thawing can be assessed using immunostaining⁸¹. Fresh oocytes can be used as controls for each experiment. Fixation and all subsequent incubations can be carried out at 37° C. The oocytes can be stained for α-tubulin and PI (propidium iodide) to observe the mechanical effects on the spindles and the chromosomes. The localization of tubulin and chromatin revealed by FTIC and PI fluorescence can be observed.

Alternatively, stem cells can be analyzed as follows: Briefly, the stem cell membrane integrity after thawing can be analyzed using Live/Dead® viability assay (calcein EM and ethidium homodimer). For long term viability, cell attachment assay can be performed after a day and proliferation assays can be performed over a three day observation period as described elsewhere⁵³. To determine whether the embryonic stem cells retained the undifferentiated properties as pluripotent cells post cryopreservation, the expression of transcription factor Oct-4, the expression of membrane surface glycoprotein SSEA-1, the expression of Nanog, and the elevated expression of the enzyme alkaline phosphatase, can be analyzed with PCR and immunostaining. In vitro differentiation assays, such as embryoid body⁶⁷, and teratoma formation can be performed.

Alternatively, sperm can be analyzed as follows: sperm after thawing can be immediately checked for motility. The kinematic parameters that define sperm motility, including curvilinear velocity (VCL), straight-line velocity (VSL) and linearity (LIN=VSL/VCL) can be calculated⁶⁹. VCL refers to the distance that the sperm head covers during the observation time. VSL is referred to the straight-line distance between the starting and the ending points of the sperm trajectory. The percentage of motile sperm, defined as the fraction of motile sperm relative to the total sperm count, can also be analyzed as previously described⁶⁹.

Comparisons of the Proposed Droplet Vitrification Platform with Existing Methods.

The combined chamber-based CPA addition/removal microfluidic device and droplet vitrification system described herein can be compared with existing methods to reveal the advantages of the presently described system for cryopreservation. For instance, Straw²⁷⁻³⁰ and loop-based^{82, 83} vitrification methods can be used for oocyte cryopreservation as controls. Slow freezing methods can be used as controls for stem cells and sperm freezing. To further assess the maintenance of normal function of thawed germ cells, fertilization of survived oocytes using fresh sperm or survived sperm with fresh oocytes using conventional murine IVF technologies can be performed. The putative zygotes can be checked at 24, 48, and 72 hour intervals for evidence of fertilization and normal embryonic development based on usual morphological criteria. Rates of survival, fertilization, cleavage, and blastocyst formation can be compared.

Applying the Technology to Human Cells:

The system that is described herein is applicable to human cells. As an example, the human oocytes are on average 120 μm in diameter⁸⁴, whereas the murine oocytes are on average 80 μm in diameter⁸⁴. Both diameters are within the range of vitrifiable droplet sizes (<300 μm). Hence, these size differences will be taken into consideration, when designing the droplet sizes and resulting freezing kinetics. The channel dimensions of 250 μm×250 μm can be applied for both murine and human oocytes. The variability of oocyte organelle and membrane water permeability will require an overall protocol optimization to minimize the osmotic shock as described above herein.

Pertinent to future clinical applications, the sterilization of each component of the ejection system and conducting vitrification experiments in a sterile hood is established. The ejector, TYGON® tubing and alumina foil can be sterilized using autoclave. The nitrogen gas from the gas cylinder can be sterilized by flow through a 0.22 μm filter before reaching the ejector. The sterilization of liquid nitrogen can be accomplished by using sterile PTFE cartridge filters⁸⁵ and also by ultra-violet (UV) radiation as commonly done in the literature⁸⁶. The use of sterile liquid nitrogen minimizes potential contamination⁸⁷.

The systems and methods described herein have applications in tissue engineering, regenerative medicine, stem cells, blood banking, animal strain preservation, clinical sample storage, transplantation medicine and in vitro drug testing as well as reproductive medicine fields. The systems and methods described herein represent a simplification of existing protocols for the end user, e.g. the systems and methods described herein allow fully automated fluid handling and ejection by pushing a button to lower the technical requirements and skills so that this technique can be easily delivered to clinical labs.

### REFERENCES

1. Mazur P. Freezing of living cells: mechanisms and implications. Am J Physiol 1984; 247:C125-42.
2. Parkening T, Tsunoda Y, Chang M. Effects of various low temperatures, cryoprotective agents and cooling rates on the survival, fertilizability and development of frozen-thawed mouse eggs. J Exp Zool 1976; 197:369-74.
3. Agca Y. Cryopreservation of oocyte and ovarian tissue. ILAR J 2000; 41:207-20.
4. Agca Y, Liu J, Rutledge J J, Critser E S, Critser J K. Effect of osmotic stress on the developmental competence of germinal vesicle and metaphase II stage bovine cumulus oocyte complexes and its relevance to cryopreservation. Mol Reprod Dev 2000; 55:212-9.
5. Agca Y, Liu J, Peter A T, Critser E S, Critser J K. Effect of developmental stage on bovine oocyte plasma membrane water and cryoprotectant permeability characteristics. Mol Reprod Dev 1998; 49:408-15.
6. Stachecki J J, Cohen J. An overview of oocyte cryopreservation. Reprod Biomed Online 2004; 9:152-63.
7. Oktay K, Cil A P, Bang H. Efficiency of oocyte cryopreservation: a meta-analysis. Fertil Steril 2006; 86:70-80.
8. Stachecki J J, Cohen J. An overview of oocyte cryopreservation. Reprod Biomed Online 2004; 9:152-63.
9. Chen C. Pregnancy after human oocyte cryopreservation. Lancet 1986; 1:884-6.
10. Ludwig M, Al-Hasani S, Felderbaum R, Diedrich K. New aspects of cryopreservation of oocytes and embryos in assisted reproduction and future perspectives. Hum Reprod 1999:162-85.
11. Zhang X, Catalano P N, Gurkan U A, Khimji I, Demirci U. Emerging Technologies in Medical Applications of Minimum Volume Vitrification. Nanomedicine 2011; In press.
12. Kashuba Benson C M, Benson J D, Critser J K. An improved cryopreservation method for a mouse embryonic stem cell line. Cryobiology 2008; 56:120-30.
13. Ware C B, Nelson A M, Blau C A. Controlled-rate freezing of human ES cells. Biotechniques 2005; 38:879-80, 82-3.
14. Anchan R M, Quaas P, Gerami-Naini B, Bartake H, Griffin A, Zhou Y, Day D, Eaton J, George L L, Naber C, Turbe-Doan A, Park P J, Hornstein M D, Maas R L. Amniocytes can serve a dual function as a source of iPS cells and feeder layers. Human Molecular Genetics 2011.
15. Boldt J, Tidswell N, Sayers A, Kilani R, Cline D. Human oocyte cryopreservation: 5-year experience with a sodium-depleted slow freezing method. Reprod Biomed Online 2006; 13:96-100.
16. Chen S U, Lien Y R, Chen H F, Chang L J, Tsai Y Y, Yang Y S. Observational clinical follow-up of oocyte cryopreservation using a slow-freezing method with 1,2-propanediol plus sucrose followed by ICSI. Hum Reprod 2005; 20:1975-80.
17. Chi H J, Koo J J, Kim M Y, Joo J Y, Chang S S, Chung K S. Cryopreservation of human embryos using ethylene glycol in controlled slow freezing. Hum Reprod 2002; 17:2146-51.
18. Karlsson J O, Toner M. Long-term storage of tissues by cryopreservation: critical issues. Biomaterials 1996; 17:243-56.
19. Liu B, McGrath J J. Effects of two-step freezing on the ultra-structural components of murine osteoblast cultures. Cryo Letters 2006; 27:369-74.
20. Pegg D E, Wusteman M C, Wang L. Cryopreservation of articular cartilage. Part 1: conventional cryopreservation methods. Cryobiology 2006; 52:335-46.
21. Oegema T R, Jr., Deloria L B, Fedewa M M, Bischof J C, Lewis J L. A simple cryopreservation method for the maintenance of cell viability and mechanical integrity of a cultured cartilage analog. Cryobiology 2000; 40:370-5.
22. Yang H, Jia X, Ebertz S, McGann L E. Cell junctions are targets for freezing injury. Cryobiology 1996:672-3.
23. Zieger M A, Tredget E E, McGann L E. Mechanisms of cryoinjury and cryoprotection in split-thickness skin. Cryobiology 1996; 33:376-89.
24. Fahy G M, Levy D I, Ali S E. somer emerging principles underlying the physical properties, biological action, and utility of vitrification solutions. Cryobiology 1987; 24:18.
25. Wusteman M C, Pegg D E, Robinson M P, Wang L H, Fitch P. Vitrification media: toxicity, permeability, and dielectric properties. Cryobiology 2002; 44:24-37.
26. Fahy G M, Wowk B, Wu J, Paynter S. Improved vitrification solutions based on the predictability of vitrification solution toxicity. Cryobiology 2004; 48:22-35.
27. Vajta G, Holm P, Kuwayama M, Booth P J, Jacobsen H, Greve T, Callesen H. Open Pulled Straw (OPS) vitrification: a new way to reduce cryoinjuries of bovine ova and embryos. Mol Reprod Dev 1998; 51:53-8.
28. Suo L, Zhou G B, Meng Q G, Yan C L, Fan Z Q, Zhao X M, Fu X W, Wang Y P, Zhang Q J, Zhu S E. OPS vitrification of mouse immature oocytes before or after meiosis: the effect on cumulus cells maintenance and subsequent development. Zygote 2009; 17:71-7.
29. Chen S U, Lien Y R, Chen H F, Chao K H, Ho H N, Yang Y S. Open pulled straws for vitrification of mature mouse oocytes preserve patterns of meiotic spindles and chromosomes better than conventional straws. Hum Reprod 2000; 15:2598-603.
30. Chen S U, Lien Y R, Cheng Y Y, Chen H F, Ho H N, Yang Y S. Vitrification of mouse oocytes using closed pulled straws (CPS) achieves a high survival and preserves good patterns of meiotic spindles, compared with conventional straws, open pulled straws (OPS) and grids. Hum Reprod 2001; 16:2350-6.
31. Hochi S, Akiyama M, Minagawa G, Kimura K, Hanada A. Effects of cooling and warming rates during vitrification on fertilization of in vitro-matured bovine oocytes. Cryobiology 2001; 42:69-73.
32. Martino A, Songsasen N, Leibo S P. Development into blastocysts of bovine oocytes cryopreserved by ultra-rapid cooling. Biol Reprod 1996; 54:1059-69.
33. Kim S H, Ku S Y, Sung K C, Kang M J, Kim S A, Kim H S, Oh S K, Jee B C, Suh C S, Choi Y M, Kim J G, Moon S Y. Simplified EM grid vitrification is a convenient and efficient method for mouse mature oocyte cryopreservation. Yonsei Med J 2006; 47:399-404.
34. Tominaga K. Cryopreservation and sexing of in vivo- and in vitro-produced bovine embryos for their practical use. J Reprod Dev 2004; 50:29-38.
35. Lane M, Schoolcraft W B, Gardner D K. Vitrification of mouse and human blastocysts using a novel cryoloop container-less technique. Fertil Steril 1999; 72:1073-8.
36. Kuwayama M, Vajta G, Ieda S, Kato O. Comparison of open and closed methods for vitrification of human embryos and the elimination of potential contamination. Reprod Biomed Online 2005; 11:608-14.
37. Porcu E, Bazzocchi A, Notarangelo L, Paradisi R, Landolfo C, Venturoli S. Human oocyte cryopreservation in infertility and oncology. Curr Opin Endocrinol Diabetes Obes 2008; 15:529-35.
38. Borini A, Cattoli M, Bulletti C, Coticchio G. Clinical efficiency of oocyte and embryo cryopreservation. Ann N Y Acad Sci 2008; 1127:49-58.
39. Song Y S, Adler D, Xu F, Kayaalp E, Nureddin A, Anchan R M, Maas R L, Demirci U.
Vitrification and levitation of a liquid droplet on liquid nitrogen. Proc Natl Acad Sci USA 2010; 107:4596-600.
40. Demirci U, Montesano G. Cell encapsulating droplet vitrification. Lab Chip 2007; 7:1428-33.
41. Armitage W J, Rich S J. Vitrification of organized tissues. Cryobiology 1990; 27:483-91.
42. Fahy G M, MacFarlane D R, Angell C A, Meryman H T. Vitrification as an approach to cryopreservation. Cryobiology 1984; 21:407-26.
43. Fahy G M, Saur J, Williams R J. Physical problems with the vitrification of large biological systems. Cryobiology 1990; 27:492-510.
44. Kuleshova L L, Gouk S S, Hutmacher D W. Vitrification as a prospect for cryopreservation of tissue-engineered constructs. Biomaterials 2007; 28:1585-96.
45. Gao D Y, Liu J, Liu C, McGann L E, Watson P F, Kleinhans F W, Mazur P, Critser E S, Critser J K. Prevention of osmotic injury to human spermatozoa during addition and removal of glycerol. Hum Reprod 1995; 10:1109-22.
46. Gilmore J A, Liu J, Peter A T, Critser J K. Determination of plasma membrane characteristics of boar spermatozoa and their relevance to cryopreservation. Biol Reprod 1998; 58:28-36.
47. Liu J, Christian J A, Critser J K. Canine RBC osmotic tolerance and membrane permeability. Cryobiology 2002; 44:258-68.
48. Song Y S, Moon S, Hulli L, Hasan S K, Kayaalp E, Demirci U. Microfluidics for cryopreservation. Lab Chip 2009; 9:1874-81.
49. Elmoazzen H Y, Poovadan A, Law G K, Elliott J A, McGann L E, Jomha N M. Dimethyl sulfoxide toxicity kinetics in intact articular cartilage. Cell Tissue Bank 2007; 8:125-33.
50. Katkov, I I, Katkova N, Critser J K, Mazur P. Mouse spermatozoa in high concentrations of glycerol: chemical toxicity vs osmotic shock at normal and reduced oxygen concentrations. Cryobiology 1998; 37:325-38.
51. Wang X, Hua T C, Sun D W, Liu B, Yang G, Cao Y. Cryopreservation of tissue-engineered dermal replacement in Me2SO: Toxicity study and effects of concentration and cooling rates on cell viability. Cryobiology 2007; 55:60-5.
52. Fahy G M, Lilley T H, Linsdell H, Douglas M S, Meryman H T. Cryoprotectant toxicity and cryoprotectant toxicity reduction: in search of molecular mechanisms. Cryobiology 1990; 27:247-68.
53. He X M, Park E Y H, Fowler A, Yarmush M L, Toner M. Vitrification by ultra-fast cooling at a low concentration of cryoprotectants in a quartz micro-capillary: A study using murine embryonic stem cells. Cryobiology 2008; 56:223-32.
54. Demirci U. Using micro-electro-mechanical systems (MEMS) to develop diagnostic tools. J Vis Exp 2007:314.
55. Geckil H, Xu F, Zhang X, Moon S, Demirci U. Engineering hydrogels as extracellular matrix mimics. Nanomedicine (Lond) 2010; 5:469-84.
56. Zhang X, Catalano P N, Gurkan U A, Khimji I, Demirci U. Emerging Technologies in Medical Applications of Minimum Volume Vitrification. Nanomedicine (Lond) 2011; In press.
57. Seo M, Paquet C, Nie Z, Xu S, Kumacheva E. Microfluidic consecutive flow-focusing droplet generators. Soft Matter 2007; 3:7.
58. Moon S, Kim Y, Dong L, Lombardi M, Haeggstrom E, Jensen R V, Hsiao L, Demirci U. Drop-on-demand single cell isolation and total RNA analysis. PLoS ONE 2011; Accepted.
59. Demirci U, Montesano G. Single cell epitaxy by acoustic picolitre droplets. Lab Chip 2007; 7:1139-45.
60. Arav A, Yavin S, Zeron Y, Natan D, Dekel I, Gacitua H. New trends in gamete's cryopreservation. Mol Cell Endocrinol 2002; 187:77-81.
61. Samot J, Moon S, Lei Shao, Xiaohui Zhang, Feng Xu, YoungSeok Song, Hasan Onur Keles, Laura Matloff, Jordan Markel, Demirci U. Blood banking in living droplets. PLoS ONE 2011; Accepted.
62. Song Y S, Lin R L, Montesano G, Durmus N G, Lee G, Yoo S S, Kayaalp E, Haeggstrom E, Khademhosseini A, Demirci U. Engineered 3D tissue models for cell-laden microfluidic channels. Anal Bioanal Chem 2009; 395:185-93.
63. Moon S, Hasan S K, Song Y S, Xu F, Keles H O, Manzur F, Mikkilineni S, Hong J W, Nagatomi J, Haeggstrom E, Khademhosseini A, Demirci U. Layer by Layer 3d Tissue Epitaxy by Cell Laden Hydrogel Droplets. Tissue Eng Part C Methods 2009.
64. Boutron P, Mehl P. Theoretical prediction of devitrification tendency: determination of critical warming rates without using finite expansions. Cryobiology 1990; 27:359-77.
65. Jiao A, Han X, Critser J K, Ma H. Numerical investigation of transient heat transfer characteristics and vitrification tendencies in ultra-fast cell cooling process. Cryobiology 2006; 52:386-92.
66. Moon S, Kim Y, Dong L, Lombardi M, Haeggstrom E, Jensen R V, Hsiao L, Demirci U. Drop-on-demand single cell isolation and total RNA analysis. PLOS One 2011; 6.
67. Xu F, Sridharan B, Wang S Q, Gurkan U A, Syverud B, Demirci U. Embryonic Stem Cell Printing for Controllable Uniform Sized Embryoid Body Formation. Biomicrofluidics 2011; Accepted.
68. Cheng X, Irimia D, Dixon M, Ziperstein J C, Demirci U, Zamir L, Tompkins R G, Toner M, Rodriguez W R. A microchip approach for practical label-free CD4+ T-cell counting of HIV-infected subjects in resource-poor settings. J Acquir Immune Defic Syndr 2007; 45:257-61.
69. Zhang X, Khimji I, Gurkan U A, Safaee H, Catalano P, Keles H O, Kayaalp E, Demirci U. Lensless Imaging for Simultaneous Microfluidic Sperm Monitoring and Sorting. Lab on a Chip 2011.
70. Zhang X, Khimji I, Shao L, Safaee H, Desai K, Keles H O, Gurkan U A, Kayaalp E, Nureddin A, Anchan R M, Maas R L, Demirci U. Nanoliter Droplet Vitrification for Oocyte Cryopreservation. Nanomedicine 2011; Under review.
71. Arav A Y, S.; Zeron, J.; Natan D.; Dekel, I.; Gacitua H. New trends in gamete's cryopreservation. Mol Cell Endocrinol 2002; 187:77-81.
72. Eroglu A, J. R M, R. B, A. F, S. C, H. B, M. T. Intracellular trehalose improves the survival of cryopreserved mammalian cells Nature Biotechnology 2000; 18:163-7.
73. Demirci U, Montesano G. Cell Encapsulating Droplet Vitrification. Lab Chip 2007; 7:1428-33.
74. Kamel M. M S, Keles H. O., Manzur F., Lin R. L., Hasan S. K., Haeggstrom E., Kuritzkes D. R., Demirci U. Rapid Automated Cell Quantification on HIV Microfluidic Devices. Lab Chip 2009.
75. Song Y S, Adler D, Kayaalp E, Nureddin A, Anchan R M, Maas R L, Demirci U. Levitating Vitrification Droplets. Proceedings of the National Academy of Sciences 2009; Under review with minor revisions.
76. Benard A, Advani S G. Energy equation and the crystallization kinetics of semi-crystalline polymers: regimes of coupling. Int J Heat Mass Transfer 1995; 38:819-32.
77. Hogan B, Beddington R, Costantini F, Lacey E. Manipulating the Mouse Embryo: A Laboratory Manual. 2 ed: Cold Spring Harbor Laboratory Pr; 1995
78. Liu L, Nutter L M, Law N, McKerlie C. Sperm freezing and in vitro fertilization in three substrains of C57BL/6 mice. J Am Assoc Lab Anim Sci 2009; 48:39-43.
79. Moon S, Kim Y, Dong L, Lombardi M, Haeggstrom E, Jensen R V, Hsiao L, Demirci U. Drop-on-demand single cell isolation and total RNA analysis. PLOS One 2011; In press.
80. Uranga J A, Pedersen R A, Arechaga J. Parthenogenetic activation of mouse oocytes using calcium ionophores and protein kinase C stimulators. Int J Dev Biol 1996; 40:515-9.
81. Boiso I, Marti M, Santalo J, Ponsa M, Barri P N, Veiga A. A confocal microscopy analysis of the spindle and chromosome configurations of human oocytes cryopreserved at the germinal vesicle and metaphase II stage. Hum Reprod 2002; 17:1885-91.
82. Lane M, Gardner D K. Vitrification of mouse oocytes using a nylon loop. Mol Reprod Dev 2001; 58:342-7.
83. Wang Z, Sun Z, Chen Y, He F. A modified cryoloop vitrification protocol in the cryopreservation of mature mouse oocytes. Zygote 2009; 17:217-24.
84. Griffin J, Emery B R, Huang I, Peterson C M, Carrell D T. Comparative analysis of follicle morphology and oocyte diameter in four mammalian species (mouse, hamster, pig, and human). Exp Clin Assist Reprod 2006; 3:2.
85. McBurnie L D, Bardo B. Validation of sterile filtration of liquid nitrogen. Pharm Technol North America 2002; 26:9.
86. Parmegiani L, Cognigni G E, Filicori M. Ultra-violet sterilization of liquid nitrogen prior to vitrification. Hum Reprod 2009; 24:2969.
87. Bielanski A, Vajta G. Risk of contamination of germplasm during cryopreservation and cryobanking in IVF units. Hum Reprod 2009; 24:2457-67.

### Example 5

### Discussion

Described herein are systems and methods relating to ejector-based droplet-vitrification for cryopreservation of cells. The systems and methods have increased survivability and function compared to slow freezing and conventional straw-based vitrification methods. Further, prior droplet-vitrification methods have limited control over the droplet size, preventing a uniform size distribution among the generated droplets, which causes in inconsistencies in droplet vitrification. Described herein are systems and methods relating to ejector-based droplet-vitrification, with which a single to few cells could be encapsulated in reduced droplet volumes (1.5-500 nL). This droplet generation and ejection technology has been demonstrated to generate droplets at high-throughput with rates up to 1000 droplets per second. With this method, droplets maintain their spherical shape when ejected into liquid nitrogen. This may provide even cooling of droplets from surface to center compared to other droplet-vitrification approaches, such as solid surface vitrification, in which hemispheres were generated instead of spheres.

TABLE 1
Composition of the cryoprotective solutions used (g/40 ml).

| CPA | Glycerol (ml) | Sorbitol (g) | NaCl (g) | Sterile DI Water (ml) |
|---|---|---|---|---|
| 1.0M | 2.9 | 1.16 | 0.25 | 37.1 |
| 2.0M | 5.8 | 1.16 | 0.25 | 34.2 |
| 2.5M | 7.3 | 1.16 | 0.25 | 32.7 |
| 4.0M | 11.7 | 1.16 | 0.25 | 28.3 |

TABLE 2
Spectrometer absorbance values for the two controls and actual sample from each step in the cryopreservation process
from experimental conditions of 75 mm droplet collecting distance and 4.0 l/min of sheath gas flow rate).

| | | Processes | | | | | |
|---|---|---|---|---|---|---|---|
| | | CPA loading | | Cryopreservation | | | |
| Absorbance | | CPA₁ | CPA₂ | Ejection | Collection Film | Freezing | Total % Hemolysis |
| λ_{416 mm} | ABS₀ | 0.008 ± 0.001 | 0.051 ± 0.047 | 0.027 ± 0.021 | 0.700 ± 0.043 | 0.407 ± 0.045 | |
| | ABS₁₀₀ | 3.849 ± 0.158 | 4.050 ± 0.187 | 1.833 ± 0.013 | 1.798 ± 0.061 | 1.124 ± 0.081 | |
| | ABS | 0.051 ± 0.047 | 0.122 ± 0.029 | 0.229 ± 0.020 | 0.631 ± 0.052 | 0.462 ± 0.053 | |
| λ_{545 mm} | ABS₀ | −0.006 ± 0.003 | 0.006 ± 0.014 | −0.007 ± 0.017 | 0.098 ± 0.023 | 0.046 ± 0.012 | |
| | ABS₁₀₀ | 0.447 ± 0.026 | 0.477 ± 0.019 | 0.205 ± 0.002 | 0.193 ± 0.007 | 0.119 ± 0.009 | |
| | ABS | 0.006 ± 0.014 | 0.037 ± 0.019 | 0.000 ± 0.011 | 0.079 ± 0.013 | 0.051 ± 0.013 | |
| λ_{576 mm} | ABS₀ | −0.006 ± 0.002 | 0.006 ± 0.014 | −0.006 ± 0.016 | 0.103 ± 0.023 | 0.049 ± 0.012 | |
| | ABS₁₀₀ | 0.486 ± 0.028 | 0.524 ± 0.021 | 0.223 ± 0.002 | 0.214 ± 0.008 | 0.132 ± 0.010 | |
| | ABS | 0.006 ± 0.014 | 0.037 ± 0.019 | 0.004 ± 0.011 | 0.084 ± 0.012 | 0.055 ± 0.012 | |
| Cripps | ABS₀ | 0.001 ± 0.000 | 0.004 ± 0.002 | 0.003 ± 0.001 | 0.047 ± 0.003 | 0.026 ± 0.003 | |
| | ABS₁₀₀ | 0.291 ± 0.018 | 0.320 ± 0.015 | 0.134 ± 0.002 | 0.135 ± 0.005 | 0.085 ± 0.006 | |
| | ABS | 0.004 ± 0.002 | 0.007 ± 0.001 | 0.019 ± 0.001 | 0.041 ± 0.002 | 0.032 ± 0.002 | |
| Harboe | ABS₀ | 0.014 ± 0.003 | 0.042 ± 0.029 | 0.035 ± 0.008 | 0.552 ± 0.018 | 0.332 ± 0.031 | |
| | ABS₁₀₀ | 3.167 ± 0.122 | 3.311 ± 0.159 | 1.517 ± 0.011 | 1.491 ± 0.050 | 0.931 ± 0.067 | |
| | ABS | 0.042 ± 0.029 | 0.079 ± 0.015 | 0.214 ± 0.005 | 0.532 ± 0.052 | 0.381 ± 0.038 | |
| % Hemolysis | λ_{416 mm} | 29.23 ± 0.91% | 1.79 ± 0.80% | 11.18 ± 0.39% | −6.29 ± 6.10% | 7.58 ± 2.51% | 43.49 ± 10.71% |
| | λ_{545 mm} | 2.65 ± 3.05% | 6.57 ± 4.37% | 3.61 ± 2.37% | −19.93 ± 23.36% | 5.94 ± 16.56% | −1.16 ± 52.71% |
| | λ_{576 mm} | 2.44 ± 2.80% | 5.92 ± 4.06% | 4.22 ± 2.06% | −17.07 ± 22.01% | 7.20 ± 12.65% | 2.71 ± 43.58% |
| | Cripps | 0.92 ± 0.49% | 0.95 ± 0.11% | 11.83 ± 0.82% | −6.82 ± 4.68% | 8.88 ± 1.52% | 15.76 ± 7.61% |
| | Harboe | 0.90 ± 0.88% | 1.13 ± 0.42% | 12.10 ± 0.46% | −2.09 ± 6.90% | 8.31 ± 1.13% | 20.35 ± 9.80% |

TABLE 3
Percent hemolysis values of ejection, collection film, and
freezing for five different experimental conditions are given.
Total hemolysis is the sum of hemolysis due to the ejection
and freezing steps:

| | | | | | |
|---|---|---|---|---|---|
| Flow rate | | | Collection | | |
| (l/min) | Methods | Ejection | Film | Freezing | Total |

Distance (mm) 60

| | | | | | |
|---|---|---|---|---|---|
| 3.2 | λ_{1, 416 nm} | 7.92% | −7.01% | 8.27% | |
| | λ_{2, 545 nm} | 9.26% | −5.91% | 48.31% | |
| | λ_{3, 576 nm} | 8.88% | −5.77% | 44.74% | |
| | Cripps | 6.87% | −6.38% | 5.20% | 14.07% |
| | Harboe | 7.46% | −1.63% | 3.85% | 11.31% |
| 4.0 | | | | | |
| 4.8 | λ_{1, 416 nm} | 17.48% | −2.02% | 1.76% | |
| | λ_{2, 545 nm} | 19.15% | −3.75% | 5.13% | |
| | λ_{3, 576 nm} | 18.92% | −4.09% | 4.55% | |
| | Cripps | 16.16% | −4.07% | 1.60% | 17.76% |
| | Harboe | 16.97% | −2.02% | 1.22% | 18.19% |

Distance (mm) 75

| | | | | | |
|---|---|---|---|---|---|
| 3.2 | | | | | |
| 4.0 | λ_{1, 416 nm} | 11.18% | −6.29% | 7.58% | |
| | λ_{2, 545 nm} | 3.61% | −19.93% | 5.94% | |
| | λ_{3, 576 nm} | 4.22% | −17.07% | 7.20% | |
| | Cripps | 11.83% | −6.82% | 8.88% | 20.71% |
| | Harboe | 12.10% | −2.09% | 8.31% | 20.51% |
| 4.8 | | | | | |

Distance (mm) 90

| | | | | | |
|---|---|---|---|---|---|
| 3.2 | λ_{1, 416 nm} | 9.43% | −9.82% | 5.85% | |
| | λ_{2, 545 nm} | 12.09% | −12.71% | 15.06% | |
| | λ_{3, 576 nm} | 11.94% | −12.39% | 13.96% | |
| | Cripps | 8.78% | −9.28% | 5.47% | 14.25% |
| | Harboe | 9.00% | −4.16% | 4.14% | 13.14% |
| 4.0 | | | | | |
| 4.8 | λ_{1, 416 nm} | 15.97% | −5.36% | 6.05% | |
| | λ_{2, 545 nm} | 13.81% | −4.83% | 13.60% | |
| | λ_{3, 576 nm} | 13.72% | −4.96% | 11.98% | |
| | Cripps | 14.74% | −6.74% | 3.02% | 17.76% |
| | Harboe | 16.36% | −2.31% | 4.02% | 20.38% |

TABLE 4
Cryopreservation process for multiple ejectors (4 ejectors).

| | | Cryopreservation | | |
|---|---|---|---|---|
| Absorbance | | Ejection | Freezing | Total % Hemolysis |
| λ_{416 mm} | ABS₀ | 0.008 ± 0.001 | 0.373 ± 0.019 | |
| | ABS₁₀₀ | 3.849 ± 0.158 | 1.861 ± 0.073 | |
| | ABS | 0.710 ± 0.051 | 0.416 ± 0.021 | |
| λ_{545 mm} | ABS₀ | −0.006 ± 0.003 | 0.035 ± 0.007 | |
| | ABS₁₀₀ | 0.447 ± 0.026 | 0.196 ± 0.011 | |
| | ABS | 0.105 ± 0.035 | 0.041 ± 0.011 | |
| λ_{576 mm} | ABS₀ | −0.006 ± 0.002 | 0.038 ± 0.008 | |
| | ABS₁₀₀ | 0.486 ± 0.028 | 0.217 ± 0.011 | |
| | ABS | 0.111 ± 0.034 | 0.043 ± 0.011 | |
| Cripps | ABS₀ | 0.001 ± 0.000 | 0.023 ± 0.002 | |
| | ABS₁₀₀ | 0.291 ± 0.018 | 0.138 ± 0.005 | |
| | ABS | 0.048 ± 0.002 | 0.025 ± 0.002 | |
| Harboe | ABS₀ | 0.014 ± 0.003 | 0.315 ± 0.016 | |
| | ABS₁₀₀ | 3.167 ± 0.122 | 1.542 ± 0.056 | |
| | ABS | 0.561 ± 0.026 | 0.344 ± 0.009 | |
| % Hemolysis | λ_{416 mm} | 18.28% ± 2.03% | 2.85% ± 2.63% | 21.13% ± 4.66% |
| | λ_{545 mm} | 24.54% ± 8.64% | 3.73% ± 8.83% | 28.28% ± 17.47% |
| | λ_{576 mm} | 23.70% ± 7.68% | 2.79% ± 8.62% | 26.49% ± 16.30% |
| | Cripps | 16.12% ± 1.66% | 1.45% ± 1.90% | 17.58% ± 3.56% |
| | Harboe | 17.35% ± 1.47% | 2.40% ± 1.82% | 19.76% ± 3.29% |

TABLE 5
Cryopreservation process for multiple ejectors (25 ejectors).

| | | Cryopreservation | | |
|---|---|---|---|---|
| Absorbance | | Ejection | Freezing | Total % Hemolysis |
| λ_{416 mm} | ABS₀ | 0.066 ± 0.005 | 0.534 ± 0.043 | |
| | ABS₁₀₀ | 1.562 ± 0.027 | 4.050 ± 0.187 | |
| | ABS | 0.334 ± 0.030 | 0.582 ± 0.016 | |
| λ_{545 mm} | ABS₀ | 0.014 ± 0.002 | 0.057 ± 0.004 | |
| | ABS₁₀₀ | 0.164 ± 0.003 | 0.477 ± 0.019 | |
| | ABS | 0.051 ± 0.038 | 0.068 ± 0.011 | |
| λ_{576 mm} | ABS₀ | 0.014 ± 0.002 | 0.063 ± 0.005 | |
| | ABS₁₀₀ | 0.181 ± 0.003 | 0.524 ± 0.021 | |
| | ABS | 0.053 ± 0.036 | 0.075 ± 0.010 | |
| Cripps | ABS₀ | 0.004 ± 0.001 | 0.038 ± 0.004 | |
| | ABS₁₀₀ | 0.117 ± 0.001 | 0.320 ± 0.015 | |
| | ABS | 0.022 ± 0.001 | 0.043 ± 0.003 | |
| Harboe | ABS₀ | 0.048 ± 0.005 | 0.446 ± 0.036 | |
| | ABS₁₀₀ | 1.305 ± 0.024 | 3.311 ± 0.159 | |
| | ABS | 0.259 ± 0.014 | 0.474 ± 0.024 | |
| % Hemolysis | λ_{416 mm} | 17.91% ± 1.99% | 1.36% ± 1.27% | 19.27% ± 3.26% |
| | λ_{545 mm} | 24.67% ± 24.43% | 2.70% ± 3.62% | 27.36% ± 28.05% |
| | λ_{576 mm} | 23.51% ± 20.91% | 2.67% ± 3.12% | 26.18% ± 24.04% |
| | Cripps | 16.25% ± 1.39% | 1.83% ± 1.20% | 18.08% ± 2.59% |
| | Harboe | 16.80% ± 1.15% | 0.97% ± 1.18% | 17.77% ± 2.33% |

TABLE 6
Nonparametric Mann-Whitney U test results (p-values) of pairwise comparisons for
ejection at two different distances (60 and 90 mm) and gas flow rates (3.2 and
4.8 l/min) for Cripps method(*) and Harboe method(**). Freezing was not affected
from the ejection conditions as per nonparametric Kruskal-Wallis one way analysis
of variance, therefore pairwise comparisons were not performed.

| Mann-Whitney U test | 60/3.2-60/4.8 | 90/3.2-90/4.8 | 60/3.2-90/3.2 | 60/4.8-90/4.8 |
|---|---|---|---|---|
| Cripps method(*) | P₁ | P₂ | P₃ | P₄ |
| Ejection | 0.04 | 0.04 | 0.26 | 0.33 |
| Freezing | — | — | — | — |
| Harboe method(**) | P₁ | P₂ | P₃ | P₄ |
| Ejection | 0.04 | 0.04 | 0.26 | 0.33 |
| Freezing | — | — | — | — |

TABLE 7
Appendix for symbols

| | |
|---|---|
| WB | Whole blood |
| RBC | Red blood cell |
| CPA | Cryoprotective agent |
| CPDA-1 | Citrate phosphate dextrose adenine |
| DPBS | Dulbecco's Phosphate Buffered Saline |
| CPA₁ | Mixture of RBCs and final 1M glycerol concentration after first CPA loading step |
| CPA₂ | Mixture of RBCs and final 2.5M glycerol concentration after second CPA loading step |
| ABS₀ | Absorbance of free hemoglobin before each process |
| ABS₁₀₀ | Absorbance of free hemoglobin with 100% hemolysis |
| ABS_{process} | Absorbance of free hemoglobin after each process |
| ABS₀_{_{—}}_{CPA}_{₁} | Absorbance of free hemoglobin before loading CPA1 |
| ABS₁₀₀_{_{—}}_{CPA}_{₁} | Absorbance of free hemoglobin before loading CPA1 with 100% hemolysis |
| ABS_{CPA}_{₁} | Absorbance of free hemoglobin after loading CPA1 |
| ABS₀_{_{—}}_{CPA}_{₂} | Absorbance of free hemoglobin before loading CPA2 |
| ABS₁₀₀_{_{—}}_{CPA}_{₂} | Absorbance of free hemoglobin before loading CPA2 with 100% hemolysis |
| ABS_{CPA}_{₂} | Absorbance of free hemoglobin after loading CPA2 |
| ABS₀_{_{—}}_{ejection} | Absorbance of free hemoglobin before ejection |
| ABS₁₀₀_{_{—}}_{ejection} | Absorbance of free hemoglobin before ejection with 100% hemolysis |
| ABS_{ejection} | Absorbance of free hemoglobin after ejection |
| ABS_{0 film} | Absorbance of free hemoglobin ejected into 2.5M glycerol solution |
| ABS_{100 film} | Absorbance of free hemoglobin ejected into DI water |
| ABS_{film} | Absorbance of free hemoglobin ejected onto collection film |
| ABS_{0 fs} | Absorbance of free hemoglobin ejected onto collection film water and then dipping into 2.5M |
| | glycerol |
| ABS_{100 fs} | Absorbance of free hemoglobin ejected onto collection film and then dipping into DI |
| ABS_{fs} | Absorbance of free hemoglobin ejected onto PE film and then freeze and thaw into 2.5M |
| | glycerol |
| ABS₀_{_{—}}_{ejector}_{_{—}}ₓ₂₅ | Absorbance of free hemoglobin before ejection using 25 ejector system |
| ABS₁₀₀_{_{—}}_{ejector}_{_{—}}ₓ₂₅ | Absorbance of free hemoglobin before ejection with 100% hemolysis using 25 ejector system |
| ABS_{ejector}_{_{—}}ₓ₂₅ | Absorbance of free hemoglobin after ejection using 25 ejector system |
| ABS_{0 film x25} | Absorbance of free hemoglobin ejected into 2.5M glycerol solution using 25 ejector system |
| ABS_{100 film x25} | Absorbance of free hemoglobin ejected into DI water using 25 ejector system |
| ABS_{film x25} | Absorbance of free hemoglobin ejected onto collection film using 25 ejector system |
| ABS_{0 fs x25} | Absorbance of free hemoglobin ejected onto collection film water and then immersing into 2.5M |
| | glycerol using 25 ejector system |
| ABS_{200 f3 x25} | Absorbance of free hemoglobin ejected onto collection film and then immersing into DI using 25 |
| | ejector system |

TABLE 8
Oocyte survival after CPA loading/unloading and
ejection with CPA.

| | | No. of | |
|---|---|---|---|
| | No. of | oocytes | Survival |
| | oocytes | survived | rate after |
| | initiated | after 24 h | 24 h (%) |
| Control (Fresh) | 99 | 94 | 94.9 |
| CPA Loading/Unloading | 102 | 94 | 92.2 |
| Ejection with CPA | 99 | 89 | 89.9 |

TABLE 9
Parthenogenetic activation using SrCl₂ solution for oocytes
retrieved after harvesting (control) and after encapsulation in
medium droplets followed by ejection into medium.

| | SrCl₂ | No. of | No. of | Blastocysts |
|---|---|---|---|---|
| Experimental | Concentration | Oocytes | Oocytes | developed |
| Groups | (mM) | initiated | cleaved (%) | (%) |
| Control | 10 | 81 | 3 (3.7%) | 0 (0) |
| | 20 | 82 | 38 (46.3%) | 13 (15.9%) |
| | 50 | 79 | 70 (88.6%)^{a} | 33 (49.4%)^{b} |
| | 100 | 88 | 43 (48.9%) | 2 (2.3%) |
| Ejection with | 50 | 100 | 96 (97.0%)^{a} | 26 (26.0%)^{b} |
| medium | | | | |

^{a,b}values with the same superscript letters are significantly different (p < 0.01).

What is claimed herein is:

1. A method of vitrifying a biological sample comprising;
generating nanodroplets of a solution comprising the biological sample; and contacting the nanodroplets with a cooling agent.

2. The method of claim 1, wherein the biological sample is selected from the group consisting of:
cells; biological fluids; biopsy samples; diagnostic samples; blood; urine; and protein.

3. The method of claim 2, wherein the cells are selected from the group consisting of:
gametes; sperm; eggs; embryos; zygotes; chondrocytes; red blood cells; blood cells, hepatic cells, fibroblasts, stem cells; cord blood cells; adult stem cells, induced pluripotent stem cells, autologous cells; autologous stem cells; bone marrow cells; hematopoietic cells; embryonic stem cells; and hematopoietic stem cells.

4. The method of claim 1, wherein the nanodroplets have a volume of less than 500 nL.

5. The method of claim 1, wherein the nanodroplets have a volume of less than 100 nL.

6. The method of claim 1, wherein the nanodroplets have a volume of less than 10 nL.

7. The method of claim 1, wherein the solution comprising the biological sample further comprises at least one cryoprotective agent.

8. The method of claim 7, wherein the cryoprotective agent is selected from the group consisting of:
dimethylsulphoxide (DMSO), 1,2-propanediol (PROH), ethylene glycol (EG), sucrose, trehalose; mannitol; ectoin; methylcellulose; polyethylene glycol (PEG); and naturally occurring cyroprotectants.

9. The method of claim 7, wherein the cryoprotective agent is present at a concentration of less than 6 M.

10. The method of claim 7, wherein the cryoprotective agent is present at a concentration of less than 3 M.

11. The method of claim 7, wherein the cryoprotective agent is present at a concentration of less than 2 M.

12. The method of claim 1, wherein the solution comprising the biological sample further comprises a hydrogel.

13. The method of claim 1, wherein the nanodroplets are generated by causing the solution comprising the biological sample to flow through a nozzle of a reservoir.

14. The method of claim 13, wherein the solution comprising the biological sample is caused to flow through the nozzle of the reservoir via a means selected from the group consisting of:
a plunger; a solenoid-controlled plunger; co-flow of a gas; an inkjet, and spraying.

15. The method of claim 1, wherein the means of generating the nanodroplets is an acoustic generator.

16. The method of claim 1, wherein the means of generating nanodroplets is automated.

17. The method of claim 1, wherein the nanodroplets are contacted with the cooling agent by allowing the nanodroplets to fall from the nozzle into or onto a cooling agent.

18. The method of claim 1, wherein the nanodroplets are contacted with the cooling agent by allowing the nanodroplets to fall from the nozzle onto a collection membrane and then contacting the collection membrane with the cooling agent.

19. The method of claim 1, wherein the cooling agent is selected from the group consisting of:
liquid nitrogen, nitrogen vapor, liquid helium, and helium vapor.

20. The method of claim 1, further comprising storing the vitrified biological sample at a temperature lower than −130° C.

21. The method of claim 1, further comprising generating the nanodroplets in a high throughput system.

22. The method of claim 21, wherein the high throughput system comprises a reservoir with multiple nozzles.

23. The method of claim 21, wherein the high throughput system comprises multiple reservoirs.

24. The method of claim 1, further comprising causing the vitrified biological sample to warm rapidly.

25. A system for vitrifying a biological sample comprising;
a reservoir containing the biological sample;
a means of forming the biological sample into nanodroplets and directing the nanodroplets to flow or fall towards a catchment;
a catchment for collecting the nanodroplets.

26. The system of claim 25, wherein the means of forming the biological sample into nanodroplets comprises causing a solution comprising the biological sample to flow through a nozzle.

27. The system of claim 26, wherein a means of causing the solution comprising the biological sample to flow through the nozzle connected to the reservoir is selected from a group consisting of:
a plunger; a solenoid-controlled plunger; a gas co-flow muzzle; and spraying.

28. The system of claim 25, wherein the means of forming the biological sample into the nanodroplets is an acoustic generator.

29. The system of claim 25, wherein the means of forming the biological sample into the nanodroplets is automated.

30. The system of claim 26, wherein a terminus of the nozzle is less than 200 μm in diameter.

31. The system of claim 25, wherein the catchment comprises a cooling agent.

32. The system of claim 31, wherein the cooling agent is selected from the group consisting of:
liquid nitrogen, nitrogen vapor, liquid helium, and helium vapor.

33. The system of claim 25, wherein the catchment comprises a collection membrane.

34. The system of claim 25, wherein the nanodroplets have a volume of less than 500 nL.

35. The system of claim 25, wherein the nanodroplets have a volume of less than 100 nL.

36. The system of claim 25, wherein the nanodroplets have a volume of less than 10 nL.

37. The system of claim 25, wherein the biological sample further comprises at least one cryoprotective agent.

38. The system of claim 37, wherein the cryoprotective agent is selected from the group consisting of:
dimethylsulphoxide (DMSO), 1,2-propanediol (PROH), ethylene glycol (EG), sucrose, trehalose; mannitol; ectoin; methylcellulose; polyethylene glycol (PEG); and naturally occurring cyroprotectants.

39. The system of claim 37, wherein the cryoprotective agent is present at a concentration of less than 6 M.

40. The system of claim 37, wherein the cryoprotective agent is present at a concentration of less than 3 M.

41. The system of claim 37, wherein the cryoprotective agent is present at a concentration of less than 2 M.

42. The system of claim 25, wherein the biological sample further comprises a hydrogel.

43. The system of claim 25, wherein the system is a high throughput system.

44. The system of claim 43, wherein the reservoir of the high throughput system comprises multiple nozzles.

45. The system of claim 44, wherein the high throughput system comprises multiple reservoirs.
